(19) United States
(12) Patent Application Publication
(10) Pub. No.: US 2023/0036065 A1
(43) Pub. Date: Feb. 2, 2023

(54) METHODS FOR CANCER IMMUNOTHERAPY

(71) Applicant: Precision BioSciences, Inc., Durham, NC (US)

(72) Inventors: Bruce J. McCreedy, Jr., Raleigh, NC (US); Derek Jantz, Durham, NC (US); Aaron Martin, Carrboro, NC (US); Daniel T. MacLeod, La Jolla, CA (US)

(73) Assignee: Precision BioSciences, Inc., Durham, NC (US)

(21) Appl. No.: 17/782,607

(22) PCT Filed: Dec. 3, 2020
(86) PCT No.: PCT/US2020/063159

§ 371 Date: Jun. 3, 2022

Related U.S. Application Data
(60) Provisional application No. 62/987,752, filed on Mar. 10, 2020.
(60) Provisional application No. 62/961,629, filed on Jan. 15, 2020.
(60) Provisional application No. 62/945,811, filed on Dec. 9, 2019.
(60) Provisional application No. 62/944,868, filed on Dec. 6, 2019.

(51) Int. Cl. A61K 35/17; A61K 39/395; A61K 31/664; A61K 31/7076; C07K 16/28; A61K 38/17; A61P 35/00; A61P 35/02

(52) U.S. Cl. CPC A61K 35/17; A61K 39/3955; A61K 31/664; A61K 31/7076; C07K 16/2803; C07K 16/2887; C07K 16/2878; A61K 38/1774; A61P 35/00; A61P 35/02; C07K 2317/622; A61K 2039/505

(57) ABSTRACT

The present invention encompasses methods of cancer immunotherapy, and particularly methods of allogeneic cellular immunotherapy, using particular lymphodepletion regimens in combination with particular populations of chimeric antigen receptor T cells expressing anti CD19 CAR PBCAR0191, anti CD20 CAR PBCAR20A or anti BCMA CAR PBCAR269A.

### FIELD OF THE INVENTION

The invention relates to the field of oncology and cancer immunotherapy. In particular, the invention relates to allogeneic cellular immunotherapy and lymphodepletion regimens.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 3, 2020, is named P109070047WO00-SEQ-EPG, and is 29 kilobytes in size.

### BACKGROUND OF THE INVENTION

T cell adoptive immunotherapy is a promising approach for cancer treatment. The immunotherapy treatment methods disclosed herein utilize isolated human T cells that have been genetically-modified to enhance their specificity for a specific tumor associated antigen. Genetic modification may involve the expression of a chimeric antigen receptor or an exogenous T cell receptor to graft antigen specificity onto the T cell. In contrast to exogenous T cell receptors, chimeric antigen receptors derive their specificity from the variable domains of a monoclonal antibody. Thus, T cells expressing chimeric antigen receptors (CAR T cells) induce tumor immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Despite its potential usefulness as a cancer treatment, adoptive immunotherapy with CAR T cells has been limited, in part, by expression of the endogenous T cell receptor on the cell surface. CAR T cells expressing an endogenous T cell receptor may recognize major and minor histocompatibility antigens following administration to an allogeneic patient, which can lead to the development of graft-versus-host-disease (GVHD). As a result, clinical trials have largely focused on the use of autologous CAR T cells, wherein a patient's T cells are isolated, genetically-modified to incorporate a chimeric antigen receptor, and then re-infused into the same patient. An autologous approach provides immune tolerance to the administered CAR T cells; however, this approach is constrained by both the time and expense necessary to produce patient-specific CAR T cells after a patient's cancer has been diagnosed.

Thus, it would be advantageous to develop “off the shelf” CAR T cells, prepared using T cells from a third party, healthy donor, that have reduced expression, or have no detectable cell surface expression of an endogenous T cell receptor (e.g., an alpha/beta T cell receptor) and do not initiate GvHD upon administration. Such products could be generated and validated in advance of diagnosis and could be made available to patients as soon as necessary. Therefore, a need exists for the development of allogeneic CAR T cells that lack an endogenous T cell receptor in order to prevent the occurrence of GvHD.

Currently, prior to CAR T cell therapy, patients are pre-treated with a round of chemotherapy for purposes of lymphodepletion. The chemotherapeutic lymphodepletion agents typically are fludarabine, cyclophosphamide, or a combination thereof. This is typically carried out 3 days to 1 week prior to injection with the CAR T cells. In terms of autologous cell therapy this is generally sufficient to eliminate enough of the host lymphocytes to make space for the incoming CAR T cells to benefit from the microenvironment of the host and promote expansion of the incoming CAR T cells (see Hay et al., Drugs (2017) 77(3): 237-245).

Treatment is more complicated with allogeneic CAR T cells because of the higher potential for host vs. graft rejection of the injected CAR T cells. Insufficient lymphodepletion can cause the host to elicit an immune response against the CAR T cells and limit their ability to expand and limit efficacy. One approach to overcoming this problem is to utilize a biological lymphodepletion agent, such as a monoclonal antibody, or other agent that targets host immune cells but does not target the CAR T cell. Poirot et al., describes an approach where CAR T cells were engineered using TALENs to generate cells deficient in both the αβ T cell receptor and a second protein CD52, which is expressed on host lymphocytes (see Poirot et al., Cancer Research (2015) 18(75)). The authors then utilized an anti-CD52 antibody to further deplete host lymphocytes.

Results from clinical trials using TCR/CD52 double-knockout CAR T cells have suggested that it is necessary to include a biological lymphodepletion agent, such as the anti-CD52 antibody alemtuzumab, as part of the lymphodepletion regimen in order to achieve clinical responses. For example, pooled data from phase 1 studies in pediatric and adult patients having relapsed/refractory B-cell acute lymphoblastic leukemia (ALL) were reported in 2018 and 2019 (Benjamin et al., Blood (2018) 132 (Supplement 1): 896, doi.org/10.1182/blood-2018-99-111356; Benjamin R., Clinical Advances in Hematology and Oncology (2019) 17(3), 155-157). In these studies, patients were administered TCR/CD52 double KO CAR T cells following a lymphodepletion regimen (administered one week prior to CAR T infusion) that included fludarabine (90 mg/m² in adult patients, 150 mg/m² in pediatric patients) and cyclophosphamide (1500 mg/m² in adult patients, 90 mg/m² in pediatric patients) with or without the anti-CD52 antibody alemtuzumab (1 mg/kg). Notably, in all patients who received fludarabine and cyclophosphamide only, and no alemtuzumab, there were no reported responses and no CAR T expansion. By contrast, a complete remission, or complete remission with incomplete blood recovery was observed in 14 of 17 patients (82%) whose lymphodepletion regimen included alemtuzumab. These results suggested that a biological lymphodepletion agent was necessary for responses in allogeneic CAR T therapy, and for CAR T expansion following administration.

However, there are drawbacks to the inclusion of biological lymphodepletion agents in the lymphodepletion regimen. For example, CD52 antibodies, such as alemtuzumab, can have a long half-life and can be associated with toxicities, cytopenias, and infections. Therefore, a need currently exists for allogeneic CAR T therapies that provide a simpler lymphodepletion regimen that requires the use of minimal amounts of these biological lymphodepletion agents.

### SUMMARY OF THE INVENTION

In one aspect, the invention provides a method of immunotherapy for treating cancer in a subject, the method comprising: (a) administering to the subject a lymphodepletion regimen that includes no greater than a minimal effective dose of any biological lymphodepletion agent, wherein the lymphodepletion regimen comprises one or more chemotherapeutic lymphodepletion agents; and (b) administering to the subject an effective dose of a pharmaceutical composition comprising a population of human T cells, wherein a plurality of the human T cells are chimeric antigen receptor (CAR) T cells expressing a cell surface CAR, wherein a T cell receptor (TCR) alpha gene or a TCR beta gene is inactivated in the CAR T cells, and wherein the pharmaceutical composition is administered at a dose of between about 3×10⁴ to about 1×10⁷ CAR T cells/kg; wherein the lymphodepletion regimen is administered prior to administration of the pharmaceutical composition, wherein the CAR comprises an extracellular ligand-binding domain having specificity for a cancer cell antigen, a hinge domain, a transmembrane domain, a co-stimulatory domain, and an intracellular signaling domain, and wherein the population of human T cells comprises one or more of the following characteristics: (i) the CAR T cells represent between more than about 40% of cells in the population of human T cells; (ii) the ratio of CD4+ CAR T cells to CD8+ CAR T cells in the population is greater than about 0.5; (iii) the percentage of CD4+ CAR T cells in the population that are also CCR7+ is greater than about 35% and (iv) the percentage of CD8+ CAR T cells in the population that are also CCR7+ is greater than about 25%. In such an aspect, the population of human T cells can comprise any combination of the recited characteristics including: (i) alone; (ii) alone; (iii) alone; (iv) alone; a combination of (i) and (ii); a combination of (i) and (iii); a combination of (i) and (iv); a combination of (ii) and (iii); a combination of (ii) and (iv); a combination of (iii) and (iv); a combination of (i), (ii), and (iii); a combination of (i), (ii), and (iv); a combination of (i), (iii), and (iv); or a combination of (ii), (iii), and (iv). In a particular embodiment, the method comprises characteristics (i), (ii), (iii), and (iv).

In some embodiments, (i) the CAR T cells represent between about 40% and 75% of cells in the population of human T cells; (ii) the ratio of CD4+ CAR T cells to CD8+ CAR T cells in the population is between about 0.5 and about 3.0; (iii) the percentage of CD4+ CAR T cells in the population that are also CCR7+ is between about 35% to about 75%; and/or (iv) the percentage of CD8+ CAR T cells in the population that are also CCR7+ is between about 25% and 75%.

In some embodiments, the pharmaceutical composition comprising a population of human T cells is referred to as PBCAR0191, which comprises a plurality of CAR T cells expressing a CD19-specific CAR. In some embodiments, the pharmaceutical composition comprising a population of human T cells is referred to as PBCAR20A, which comprises a plurality of CAR T cells expressing a CD20-specific CAR. In some embodiments, the pharmaceutical composition comprising a population of human T cells is referred to as PBCAR269A, which comprises a plurality of CAR T cells expressing a BCMA-specific CAR.

In some embodiments, the method further comprises administering a second dose of the pharmaceutical composition to the subject. In some embodiments, the method comprises administering a second dose of the pharmaceutical composition without re-administration of the lymphodepletion regimen. In some such embodiments, the method comprises administering a second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition. In certain embodiments, the method comprises administering a second dose of the pharmaceutical composition 10 days following administration of the first dose of the pharmaceutical composition. In some embodiments, the second dose of the pharmaceutical composition is administered at the same dose of CAR T cells/kg as administered in the first dose of the pharmaceutical composition. In some embodiments, the second dose of the pharmaceutical composition is administered at a different dose of CAR T cells/kg than administered in the first dose of the pharmaceutical composition.

In some embodiments, the method comprises administering a second dose of the pharmaceutical composition and a third dose of the pharmaceutical composition. In some embodiments, the method comprises administering a second dose of the pharmaceutical composition and a third dose of the pharmaceutical composition without re-administration of the lymphodepletion regimen. In some such embodiments, the method comprises administering a second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition, and further comprises administering a third dose of the pharmaceutical composition 1, 2, 3, 4, 5, 6, 7, 8, 9, 10, 11, 12, 13, 14, 15, 16, 17, 18, 19, 20, or 21 days following administration of the second dose of the pharmaceutical composition. In some embodiments, the second dose of the pharmaceutical composition is administered 10 days following administration of the first dose of the pharmaceutical composition, and the third dose of the pharmaceutical composition is administered 4 days following administration of the second dose of the pharmaceutical composition. In some embodiments, the second dose of the pharmaceutical composition and the third dose of the pharmaceutical composition are each administered at the same dose of CAR T cells/kg as administered in the first dose of the pharmaceutical composition. In some embodiments, the second dose of the pharmaceutical composition is administered at the same dose of CAR T cells/kg as administered in the first dose of the pharmaceutical composition, and the third dose of the pharmaceutical composition is administered at a different dose of CAR T cells/kg than administered in the first dose of the pharmaceutical composition. In some embodiments, the second dose of the pharmaceutical composition is administered at a different dose of CAR T cells/kg than administered in the first dose of the pharmaceutical composition, and the third dose of the pharmaceutical composition is administered at the same dose of CAR T cells/kg as administered in the first dose of the pharmaceutical composition. In some embodiments, the second dose of the pharmaceutical composition is administered at a different dose of CAR T cells/kg than administered in the first dose of the pharmaceutical composition, and the third dose of the pharmaceutical composition is administered at the same dose of CAR T cells/kg as administered in the first dose of the pharmaceutical composition. In some embodiments, the second dose of the pharmaceutical composition and the third dose of the pharmaceutical composition are administered at the same dose of CAR T cells/kg, and are administered at a different dose of CAR T cells/kg than administered in the first dose of the pharmaceutical composition.

In some embodiments, the method comprises re-administration of the lymphodepletion regimen and the pharmaceutical composition to the subject. In some embodiments, re-administration of the lymphodepletion regimen and the pharmaceutical composition occurs following a partial response or complete response to the first lymphodepletion regimen and pharmaceutical composition with subsequent progressive disease. In some embodiments, re-administration of the lymphodepletion regimen and the pharmaceutical composition occurs following no response to the first lymphodepletion regimen and pharmaceutical composition and subsequent progressive disease. In some embodiments, re-administration of the lymphodepletion regimen and the pharmaceutical composition occurs in subjects having a cancer that remains positive for the cancer cell antigen targeted by the CAR T cells (e.g., are positive for CD19, CD20, or BCMA). In some embodiments, re-administration of the lymphodepletion regimen and the pharmaceutical composition occurs about 2 weeks, 4 weeks, 6, weeks, 8 weeks, 10 weeks, 12, weeks, 14, weeks, 16 weeks, 18 weeks, 20 weeks, 22 weeks, 24 weeks, or more after the first administration of the lymphodepletion regimen and pharmaceutical composition. In some embodiments, the lymphodepletion regimen is re-administered at the same doses and/or schedule as the first administration. In some embodiments, the lymphodepletion regimen is re-administered at different doses and/or a different schedule as the first administration. In some embodiments, the pharmaceutical composition is re-administered at the same doses and/or schedule as the first administration. In some embodiments, the pharmaceutical composition is re-administered at different doses and/or a different schedule as the first administration. In certain embodiments, the pharmaceutical composition is re-administered at a higher dose than the first administration. In some embodiments, the pharmaceutical composition is re-administered at a dose of about 1×10⁶ CAR T cells/kg. In some embodiments, the pharmaceutical composition is re-administered at a dose of about 2×10⁶ CAR T cells/kg. In some embodiments, the pharmaceutical composition is re-administered at a dose of about 3×10⁶ CAR T cells/kg. In some embodiments, the pharmaceutical composition is re-administered at a dose of about 6×10⁶ CAR T cells/kg. In some embodiments, a first dose and a second dose, and optionally a third dose, of the pharmaceutical composition are re-administered according to any of the doses and dosing schedules described herein for administration of a first dose and a second dose, or for administration of a first dose, a second dose, and a third dose.

In some embodiments, the human T cells are not derived from the subject (e.g., the human T cells are allogeneic).

In some embodiments, the cancer is a cancer of B cell origin or multiple myeloma. In some such embodiments, the cancer of B cell origin is acute lymphoblastic leukemia (ALL), chronic lymphocytic leukemia (CLL), small lymphocytic lymphoma (SLL), or non-Hodgkin lymphoma (NHL). In some embodiments, the cancer is Relapsed/Refractory (R/R) NHL. Relapsed/Refractory (R/R) ALL. In other such embodiments, the cancer is mantle cell lymphoma (MCL) or diffuse large B cell lymphoma (DLBCL).

In some embodiments, the subject is refractory to prior CAR T immunotherapy.

In some embodiments, administration of the lymphodepletion regimen in combination with any of the disclosed pharmaceutical compositions, or any of the disclosed methods of immunotherapy, results in the prevention, either partially or completely, of the occurrence of graft versus host disease (GvHD). In some embodiments, administration of any of the disclosed pharmaceutical compositions, or any of the disclosed methods of immunotherapy results in an achievement of a partial response or a complete response to the immunotherapy. In some embodiments, the partial response or the complete response is maintained through at least 28 days after administration of the pharmaceutical composition.

In some embodiments, the lymphodepletion regimen includes administration of a biological lymphodepletion agent in an amount no greater than 1.0 mg/kg during the 7 day period preceding administration of the pharmaceutical composition. In some embodiments, the lymphodepletion regimen includes administration of a biological lymphodepletion agent in an amount no greater than 0.75 mg/kg, 0.5 mg/kg, 0.25 mg/kg, or 0.1 mg/kg during the 7 day period preceding administration of the pharmaceutical composition. In certain embodiments, the lymphodepletion regimen includes administration of a biological lymphodepletion agent in an amount no greater than 0.1 mg/kg during the 7 day period preceding administration of the pharmaceutical composition.

In some embodiments, the biological lymphodepletion agent of the described regimens is a monoclonal antibody, or a fragment thereof. In some such embodiments, the monoclonal antibody, or fragment thereof, has specificity for a T cell antigen. In certain embodiments, the monoclonal antibody, or fragment thereof, is an anti-CD52 monoclonal antibody, or fragment thereof, or an anti-CD3 antibody, or fragment thereof. In particular embodiments, the monoclonal antibody is alemtuzumab or ALLO-647.

In some embodiments, the one or more chemotherapeutic agents of the described regimens comprises cyclophosphamide and fludarabine. In some such embodiments, the lymphodepletion regimen comprises administering cyclophosphamide at a dose of about 400 mg/m²/day to about 1500 mg/m²/day. In some embodiments, the lymphodepletion regimen comprises administering cyclophosphamide at a dose of about 500 mg/m²/day to about 1000 mg/m²/day. In particular embodiments, the lymphodepletion regimen comprises administering cyclophosphamide at a dose of about 500 mg/m²/day. In particular embodiments, the lymphodepletion regimen comprises administering cyclophosphamide at a dose of between about 500 mg/m²/day and 1500 mg/m²/day. In some embodiments, the lymphodepletion regimen comprises administering fludarabine at a dose of about 25 mg/m²/day to about 40 mg/m²/day. In particular embodiments, the lymphodepletion regimen comprises administering fludarabine at a dose of about 30 mg/m²/day.

In certain embodiments, the lymphodepletion regimen comprises administering cyclophosphamide at a dose of about 500 mg/m²/day and fludarabine at a dose of about 30 mg/m²/day. In other embodiments, the lymphodepletion regimen comprises administering cyclophosphamide at a dose of between about 500 mg/m²/day and 1500 mg/m²/day and fludarabine at a dose of about 30 mg/m²/day.

In some embodiments, the lymphodepletion regimen is administered to the subject once daily starting 5 days and ending 3 days or 2 days prior to administration of the pharmaceutical composition. In other embodiments, the lymphodepletion regimen is administered to the subject once daily for at least one day, or for multiple days, within 7 days prior to administration of the pharmaceutical composition.

In some embodiments, the lymphodepletion regimen comprises administering cyclophosphamide at a dose of about 1000 mg/m²/day. In some embodiments, the lymphodepletion regimen comprises administering cyclophosphamide at a dose of about 1000 mg/m²/day and fludarabine at a dose of about 30 mg/m²/day. In some embodiments, the lymphodepletion regimen is administered to the subject once daily for at least one day, or for multiple days, within 7 days prior to administration of the pharmaceutical composition. In some embodiments, the lymphodepletion regimen comprises administering fludarabine at a dose of about 30 mg/m²/day for three days, e.g., starting 6 days and ending 3 days prior to administration of the pharmaceutical composition. In some embodiments, the lymphodepletion regimen comprises administering cyclophosphamide at a dose of about 1000 mg/m²/day for two days, e.g., starting 5 days and ending 3 days prior to administration of the pharmaceutical composition. In some embodiments, the lymphodepletion regimen comprises administering cyclophosphamide once daily starting 5 days and ending 3 days prior to administration of the pharmaceutical composition, and administering fludarabine once daily starting 6 days and ending 3 days prior to administration of the pharmaceutical composition.

In some embodiments, the pharmaceutical composition is administered at a dose of between about 3×10⁴ and about 6×10⁶ CAR T cells/kg. In some embodiments, the pharmaceutical composition is administered at a dose of between about 1×10⁵ and about 6×10⁶ CAR T cells/kg. In certain embodiments, the pharmaceutical composition is administered at a dose of between about 3×10⁵ and about 6×10⁶ CAR T cells/kg. In particular embodiments, the pharmaceutical composition is administered at a dose of between about 3×10⁵ and about 3×10⁶ CAR T cells/kg. In particular embodiments, the pharmaceutical composition is administered at a dose of between about 6×10⁵ and about 6×10⁶ CAR T cells/kg. In certain embodiments, the pharmaceutical composition is administered at a dose of about 3×10⁴ CAR T cells/kg. In certain embodiments, the pharmaceutical composition is administered at a dose of about 3×10⁵ CAR T cells/kg. In certain embodiments, the pharmaceutical composition is administered at a dose of about 6×10⁵ CAR T cells/kg. In certain embodiments, the pharmaceutical composition is administered at a dose of about 1×10⁶ CAR T cells/kg. In certain embodiments, the pharmaceutical composition is administered at a dose of about 2×10⁶ CAR T cells/kg. In certain embodiments, the pharmaceutical composition is administered at a dose of about 3×10⁶ CAR T cells/kg. In certain embodiments, the pharmaceutical composition is administered at a dose of about 6×10⁶ CAR T cells/kg.

In some embodiments, wherein a second dose of the pharmaceutical composition is administered, the first dose of the pharmaceutical composition is administered at a dose of between about 5×10⁵ to about 3×10⁶ CAR T cells/kg and the second dose of the pharmaceutical composition is administered at a dose of between about 5×10⁵ to about 3×10⁶ CAR T cells/kg, such that a total number of between about 1×10⁶ to about 6×10⁶ CAR T cells/kg are administered to the subject. In some embodiments, the lymphodepletion regimen is not re-administered prior to administration of the second dose of the pharmaceutical composition.

In some embodiments, wherein a second dose of the pharmaceutical composition is administered, the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg are administered to the subject. In some embodiments, the lymphodepletion regimen is not re-administered prior to administration of the second dose of the pharmaceutical composition.

In some embodiments, wherein a second dose of the pharmaceutical composition and a third dose of the pharmaceutical composition are administered, the first dose of the pharmaceutical composition is administered at a dose of between about 0.33×10⁵ to about 3×10⁶ CAR T cells/kg, the second dose of the pharmaceutical composition is administered at a dose of between about 0.33×10⁵ to about 3×10⁶ CAR T cells/kg, and the third dose of the pharmaceutical composition is administered at a dose of between about 0.33×10⁵ to about 3×10⁶ CAR T cells/kg, such that a total number of between about 1×10⁶ to about 9×10⁶ CAR T cells/kg are administered to the subject. In some embodiments, the lymphodepletion regimen is not re-administered prior to administration of the second dose of the pharmaceutical composition or the third dose of the pharmaceutical composition.

In some embodiments, wherein a second dose of the pharmaceutical composition and a third dose of the pharmaceutical composition are administered, the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg are administered to the subject. In some embodiments, the lymphodepletion regimen is not re-administered prior to administration of the second dose of the pharmaceutical composition or the third dose of the pharmaceutical composition.

In some embodiments, wherein a second dose of the pharmaceutical composition and a third dose of the pharmaceutical composition are administered, the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg are administered to the subject. In some embodiments, the lymphodepletion regimen is not re-administered prior to administration of the second dose of the pharmaceutical composition or the third dose of the pharmaceutical composition.

In some embodiments, a transgene encoding the CAR is inserted into the genome of the CAR T cells within the TCR alpha gene or the TCR beta gene, wherein the transgene disrupts expression of the TCR alpha gene or the TCR beta gene. In certain embodiments, the transgene encoding the CAR is inserted into a TCR alpha constant region (i.e., TRAC) gene. In particular embodiments, the transgene encoding the CAR is inserted into an engineered meganuclease recognition sequence comprising SEQ ID NO: 1. In such embodiments, the transgene encoding the CAR can be inserted between positions 13 and 14 of SEQ ID NO: 1.

In some embodiments, the CAR T cells do not have detectable cell surface expression of an endogenous alpha/beta TCR. In some embodiments, the CAR T cells do not have detectable cell surface expression of CD3.

In some embodiments, the extracellular ligand-binding domain is a single-chain variable fragment (scFv).

In some embodiments, the extracellular ligand-binding domain has specificity for CD19, CD20, or b cell maturation antigen (BCMA; i.e., CD269). In certain embodiments, the extracellular ligand-binding domain has specificity for CD19. In certain embodiments, the extracellular ligand-binding domain has specificity for CD20. In certain embodiments, the extracellular ligand-binding domain has specificity for BCMA.

In certain embodiments, the extracellular ligand-binding domain is a single-chain variable fragment (scFv) comprising: (a) a heavy chain variable domain (VH) of SEQ ID NO: 3 and a light chain variable domain (VL) of SEQ ID NO: 4; or (b) a heavy chain variable domain (VH) of SEQ ID NO: 6 and a light chain variable domain (VL) of SEQ ID NO: 7.

In some embodiments, the hinge domain is a CD8 alpha hinge domain. In some embodiments, the transmembrane domain is a CD8 alpha transmembrane domain. In some embodiments, the co-stimulatory domain comprises one or more TRAF-binding domains. In certain embodiments, the co-stimulatory domain comprises a first domain comprising SEQ ID NO: 9 and a second domain comprising SEQ ID NO: 10 or 11. In particular embodiments, the co-stimulatory domain is a novel 6 (N6) co-stimulatory domain or a 4-1BB co-stimulatory domain. In certain embodiments, the co-stimulatory domain in an N6 co-stimulatory domain. In some embodiments, the co-stimulatory domain is a 4-1BB co-stimulatory domain. In some embodiments, the intracellular signaling domain is a CD3 zeta domain.

In some embodiments, the CAR comprises an amino acid sequence having at least 80%, at least 85%, at least 90%, at least 95%, or more, sequence identity to SEQ ID NO: 5 and has specificity for CD19, or an amino acid sequence having at least 80%, at least 85%, at least 90%, at least 95%, or more, sequence identity to SEQ ID NO: 8 and has specificity for CD20. In certain embodiments, the CAR comprises an amino acid sequence of SEQ ID NO: 5 or 8. In certain embodiments, the CAR comprises an amino acid sequence having at least 80%, at least 85%, at least 90%, at least 95%, or more, sequence identity to SEQ ID NO: 5 and has specificity for CD19. In certain embodiments, the CAR comprises an amino acid sequence having at least 80%, at least 85%, at least 90%, at least 95%, or more, sequence identity to SEQ ID NO: 8 and has specificity for CD20.

In some embodiments, the CAR T cells represent between about 50% and about 70% of the human T cells in the population. In certain embodiments, the CAR T cells represent between about 55% and about 70% of the human T cells in the population. In particular embodiments, the CAR T cells represent between about 58% and about 69% of the human T cells in the population. In some embodiments, the CAR T cells represent greater than about 70% of the human T cells in the population. In some embodiments, the CAR T cells represent greater than about 80% of the human T cells in the population.

In some embodiments, no more than about 0.5% of the human T cells in the population have detectable cell surface expression of CD3. In certain embodiments, no more than about 0.3% of the human T cells in the population have detectable cell surface expression of CD3. In certain embodiments, no more than about 0.2% of the human T cells in the population have detectable cell surface expression of CD3. In certain embodiments, no human T cells in the population have detectable cell surface expression of CD3.

In some embodiments, the ratio of CD4+ CAR T cells to CD8+ CAR T cells in the population (CD4:CD8 ratio, or CD4+/CD8+ cell ratio) is between about 0.7 and about 2.5. In certain embodiments, the ratio of CD4+ CAR T cells to CD8+ CAR T cells in the population is greater than about 2.5.

In some embodiments, the percentage of CD4+ CAR T cells in the population that are also CCR7+ is (CD4+/CCR7+) between about 35% to about 70%. In certain embodiments, the percentage of CD4+ CAR T cells in the population that are also CCR7+ is between about 38% to about 68%. In certain embodiments, the percentage of CD4+ CAR T cells in the population that are also CCR7+ is between about 39% to about 69%. In certain embodiments, the percentage of CD4+ CAR T cells in the population that are also CCR7+ is between about 40% to about 68%. In some embodiments, the percentage of CD4+ CAR T cells in the population that are also CCR7+ is greater than about 70%. In some embodiments, the percentage of CD4+ CAR T cells in the population that are also CCR7+ is greater than about 80%.

In some embodiments, the percentage of CD8+ CAR T cells in the population that are also CCR7+(CD8+/CCR7+) is between about 25% and about 45%. In certain embodiments, the percentage of CD8+ CAR T cells in the population that are also CCR7+ is between about 31% and about 42%. In certain embodiments, the percentage of CD8+ CAR T cells in the population that are also CCR7+ is between about 30% and about 42%. In certain embodiments, the percentage of CD8+ CAR T cells in the population that are also CCR7+ is between about 30% and about 45%. In some embodiments, the percentage of CD8+ CAR T cells in the population that are also CCR7+ is greater than about 45%. In some embodiments, the percentage of CD8+ CAR T cells in the population that are also CCR7+ is greater than about 50%.

In some embodiments, the CAR T cells proliferate in vivo for at least one day following administration of the pharmaceutical composition. In certain embodiments, the CAR T cells proliferate in vivo between about day 1 and about day 21 following administration of the pharmaceutical composition. In certain embodiments, the number of copies of the CAR transgene per 14 of DNA in peripheral blood mononuclear cells is elevated for at least 1 day, and for up to at least 21 days after administration of the pharmaceutical composition when compared to the number of transgene copies per μg of DNA in peripheral blood mononuclear cells present prior to administration. In particular embodiments, the number of copies of the CAR transgene per μg of DNA in peripheral blood mononuclear cells is elevated to between about 150 copies/14 to about 2100 copies/μg of DNA for at least 1 day following administration of the pharmaceutical composition.

In some embodiments, the serum concentration of C-reactive protein, ferritin, IL-6, interferon gamma, or any combination thereof, is elevated compared to the concentration at day 0 for at least 1 day following administration of the pharmaceutical composition.

In some embodiments, the subject achieves a partial response or a complete response to the method of immunotherapy. In certain embodiments, the partial response or the complete response is maintained through at least 28 days after administration of the pharmaceutical composition. In certain embodiments, the partial response or the complete response is maintained through at least 30 days, at least 35 days, at least 40 days, or more than 40 days after administration of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 55% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45% (i.e., the percentage of CD3-CD4+ CCR7+ is between about 35% and about 70%, and the percentage of CD3-CD8+ CCR7+ is between about 25% and about 45%). In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain, wherein the CAR T cells represent between about 58% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the dose of the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 55% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 3×10⁴ CAR T cells/kg. In some embodiments, pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain, wherein the CAR T cells represent between about 58% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 55% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 3×10⁴ CAR T cells/kg. In some embodiments, pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is ALL, MCL, or DLBCL, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain, wherein the CAR T cells represent between about 58% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the pharmaceutical composition is administered at a dose of about 3×10³ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered 10 days following the administration of the first dose of the pharmaceutical composition, wherein the first dose of the pharmaceutical composition is administered at a dose of about 3×10⁶ CAR T cells/kg and the second dose of the pharmaceutical composition is administered at a dose of about 3×10⁶ CAR T cells/kg, such that a total number of about 6×10⁶ CAR T cells/kg is administered to the subject. In some embodiments, the lymphodepletion regimen is not re-administered prior to administration of the second dose of the pharmaceutical composition. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 1×10⁶ CAR T cells/kg, the second dose of the pharmaceutical composition is administered at a dose of about 1×10⁶ CAR T cells/kg, and the third dose of the pharmaceutical composition is administered at a dose of about 1×10⁶ CAR T cells/kg, such that a total number of about 3×10⁶ CAR T cells/kg is administered to the subject. In some particular embodiments, a second dose of the pharmaceutical composition is administered 10 days following the administration of the first dose of the pharmaceutical composition, and a third dose of the pharmaceutical composition is administered 4 days following the administration of the second dose of the pharmaceutical composition, wherein the first dose of the pharmaceutical composition is administered at a dose of about 3×10⁶ CAR T cells/kg, the second dose of the pharmaceutical composition is administered at a dose of about 3×10⁶ CAR T cells/kg, and the third dose of the pharmaceutical composition is administered at a dose of about 3×10⁶ CAR T cells/kg, such that a total number of about 9×10⁶ CAR T cells/kg is administered to the subject. In some embodiments, the lymphodepletion regimen is not re-administered prior to administration of the second dose of the pharmaceutical composition and the third dose of the pharmaceutical composition.

In some embodiments, the cancer is NHL, CLL, or SLL, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is NHL, CLL, or SLL, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is NHL, CLL, or SLL, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 3×10⁴ CAR T cells/kg. In some embodiments, pharmaceutical composition is administered at a dose of about 3×10⁵ CAR T cells/kg. In some embodiments, the pharmaceutical composition is administered at a dose of about 1×10⁶ CAR T cells/kg. In some embodiments, pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is NHL, CLL, or SLL, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is NHL, CLL, or SLL, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 3×10⁴ CAR T cells/kg. In some embodiments, pharmaceutical composition is administered at a dose of about 3×10⁵ CAR T cells/kg. In some embodiments, the pharmaceutical composition is administered at a dose of about 1×10⁶ CAR T cells/kg. In some embodiments, pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is NEIL, CLL, or SLL, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 3×10⁴ and 3×10⁶ CAR T cells/kg, wherein the transgene encoding the CAR is inserted into a TCR alpha constant region gene, wherein the CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 3×10⁴ CAR T cells/kg. In some embodiments, the pharmaceutical composition is administered at a dose of about 3×10⁵ CAR T cells/kg. In some embodiments, the pharmaceutical composition is administered at a dose of about 1×10⁶ CAR T cells/kg. In some embodiments, the pharmaceutical composition is administered at a dose of about 3×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of about 500 mg/m²/day and fludarabine at a dose of about 30 mg/m²/day, wherein the lymphodepletion regimen is administered to the subject once daily starting 5 days and ending 3 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of between about 500 and 1500 mg/m²/day and fludarabine at a dose of about 30 mg/m²/day, wherein the lymphodepletion regimen is administered to the subject once daily for at least one day, or for multiple days, within 7 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain, wherein the CAR T cells represent between about 60% and about 70% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 35% to about 70%, and wherein the percentage of CD8+ CAR T cells in the population that are also CCR7+ is between about 25% and about 45%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the cancer is multiple myeloma, wherein the biological lymphodepletion agent is an anti-CD52 monoclonal antibody, wherein the lymphodepletion regimen comprises administering cyclophosphamide at a dose of about 1000 mg/m²/day and fludarabine at a dose of about 30 mg/m²/day, wherein the cyclophosphamide is administered to the subject once daily starting 5 days and ending 3 days prior to administration of the pharmaceutical composition, and the fludarabine is administered to the subject once daily starting 6 days and ending 3 days prior to administration of the pharmaceutical composition, wherein the pharmaceutical composition is administered at a dose of between about 6×10⁵ and 6×10⁶ CAR T cells/kg, wherein the transgene encoding the CAR is inserted into a TCR alpha constant region gene, wherein the CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain, wherein the CAR T cells represent between about 64% and about 69% of the human T cells in the population, wherein no more than about 0.3% of the human T cells in the population have detectable cell surface expression of CD3, wherein the ratio of CD4+ CAR T cells to CD8+ CAR T cells in the population is between about 0.7 and about 2.5, wherein the percentage of CD4+ CAR T cells in the population that are also CCR7+ is between about 40% to about 68%, and wherein the percentage of CD8+ CAR T cells in the population that are also CCR7+ is between about 31% and about 42%. In some embodiments, the pharmaceutical composition is administered at a dose of about 6×10⁵ CAR T cells/kg. In some embodiments, the pharmaceutical composition is administered at a dose of about 2×10⁶ CAR T cells/kg. In some embodiments, the pharmaceutical composition is administered at a dose of about 6×10⁶ CAR T cells/kg. In some particular embodiments, a second dose of the pharmaceutical composition is administered following the administration of the first dose of the pharmaceutical composition and without re-administration the lymphodepletion regimen. In some particular embodiments, a second dose of the pharmaceutical composition and a third dose of the pharmaceutical composition are administered following the administration of the first dose of the pharmaceutical composition and without re-administration of the lymphodepletion regimen.

In some embodiments, the method further comprises manufacturing the population of human T cells, wherein the manufacturing comprises: (a) a first culturing step wherein isolated human T cells are cultured in media for 3 days with anti-CD3 and anti-CD28 antibodies bound to a matrix or particle; (b) electroporating the isolated human T cells to introduce mRNA encoding an engineered nuclease having specificity for a recognition sequence within the TCR alpha gene, wherein the engineered nuclease is expressed in the human T cells and generates a cleavage site at the recognition sequence; (c) transducing the isolated human T cells with a recombinant AAV vector comprising a donor template, wherein the donor template comprises a transgene encoding the CAR, and wherein the donor template is flanked by a 5′ homology arm having homology to sequences 5′ upstream of the cleavage site, and by a 3′ homology arm having homology to sequences 3′ downstream of the cleavage site, wherein the donor template is inserted into the genome of the isolated human T cells at the cleavage site; (d) a second culturing step wherein the isolated human T cells are cultured in media for about 5 days; (e) removing the isolated human T cells that express cell surface CD3 using anti-CD3 antibodies; and (f) a third culturing step wherein the isolated human T cells are cultured in media for about 2 days to generate the population of human T cells.

In some embodiments, the method further comprises a step of concentrating the population of human T cells after the third culturing step.

In some embodiments, the method further comprises a step of formulating the population of human T cells in cryopreservation media after the concentrating.

In some embodiments, the manufacturing is completed in about 10 days or less.

In some embodiments, the anti-CD3 and anti-CD28 antibodies are bound to beads.

In some embodiments, the anti-CD3 antibodies are conjugated to magnetic beads.

In some embodiments, the recombinant AAV vector has a serotype of AAV6.

In some embodiments, the engineered nuclease is an engineered meganuclease, a zinc finger nuclease, a TALEN, a compact TALEN, a CRISPR system nuclease, or a megaTAL. In certain embodiments, the engineered nuclease is an engineered meganuclease. In particular embodiments, the engineered meganuclease has specificity for a recognition sequence comprising SEQ ID NO: 1. In particular embodiments, the engineered meganuclease comprises an amino acid sequence of SEQ ID NO: 19.

In another aspect, the invention provides a method of immunotherapy for treating cancer in a subject, the method comprising: (a) administering to the subject a lymphodepletion regimen comprising one or more chemotherapeutic lymphodepletion agents; and (b) administering to the subject effective amounts of a first dose, a second dose, and optionally a third dose of a pharmaceutical composition without re-administration of the lymphodepletion regimen, wherein the pharmaceutical composition comprises a population of human T cells, wherein a plurality of the human T cells are chimeric antigen receptor (CAR) T cells expressing a cell surface CAR, wherein a T cell receptor (TCR) alpha gene or a TCR beta gene is inactivated in the CAR T cells, and wherein the pharmaceutical composition is administered at a dose of between about 3×10⁴ to about 1×10⁷ CAR T cells/kg; wherein the lymphodepletion regimen is administered prior to administration of the first dose of the pharmaceutical composition, and wherein the CAR comprises an extracellular ligand-binding domain having specificity for a cancer cell antigen, a hinge domain, a transmembrane domain, a co-stimulatory domain, and an intracellular signaling domain.

In some embodiments, the lymphodepletion regimen can be any lymphodepletion regimen described herein.

In some embodiments, the pharmaceutical composition can be any pharmaceutical composition described herein.

In some such embodiments, the method comprises administering the second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition. In certain embodiments, the method comprises administering a second dose of the pharmaceutical composition 10 days following administration of the first dose of the pharmaceutical composition. In some embodiments, the second dose of the pharmaceutical composition is administered at the same dose of CAR T cells/kg as administered in the first dose of the pharmaceutical composition. In some embodiments, the second dose of the pharmaceutical composition is administered at a different dose of CAR T cells/kg than administered in the first dose of the pharmaceutical composition.

In some such embodiments, the method comprises administering the second dose of the pharmaceutical composition 1, 2, 3, 4, 5, 6, 7, 8, 9, 10, 11, 12, 13, 14, 15, 16, 17, 18, 19, 20, or 21 days following administration of the first dose of the pharmaceutical composition, and further comprises administering the third dose of the pharmaceutical composition 1, 2, 3, 4, 5, 6, 7, 8, 9, 10, 11, 12, 13, 14, 15, 16, 17, 18, 19, 20, or 21 days following administration of the second dose of the pharmaceutical composition. In some embodiments, the second dose of the pharmaceutical composition is administered 10 days following administration of the first dose of the pharmaceutical composition, and the third dose of the pharmaceutical composition is administered 4 days following administration of the second dose of the pharmaceutical composition. In some embodiments, the second dose of the pharmaceutical composition and the third dose of the pharmaceutical composition are each administered at the same dose of CAR T cells/kg as administered in the first dose of the pharmaceutical composition. In some embodiments, the second dose of the pharmaceutical composition is administered at the same dose of CAR T cells/kg as administered in the first dose of the pharmaceutical composition, and the third dose of the pharmaceutical composition is administered at a different dose of CAR T cells/kg than administered in the first dose of the pharmaceutical composition. In some embodiments, the second dose of the pharmaceutical composition is administered at a different dose of CAR T cells/kg than administered in the first dose of the pharmaceutical composition, and the third dose of the pharmaceutical composition is administered at the same dose of CAR T cells/kg as administered in the first dose of the pharmaceutical composition. In some embodiments, the second dose of the pharmaceutical composition and the third dose of the pharmaceutical composition are administered at the same dose of CAR T cells/kg, and are administered at a different dose of CAR T cells/kg than administered in the first dose of the pharmaceutical composition.

### BRIEF DESCRIPTION OF THE FIGURES

FIG. 1 shows the PBCAR0191 study design. In this example of the study design, lymphodepletion includes cyclophosphamide administered once daily at a dose of 500 mg/m² and fludarabine administered once daily at a dose of 30 mg/m² beginning on day −5 and ending on day −3. The study design can be modified as described herein, for example, to include split dosing of the CAR T cells, different lengths of time and different doses for lymphodepletion, and repeated lymphodepletion and dosing.

FIG. 2 shows characteristics of five clinical trial material (CTM) batches used for dosing in the PBCAR0191 study.

FIG. 3 shows PBCAR0191 study methods, endpoints, and objectives.

FIG. 4 shows PBCAR0191 select inclusion and exclusion criteria.

FIG. 5 shows PBCAR0191 patient demographics.

FIG. 6 shows PBCAR0191 Grade 3 or higher treatment emergent adverse events (cohorts N and A).

FIG. 7 shows PBCAR0191 summary of adverse events related to lymphodepletion.

FIGS. 8A-8B show PBCAR0191 non-Hodgkin lymphoma cohort baseline characteristics, prior treatments, prognostic indicators, and outcomes.

FIGS. 9A-9B show PBCAR0191 acute lymphoblastic leukemia cohort baseline characteristics, prior treatments, prognostic indicators, and outcomes.

FIG. 10 shows a summary of PBCAR0191 objective responses.

FIGS. 11A-11C show serum cytokine responses, B cell aplasia, and CAR T cell expansion in Patient 3-NHL-DL1. FIG. 11A shows serum cytokine responses. CRP (#), ferritin (*), IL-6 (+), IL-I5 (=) and IFN-g (\hat{ }) are indicated with respective symbols. FIG. 11B shows B cell aplasia. FIG. 11C shows CAR T cell expansion.

FIG. 12 shows PET scans of Patient 3-NHL-DL1.

FIGS. 13A-13C show serum cytokine responses, B cell aplasia, and CAR T cell expansion in Patient 4-NHL-DL2. FIG. 13A shows serum cytokine responses. CRP (#), ferritin (*), IL-6 (+), IL-15 (=) and IFN-g (\hat{ }) are indicated with respective symbols. FIG. 13B shows B cell aplasia. FIG. 13C shows CAR T cell expansion.

FIG. 14 shows PET scans of Patient 4-NHL-DL2.

FIG. 15 shows CAR T cell expansion in multiple patients in the NHL cohort at dose level 1 and dose level 2. 1-NHL-DL1 ({); 2-NHL-DL1 (*); 3-NHL-DL1 (+); 4-NHL-DL2 (=); 5-NHL-DL2 (\hat{ }); and 6-NHL-DL2, 7-ALL-DL2, 8-ALL-DL2, 9-ALL-DL2 (@) are indicated with respective symbols.

FIGS. 16A-16B show serum cytokine responses and B cell aplasia in Patient 6-NHL-DL2. FIG. 16A shows serum cytokine responses. CRP (#), ferritin (*), IL-6 (+), IL-15 (=) and IFN-g (\hat{ }) are indicated with respective symbols. FIG. 16B shows B cell aplasia.

FIG. 17 shows PET scans of Patient 6-NHL-DL2.

FIGS. 18A-18B show serum cytokine responses and bone marrow analysis in Patient 9-ALL-DL2. FIG. 18A shows serum cytokine responses. CRP (#), ferritin (*), IL-6 (+), IL-15 (=) and IFN-g (\hat{ }) are indicated with respective symbols. FIG. 18B shows bone marrow analysis at baseline and day 28 following administration of PBCAR0191.

FIG. 19 shows pre-PBCAR0191 aspirate flow analysis for B-ALL blasts in Patient 9-ALL-DL2. Arrows note the presence of tumor cells (+), markers for intact cells (\hat{ }) and non-tumor cells of hematopoietic origin (=).

FIG. 20 shows day 28 PBCAR0191 aspirate flow analysis for B-ALL blasts in Patient 9-ALL-DL2.

FIG. 21 shows PBCAR0191 study conclusions.

FIG. 22 shows the PBCAR20A study design. In this example of the study design, lymphodepletion includes cyclophosphamide administered once daily at a dose of 500 mg/m² and fludarabine administered once daily at a dose of 30 mg/m² beginning on day −5 and ending on day −3 (for a total dose of 1500 mg/m² of cyclophosphamide and a total dose of 90 mg/m² of fludarabine). The study design can be modified as described herein, for example, to include split dosing of the CAR T cells, different lengths of time and different doses for lymphodepletion, and repeated lymphodepletion and dosing.

FIG. 23 shows the PBCAR269A study design. In this example of the study design, lymphodepletion includes cyclophosphamide administered once daily at a dose of 500 mg/m² and fludarabine administered once daily at a dose of 30 mg/m² beginning on day −5 and ending on day −3 (for a total dose of 1500 mg/m² of cyclophosphamide and a total dose of 90 mg/m² of fludarabine). The study design can be modified as described herein, for example, to include split dosing of the CAR T cells, different lengths of time and different doses for lymphodepletion, and repeated lymphodepletion and dosing.

FIGS. 24A-24B show dose-dependent CAR T cell expansion. Results show dose-dependent CAR T cell expansion in first two weeks after administration followed by decline. Cell counts were determined by flow cytometry. FIG. 24A shows CAR T cell expansion in NHL patients. Dose levels DL1 (+), DL2 (=) and DL3 (\hat{ }) are indicated with respective symbols. FIG. 24B shows CAR T cell expansion in B-ALL patients. Dose levels DL2 (=) and DL3 (\hat{ }) are indicated with respective symbols.

FIG. 25 shows patient CD3 counts rise rapidly following CAR T expansion. The demonstrated increase in CD3+ cells following CAR T cell expansion is consistent with alloreactive rejection. Results show patients' T cells rise quickly following CAR T expansion, suggesting that they are expanding in response to the CAR T cells and driving rejection. Dose levels DL1 (+), DL2 (=) and DL3 (\hat{ }), and CD3+ cells (#) are indicated with respective symbols.

FIG. 26 shows split dosing of Dose Level 4 (DL4), or two doses of 3×10⁶ cells/kg body weight. Results show no evidence of cell expansion or improved response after a second infusion in 3 patients (split-dose DL4). DL4 therefore considered equivalent to dose level 3 (DL3) (3×10⁶ cells/kg). The tumor burden and previous lines of therapy for this patient are indicated. CAR T cells/ml (=) and CD3+ cells/ml (\hat{ }) are indicated with respective symbols.

FIG. 27 shows repeat dosing in a patient. Results show expansion of CAR T cells following a second LD administration. The second LD administration enables cell expansion following a repeat CAR T dose and effectively increased the “area under the curve” (AUC) to circumvent rejection. The tumor burden and previous lines of therapy for this patient are indicated. CAR T cells/ml (=) and CD3+ cells/ml (\hat{ }) are indicated with respective symbols.

FIG. 28 shows enhanced LD increased peak CAR T expansion and the AUC by a factor of greater than about 95× and about 45×, respectively, relative to standard lymphodepletion regiment (sLD, indicated by (+)). Increased cell expansion with enhanced lymphodepletion regimen (eLD, indicated by (=)) correlated with a 100% overall response rate (ORR) (75% complete response (CR) rate) in NHL patients without prolonged cytopenia. No dose limiting toxicities were observed.

FIG. 29 shows enhanced LD increased peak CAR T expansion about 95× vs sLD. Increased cell expansion with eLD correlated with a 100% ORR (75% CR rate) in NHL patients without prolonged cytopenia. No dose limiting toxicities. Enhanced LD enabled higher CAR T expansion without long-term immunosuppression. sLD+DL3 (+), eLD+DL3 (+), CD3+sLD+DL3 (#), CD3+eLD+DL3 (\hat{ }) are indicated with respective symbols.

FIG. 30 shows the durability of the responses in NHL patients following administration of split dose and enhanced LD regimens.

FIG. 31 shows the durability of the responses in B-ALL patients following administration of split dose and enhanced LD regimens.

FIG. 32 shows the best percent change in tumor area in patients treated with standard LD and enhanced LD.

FIG. 33 shows that dose level 3 (DL3) administration enhanced the level of lymphodepletion in a patient. Previous lines of therapy for this patient are indicated. CAR T/ml (+), CD3+ cells/ml (=), CD4+CD3+ cells/ml (#), CD8+CD3+ cells/ml (\hat{ }) are indicated with respective symbols.

### BRIEF DESCRIPTION OF THE SEQUENCES

SEQ ID NO: 1 sets forth the nucleic acid sequence of the TRC 1-2 recognition sequence (sense).

SEQ ID NO: 2 sets forth the nucleic acid sequence of the TRC 1-2 recognition sequence (antisense).

SEQ ID NO: 3 sets forth the amino acid sequence of the heavy chain variable region of a murine anti-CD19 antibody.

SEQ ID NO: 4 sets forth the amino acid sequence of the light chain variable region of a murine anti-CD19 antibody.

SEQ ID NO: 5 sets forth the amino acid sequence of an anti-CD19 chimeric antigen receptor.

SEQ ID NO: 6 sets forth the amino acid sequence of the heavy chain variable region of a murine anti-CD20 antibody.

SEQ ID NO: 7 sets forth the amino acid sequence of the light chain variable region of a murine anti-CD20 antibody.

SEQ ID NO: 8 sets forth the amino acid sequence of an anti-CD20 chimeric antigen receptor.

SEQ ID NO: 9 sets forth the amino acid sequence of a domain found in a TRAF-binding co-stimulatory domain.

SEQ ID NO: 10 sets forth the amino acid sequence of a domain found in a TRAF-binding co-stimulatory domain.

SEQ ID NO: 11 sets forth the amino acid sequence of a domain found in a TRAF-binding co-stimulatory domain.

SEQ ID NO: 12 sets forth the amino acid sequence of a Novel 6 (N6) co-stimulatory domain.

SEQ ID NO: 13 sets forth the amino acid sequence of a 4-1BB co-stimulatory domain.

SEQ ID NO: 14 sets forth the amino acid sequence of a CD8 alpha hinge domain.

SEQ ID NO: 15 sets forth the amino acid sequence of a CD8 transmembrane domain.

SEQ ID NO: 16 sets forth the amino acid sequence of a CD3 zeta intracellular signaling domain.

SEQ ID NO: 17 sets forth the amino acid sequence of a signal peptide.

SEQ ID NO: 18 sets forth the nucleic acid sequence of a JeT promoter.

SEQ ID NO: 19 sets forth the amino acid sequence of the TRC 1-2L.1592 meganuclease.

SEQ ID NO: 20 sets forth the amino acid sequence of the wild-type I-CreI meganuclease.

SEQ ID NO: 21 sets forth the amino acid sequence of a LAGLIDADG domain.

SEQ ID NO: 22 sets forth the nucleic acid sequence of a human T cell receptor alpha constant region gene.

SEQ ID NO: 23 sets forth the amino acid sequence of a polypeptide encoded by a human T cell receptor alpha constant region gene.

### DETAILED DESCRIPTION OF THE INVENTION

### 1. References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “lymphodepletion” or “lymphodepletion regimen” refers to the administration to a subject of one or more agents (e.g., chemotherapeutic lymphodepletion agents or biological lymphodepletion agents) capable of reducing endogenous lymphocytes in the subject for immunotherapy; e.g., a reduction of one or more lymphocytes (e.g., B cells, T cells, and/or NK cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment, relative to a pre determined threshold, or relative to an untreated subject).

As used herein, the term “biological lymphodepletion agent” refers to a biological material, such an antibody, antibody fragment, antibody conjugate, or the like, that can be administered as part of a lymphodepletion regimen to reduce endogenous lymphocytes in the subject for immunotherapy. In some cases, such biological lymphodepletion agents can have specificity for antigens present on lymphocytes; e.g., CD52 or CD3.

As used herein, the term “chemotherapeutic lymphodepletion agents” refers to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative.

The terms “effective dose”, “effective amount”, “therapeutically effective dose”, or “therapeutically effective amount,” as used herein, refers to an amount sufficient to effect beneficial or desirable biological and/or clinical results. In some embodiments, the effective dose is equivalent to the suggested, recommended or allowed dose (for adults or children) provided in the drug product labeling for a biological lymphodepletion agent.

The term “minimal effective dose,” as used herein, refers to weekly administration of an amount of a pharmaceutical agent that is equivalent to 10% of the maximum weekly dose as set forth by the United States Food and Drug Administration (FDA) or the European Medicines Agency (EMA), for instance, in the labeling for any drug product(s) that comprise the agent. Thus, for a biological lymphodepletion agent, a “minimal effective dose” can be 10% of the maximum dose of the agent approved for use in lymphodepletion by the FDA or the EMA.

As used herein, the term “same number of CAR T cells/kg” means the same number+/−10%. As used herein, the term “same dose of CAR T cells/kg” means the same dose (i.e., number of CAR T cells administered per kilogram)+/−10%.

As used herein, the terms “treatment”, “treating”, or “treating a subject” refers to the administration of a pharmaceutical composition disclosed herein, comprising a population of human T cells (e.g., CAR T cells), to a subject having a disease, disorder or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, a partial or complete reduction in the number of cancer cells present in the subject, and remission or improved prognosis. In some aspects, treatment includes the administration of a lymphodepletion regimen to reduce endogenous lymphocytes in the subject for immunotherapy.

As used herein, a “human T cell” or “isolated human T cell” refers to a T cell isolated from a human donor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization.

As used herein, the term “antibody” refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

As used herein, a “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain (or moiety), a transmembrane domain, and an intracellular domain (or moiety) that comprises one or more intracellular signaling domains and/or co-stimulatory domains.

In some examples, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the T cell following antigen binding. Such cytoplasmic signaling domains can include, without limitation, a CD3 zeta signaling domain (e.g., and without limitation, SEQ ID NO: 16).

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 9-11. Such intracellular co-stimulatory domains can be any of those known in the art and can include, without limitation, those co-stimulatory domains disclosed in WO 2018/067697, which is incorporated by reference herein, including, for example, Novel 6 (“N6”; SEQ ID NO: 12). Further examples of co-stimulatory domains can include 4-1 BB (CD137: SEQ ID NO: 13), CD27, CD28, CD8, OX40, CD30, CD40, PD-1, ICOS, lymphocyte function-associated antigen 1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T cell receptor (e.g., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain (SEQ ID NO: 15). Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRIIIa receptor or IgG1. In certain examples, the hinge region can be a CD8 alpha domain (SEQ ID NO: 14).

As used herein, a “chimeric antigen receptor T cell” or “CAR T cell” refers to a human T cell modified to comprise a transgene encoding a CAR, wherein the CAR is expressed on the cell surface of the T cell. The CAR T cell may be derived from any “human T cell,” as that term is used herein. For instance, a CAR T cell may be derived from a human T cell that has been isolated from a human donor, e.g., a human donor that is not the subject treated according to the method (i.e., the CAR T cells are allogeneic), but instead a healthy human donor. CAR T cells, and cells derived therefrom, may be derived from, for example, isolated T cells that have not been passaged in culture, or T cells that have been passaged and maintained under cell culture conditions without immortalization.

As used herein, the terms “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof (i.e., naturally-occurring variants), identified by NCBI Gene ID NO. 28755. See also SEQ ID NO: 22.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “CD4+” or “CD4-positive” refers to T cells, and particularly CAR T cells, that express the surface protein CD4. Such T cells can, for example, be T helper cells. As used herein, the term “CD8+” or “CD8-positive” refers to T cells that express the surface protein CD8. Such T cells can, for example, be cytotoxic cells.

As used herein, the term “CCR7+” or “CCR7-positive” refers to T cells, and particularly CAR T cells, that express the chemokine receptor CCR7 on their cell surface. CAR T cells which are both CD4+/CCR7+, or both CD8+/CCR7+, can represent CAR T cell populations having a naïve/stem cell memory phenotype, which can be characterized as CD62L+/CD45RA+/CCR7+, and/or a central memory phenotype, which can be characterized as CD62L−/CD45RO+/CCR7+.

As used herein, the term “cancer” should be understood to encompass any neoplastic disease (whether invasive or metastatic) which is characterized by abnormal and uncontrolled cell division causing malignant growth or tumor. This term embraces hematological malignancies such as B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, and pancreatic cancer.

As used herein, “acute lymphoblastic leukemia” or “ALL” refers to a cancer of the lymphoid line of blood cells characterized by the development of large numbers of immature lymphocytes.

As used herein, “non-Hodgkin lymphoma” or “NHL” refers to a group of blood cancers that includes all types of lymphoma except Hodgkin's lymphomas.

As used herein, “chronic lymphocytic leukemia” or “CLL” refers to a type of non-Hodgkin lymphoma cancer characterized by the clonal proliferation and accumulation of neoplastic B lymphocytes in the blood and bone marrow.

As used herein “small lymphocytic leukemia” or “SLL” refers to a type of non-Hodgkin lymphoma cancer characterized by the clonal proliferation and accumulation of neoplastic B lymphocytes in the lymph nodes, and spleen.

As used herein, “mantle cell lymphoma” or “MCL” refers to a type of non-Hodgkin lymphoma cancer characterized by a CD5 positive antigen-naive pre-germinal center B-cell within the mantle zone that surrounds normal germinal center follicles. MCL cells generally over-express cyclin D1 due to a chromosomal translocation in the DNA.

As used herein, “diffuse large B cell lymphoma” or “DLBCL” refers to a non-Hodgkin lymphoma affecting B cells that can develop in the lymph nodes or in extranodal sites (areas outside the lymph nodes) such as the gastrointestinal tract, testes, thyroid, skin, breast, bone, brain, or essentially any organ of the body.

As used herein, the terms “response,” “complete response,” “complete response with incomplete blood count recovery,” “refractory disease,” “partial response,” “disease progression” or “progressive disease,” “refractory disease,” “relapse” or “relapsed disease” each refer to assessments of disease state and response in subjects following treatment according to the methods disclosed herein. For example, the response criteria for the assessment of subjects with B-ALL are based on the NCCN Guidelines (NCCN, 2017). As described therein, a complete response is defined as no circulating blasts or extramedullary disease, no lymphadenopathy, splenomegaly, skin/gum infiltration/testicular mass/CNS involvement, trilineage hematopoiesis and <5% blasts, absolute neutrophil count (ANC)>1000/mm³, platelets >100,000/mm³, and no recurrence for 4 weeks. Complete response with incomplete blood count recovery (CRi) is defined as meeting all criteria for complete response except platelet count and/or ANC. An overall response rate (ORR) can be calculated as CR+CRi. Refractory disease can be defined as failure to achieve a complete response at the end of induction. Progressive disease can be defined as an increase of at least 25% in the absolute number of circulating or bone marrow blasts or development of extramedullary disease. Relapsed disease can be defined as the reappearance of blasts in the blood or bone marrow (>5%) or in any extramedullary site after a complete response.

For NHL, response criteria for local and central assessments of subjects with NHL are based on the revised Lugano Classification (Cheson et al, 2016), which incorporates PET-CT. A complete response (i.e., a complete metabolic response) is characterized by lymph nodes and extralymphatic sites having a score of 1, 2, or 3 with or without a residual mass on 5-point scale (5PS), and it is recognized that in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. A complete response is further characterized by no new lesions and no evidence of fluorodeoxyglucose (FDG)-avid disease in marrow. A partial response (i.e., partial metabolic response) is characterized by lymph nodes and extralymphatic sites having a score of 4 or 5 with reduced uptake compared with Baseline and residual mass(es) of any size. At interim, these findings suggest responding disease. At end of treatment, these findings indicate residual disease. A partial response is further characterized by no new lesions and bone marrow wherein residual uptake is higher than uptake in normal marrow but reduced compared with baseline. No response or stable disease (i.e., no metabolic response) is characterized by target nodes/nodal masses and/or extranodal lesions having a score of 4 or 5 with no significant change in FDG uptake from baseline at interim or end of treatment, no new lesions, and no change in bone marrow from baseline. Progressive disease (i.e., progressive metabolic disease) is characterized by individual target nodes/nodal masses having a score 4 or 5 with an increase in intensity of uptake from baseline and/or new foci compatible with lymphoma, new FDG-avid foci consistent with lymphoma at interim or end-of-treatment assessment, no non-measured lesions, new FDG-avid foci consistent with lymphoma rather than another etiology (e.g., infection, inflammation), and new or recurrent FDG-avid foci. RECIL 2017 criteria can also be used to asses response based on assessment of target lesions. A complete response is characterized by complete disappearance of all target lesions and all nodes with long axis <10 mm, ≥30% decrease in the sum of longest diameters of target lesions (PR) with normalization of FDG-PET, normalization of FDG-PET (Deauville score 1-3), no involvement of bone marrow, and no new lesions. A partial response is characterized by ≥30% decrease in the sum of longest diameters of target lesions but not a complete response, a positive FDG-PET (Deauville score 4-5), any bone marrow involvement, and no new lesions. A minor response is characterized by ≥10% decrease in the sum of longest diameters of target lesions but not a PR (<30%), any FDG-PET, any bone marrow involvement, and no new lesions. Stable disease is characterized by <10% decrease or ≤20% increase in the sum of longest diameters of target lesions, any FDG-PET, any bone marrow involvement, and no new lesions. Progressive disease is characterized by >20% increase in the sum of longest diameters of target lesions, for small lymph nodes measuring <15 mm post therapy, a minimum absolute increase of 5 mm and the long diameter should exceed 15 mm, appearance of a new lesion, any FDG-PET, any bone marrow involvement, and the appearance of new lesions or no new lesions.

As used herein, the term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, an “anti-CD52 antibody” refers to an antibody, or antibody fragment or conjugate, having specificity for a CD52 protein expressed on the cell surface of human T cells. In some examples, an anti-CD52 antibody can be a monoclonal antibody. In some cases, an anti-CD52 antibody can be alemtuzumab (i.e., CAMPATH). In some cases, an anti-CD52 antibody can be ALLO-647 (Allogene Therapeutics, San Francisco, Calif.).

As used herein, an “anti-CD3 antibody” refers to an antibody, or antibody fragment or conjugate, having specificity for a CD3 protein expressed on the cell surface of human T cells. In some examples, an anti-CD3 antibody can be a monoclonal antibody. In some cases, an anti-CD3 antibody can be muromonah-CD3 (Orthoclone OKT3™), otelixizumab, teplizumab, foralumab, visilizumab, or derivatives thereof which have specificity for CD3.

As used herein, “detectable cell surface expression of CD3” refers to the ability to detect CD3 on the cell surface of a cell (e.g., a genetically-modified cell described herein) using standard experimental methods. Such methods can include, for example, immunostaining and/or flow cytometry specific for CD3. In certain embodiments, the method for determining detectable cell surface expression of CD3 in the disclosed methods is flow cytometry specific for CD3. Methods for detecting cell surface expression of CD3 on a cell include those described in the examples herein, and, for example, those described in MacLeod et al. (2017) Molecular Therapy 25(4): 949-961, the entire disclosure of which is incorporated by reference herein.

As used herein, “detectable cell surface expression of an endogenous alpha/beta TCR” refers to the ability to detect one or more components of the TCR complex (e.g., an alpha/beta TCR complex) on the cell surface of a T cell (e.g., a CAR T cell), or a population of T cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. In certain embodiments, the method for determining detectable cell surface expression of an endogenous alpha/beta TCR in the disclosed methods is flow cytometry specific for CD3. Methods for detecting cell surface expression of an endogenous TCR (e.g., an alpha/beta TCR) on an immune cell include those described in MacLeod et al. (2017) Molecular Therapy 25(4): 949-961.

As used herein, the term “no detectable cell surface expression of CD3” refers to lack of detection of CD3 on the surface of a T cell (e.g., a CAR T cell) described herein, or population of T cells (e.g., CAR T cells) described herein, within the limits of detection of standard experimental methods in the art. Methods for detecting cell surface expression of CD3 on an immune cell include those described in MacLeod et al. (2017). This term may embrace, in some examples, no detectable cell surface expression of an endogenous alpha/beta TCR, using one or more standard methods for detecting cell surface expression of an endogenous TCR on an immune cell, as CD3 is a component of the assembled cell surface TCR complex.

As used herein, the term “proliferate in vivo” refers to an expansion in the number of T cells, such as CAR T cells described herein, in a subject following administration during immunotherapy. Such proliferation or expansion can be determined by methods known in the art and those shown in the examples herein, which include, for example, utilizing PCR analysis to determine the number of copies of a CAR transgene per μg of DNA isolated from peripheral blood mononuclear cells over a time course following administration of the pharmaceutical composition comprising CAR T cells, or using flow cytometry to determine the number of CAR-positive T cells in blood.

As used herein, the terms “nuclease” and “endonuclease” are used interchangeably to refer to naturally-occurring or engineered enzymes which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. A meganuclease can be an endonuclease that is derived from I-CreI (SEQ ID NO: 20), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in cells, particularly in human immune cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S 1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type Its restriction endonuclease, including but not limited to FokI, FoM, StsI, HhaI, HindIII, Nod, BbvCI, EcoRI, BglI, and AlwI. Additional Type Its restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12^{th} and 13^{th} amino acids. These two positions, referred to as the repeat variable dipeptide (RVD), are highly variable and show a strong correlation with specific nucleotide recognition. Each base pair in the DNA target sequence is contacted by a single TAL repeat, with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein which binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, EndI, I-Bast, I-TevII, I-Tevlll, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the term “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S 1 nuclease, mung bean nuclease, pancreatic DNAse I, micrococcal nuclease, and yeast HO endonuclease. Nuclease domains useful for the design of zinc finger nucleases include those from a Type Its restriction endonuclease, including but not limited to Fold, FoM, and StsI restriction enzyme. Additional Type Its restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) which bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a Cpf1 CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease. This term embraces chromosomal DNA duplexes as well as single-stranded chromosomal DNA.

As used herein, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, a “template nucleic acid” or “donor template” refers to a nucleic acid sequence that is desired to be inserted into a cleavage site within a cell's genome. Such template nucleic acids or donor templates can comprise, for example, a transgene, such as an exogenous transgene, which encodes a protein of interest (e.g., a CAR). The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, with respect to a protein, the term “recombinant” or “engineered” means having an altered amino acid sequence as a result of the application of genetic engineering techniques to nucleic acids which encode the protein, and cells or organisms which express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein but produced by cloning and expression in a heterologous host, is not considered recombinant.

As used herein, the term “exogenous” or “heterologous” in reference to a nucleotide sequence or amino acid sequence is intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention.

As used herein, a “vector” can also refer to a viral vector. Viral vectors can include, without limitation, retroviral vectors, lentiviral vectors, adenoviral vectors, and adeno-associated viral vectors (AAV).

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and 5-2 if the variable is inherently continuous.

### 2. Lymphodepletion Regimens

The present invention includes methods of immunotherapy for treating cancer in a subject in need thereof. Such methods include administering to the subject a lymphodepletion regimen, for example, prior to administration of a pharmaceutical composition comprising a population of human T cells, including CAR T cells.

In some embodiments, the lymphodepletion regimen includes no more than a minimal effective dose, as defined herein, of any biological lymphodepletion agent In some examples, the lymphodepletion regimen does not include administration of any biological lymphodepletion agent in an amount greater than about 0.01 mg/kg, 0.03 mg/kg, 0.05 mg/kg, 0.75 mg/kg, 0.1 mg/kg, 0.15 mg/kg, 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg during the 7 day period preceding administration of a pharmaceutical composition of the invention (e.g., comprising a population of T cells, including CAR T cells). Thus, in certain examples, the lymphodepletion regimen does not include administration of any biological lymphodepletion agent in an amount greater than about 0.01 mg/kg during the preceding 7 day period. In certain examples, the lymphodepletion regimen does not include administration of any biological lymphodepletion agent in an amount greater than about 0.03 mg/kg during the preceding 7 day period. In certain examples, the lymphodepletion regimen does not include administration of any biological lymphodepletion agent in an amount greater than about 0.05 mg/kg during the preceding 7 day period. In certain examples, the lymphodepletion regimen does not include administration of any biological lymphodepletion agent in an amount greater than about 0.075 mg/kg during the preceding 7 day period. In certain examples, the lymphodepletion regimen does not include administration of any biological lymphodepletion agent in an amount greater than about 0.1 mg/kg during the preceding 7 day period. In certain examples, the lymphodepletion regimen does not include administration of any biological lymphodepletion agent in an amount greater than about 0.15 mg/kg during the preceding 7 day period. In certain examples, the lymphodepletion regimen does not include administration of any biological lymphodepletion agent in an amount greater than about 0.2 mg/kg during the preceding 7 day period. In certain examples, the lymphodepletion regimen does not include administration of any biological lymphodepletion agent in an amount greater than about 0.3 mg/kg during the preceding 7 day period. In certain examples, the lymphodepletion regimen does not include administration of any biological lymphodepletion agent in an amount greater than about 0.4 mg/kg during the preceding 7 day period. In certain examples, the lymphodepletion regimen does not include administration of any biological lymphodepletion agent in an amount greater than about 0.5 mg/kg during the preceding 7 day period. In certain examples, the lymphodepletion regimen does not include administration of any biological lymphodepletion agent in an amount greater than about 0.6 mg/kg during the preceding 7 day period. In certain examples, the lymphodepletion regimen does not include administration of any biological lymphodepletion agent in an amount greater than about 0.7 mg/kg during the preceding 7 day period. In certain examples, the lymphodepletion regimen does not include administration of any biological lymphodepletion agent in an amount greater than about 0.8 mg/kg during the preceding 7 day period. In certain examples, the lymphodepletion regimen does not include administration of any biological lymphodepletion agent in an amount greater than about 0.9 mg/kg during the preceding 7 day period. In certain examples, the lymphodepletion regimen does not include administration of any biological lymphodepletion agent in an amount greater than about 1.0 mg/kg during the preceding 7 day period.

A biological lymphodepletion agent can be, for example, any biological material, such an antibody, antibody fragment, antibody conjugate, or the like, that can be administered as part of a lymphodepletion regimen to reduce endogenous lymphocytes in the subject for immunotherapy. Such biological lymphodepletion agents can include, for example, a monoclonal antibody, or a fragment thereof. In some examples, the biological lymphodepletion agent has specificity for a T cell antigen; i.e., an antigen expressed on the cell surface of T cells. Examples of such antigens include, without limitation, CD52 and CD3. In a particular example, the biological lymphodepletion agent is an antibody, such as a monoclonal antibody, having specificity for CD52. Such antibodies can include, for example, alemtuzumab (i.e., CAMPATH), ALLO-647 (Allogene Therapeutics, San Francisco, Calif.), derivatives thereof which bind CD52, or any other CD52 antibody. In another particular example, the biological lymphodepletion agent is an antibody, such as a monoclonal antibody, having specificity for CD3. In some cases, an anti-CD3 antibody can be muromonab-CD3 (Orthoclone OKT3™), otelixizumab, teplizumab, foralumab, visilizumab, or derivatives thereof which have specificity for CD3.

Lymphodepletion regimens of the invention include the administration of one or more chemotherapeutic lymphodepletion agents. Pre-treatment or pre-conditioning patients prior to cell therapies with one or more chemotherapeutic lymphodepletion agents improves the efficacy of the cellular therapy by reducing the number of endogenous host lymphocytes in the subject, thereby providing a more optimal environment for administered cells to proliferate once administered to the subject. An effective dose of one or more chemotherapeutic lymphodepletion agents can result in the reduction of one or more endogenous lymphocytes (e.g., B cells, T cells, and/or NK cells) in the subject by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control; e.g., relative to a starting amount in the subject undergoing treatment, relative to a pre-determined threshold, or relative to an untreated subject.

In some embodiments, 1, 2, 3, 4, or more chemotherapeutic lymphodepletion agents may be included in the lymphodepletion regimen.

Chemotherapeutic lymphodepletion agents can refer to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative. Chemotherapeutic lymphodepletion agents can include those known in the art include, without limitation, cyclophosphamide, fludarabine, bendamustine, melphalan, 6-mercaptopurine (6-MP), daunorubicin, cytarabine, L-asparaginase, methotrexate, prednisone, dexamethasone, nelarabine, or combinations thereof. In some embodiments, the chemotherapeutic lymphodepletion agent is fludarabine. In some embodiments, the chemotherapeutic lymphodepletion agent is cyclophosphamide. In certain embodiments, the methods herein involve administering a combination of chemotherapeutic lymphodepletion agents, such as a combination of fludarabine and cyclophosphamide.

The lymphodepletion regimen administered during the method of the invention can be administered in an amount effective (i.e., an effective dose) to deplete or reduce the quantity of endogenous lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control, e.g., relative to a starting amount in the subject undergoing treatment, relative to a pre determined threshold, or relative to an untreated subject, prior to administration of the pharmaceutical composition (e.g., a population of human T cells, including CAR T cells). The reduction in lymphocyte count can be monitored using conventional techniques known in the art, such as by flow cytometry analysis of cells expressing characteristic lymphocyte cell surface antigens in a blood sample withdrawn from the subject at varying intervals during treatment with the antibody. According to some embodiments, when the concentration of lymphocytes has reached a minimum value in response to the lymphodepletion regimen, the physician may conclude the lymphodepletion therapy and may begin preparing the subject for administration of the pharmaceutical composition.

In various embodiments, the one or more chemotherapeutic lymphodepletion agents can be administered one day to one month (e.g., 1 day, 2 days, 3 days, 4 days, 5 days, 6 days, 7 days, 8 days, 9 days, 10 days, 11 days, 12 days, 13 days. 14 days, 15 days, 16 days, 17 days. 18 days, 19 days, 20 days, 21 days, 22 days. 23 days, 24 days, 25 days, 26 days. 27 days, 28 days, 29 days, or 30 days) prior to administration of the pharmaceutical compositions described herein. In some embodiments, a chemotherapeutic lymphodepletion agent is administered to the subject three or more days prior to administration of the pharmaceutical composition. In certain embodiments, administration of a chemotherapeutic lymphodepletion agent ends at least one day, at least two days, or at least three days prior to administration of the pharmaceutical composition.

In some embodiments, a chemotherapeutic lymphodepletion agent can be administered as a single dose per day on each of eight consecutive days, as a single dose per day on each of seven consecutive days, as a single dose per day on each of six consecutive days, as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition.

In some embodiments, the chemotherapeutic lymphodepletion agent is cyclophosphamide, which is administered as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition. In certain embodiments, the cyclophosphamide is administered as one dose per day for three consecutive days or one dose per day for two consecutive days. In certain embodiments, administration of cyclophosphamide ends at least one to three days prior to administration of the pharmaceutical composition. In certain embodiments, cyclophosphamide is administered as a single dose on each day beginning five days and ending three days before administration of the pharmaceutical composition.

In some embodiments, the chemotherapeutic lymphodepletion agent is fludarabine, which is administered as a single dose per day on each of five consecutive days, as a single dose per day on each of four consecutive days, as a single dose per day on each of three consecutive days, as a single dose per day on each of two consecutive days, or as a single dose on one day, prior to administration of the pharmaceutical composition. In other embodiments, the fludarabine is administered as one dose per day for five consecutive days or as one dose per day for three consecutive days. In certain embodiments, administration of fludarabine ends at least one to three days prior to administration of the genetically-modified cells. In certain embodiments, fludarabine is administered as a single dose on each day beginning five days and ending three days before administration of the pharmaceutical composition. In certain embodiments, fludarabine is administered as a single dose on each beginning six days and ending three days before administration of the pharmaceutical composition.

In some embodiments, cyclophosphamide is administered to the subject daily starting five days and ending three days prior to administration of the pharmaceutical composition. In some embodiments, cyclophosphamide is administered to the subject daily starting five days and ending two days prior to administration of the pharmaceutical composition. In some embodiments, cyclophosphamide is administered to the subject daily starting four days and ending three days prior to administration of the pharmaceutical composition. In some embodiments, cyclophosphamide is administered to the subject daily starting four days and ending two days prior to administration of the pharmaceutical composition.

In particular embodiments, fludarabine is administered to the subject daily starting five days and ending three days prior to administration of the pharmaceutical composition. In some embodiments, fludarabine is administered to the subject daily starting five days and ending two days prior to administration of the pharmaceutical composition. In other particular embodiments, fludarabine is administered to the subject daily starting seven days and ending three days prior to administration of the pharmaceutical composition. In other particular embodiments, fludarabine is administered to the subject daily starting seven days and ending two days prior to administration of the pharmaceutical composition. In other particular embodiments, fludarabine is administered to the subject daily starting six days and ending three days prior to administration of the pharmaceutical composition.

In certain embodiments, cyclophosphamide is administered to the subject daily starting five days and ending three days prior to administration of the pharmaceutical composition, and fludarabine is administered to the subject daily starting five days and ending three days prior to administration of the pharmaceutical composition. In some embodiments, cyclophosphamide is administered to the subject daily starting five days and ending two days prior to administration of the pharmaceutical composition, and fludarabine is administered to the subject daily starting five days and ending two days prior to administration of the pharmaceutical composition. In other embodiments, cyclophosphamide is administered to the subject daily starting four days and ending three days prior to administration of the pharmaceutical composition, and fludarabine is administered to the subject at a dose daily starting seven days and ending three days prior to administration of the pharmaceutical composition. In other embodiments, cyclophosphamide is administered to the subject daily starting four days and ending two days prior to administration of the pharmaceutical composition, and fludarabine is administered to the subject at a dose daily starting seven days and ending two days prior to administration of the pharmaceutical composition. In certain embodiments, cyclophosphamide is administered to the subject daily starting five days and ending three days prior to administration of the pharmaceutical composition, and fludarabine is administered to the subject daily starting six days and ending three days prior to administration of the pharmaceutical composition.

For example, in embodiments where the chemotherapeutic lymphodepletion agent is cyclophosphamide, and the dose can be adjusted based on the desired effect. In some embodiments, the dose of cyclophosphamide can be higher than about 400 mg/m²/day and lower than about 1500 mg/m²/day. In some embodiments, the dose of cyclophosphamide is about 400-1500 mg/m²/day, about 400-1500 mg/m²/day, about 400-1500 mg/m²/day, about 450-1500 mg/m²/day, about 500-1500 mg/m²/day, about 550-1500 mg/m²/day, or about 600-1500 mg/m²/day. In another embodiment, the dose of cyclophosphamide is about 400-1500 mg/m²/day, about 400-1000 mg/m²/day, about 400-900 mg/m²/day, about 450-800 mg/m²/day, about 450-700 mg/m²/day, about 450-600 mg/m²/day, or about 450-550 mg/m²/day. In certain embodiments, the dose of cyclophosphamide is about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, about 1000 mg/m²/day, about 1100 mg/m²/day, about 1200 mg/m²/day, about 1300 mg/m²/day, about 1400 mg/m²/day, or about 1500 mg/m²/day. In one particular embodiment, the dose of cyclophosphamide is about 500 mg/m²/day. In one particular embodiment, the dose of cyclophosphamide is about 1000 mg/m²/day.

In the present invention, the dose of fludarabine can also be adjusted depending on the desired effect. For example, the dose of fludarabine can be higher than mg/m²/day and lower than mg/m²/day. In some embodiments, the dose of fludarabine is about 25-100 mg/m²/day, about 30-100 mg/m²/day, about 35-100 mg/m²/day, about 40-100 mg/m²/day, about 45-100 mg/m²/day, about 50-100 mg/m²/day, about 55-100 mg/m²/day, or about 60-100 mg/m²/day. In other embodiments, the dose of fludarabine is about 25-100 mg/m²/day, about 25-90 mg/m²/day, about 25-80 mg/m²/day, about 25-70 mg/m²/day, about 25-60 mg/m²/day, about 25-50 mg/m²/day, about 25-45 mg/m²/day, about 25-40 mg/m²/day, about 25-35 mg/m²/day, or about 28-32 mg/m²/day. In certain embodiments, the dose of fludarabine is about 25 mg/m²/day, 30 mg/m²/day, 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In one particular embodiment, the dose of fludarabine is about 30 mg/m²/day.

In some embodiments, the dose of cyclophosphamide is about 400-1500 mg/m²/day and the dose of fludarabine is about 25-100 mg/m²/day. In certain embodiments, the dose of cyclophosphamide is about 500 mg/m²/day and the dose of fludarabine is about 30 mg/m²/day. In other particular embodiments, the dose of cyclophosphamide is about 1000 mg/m²/day and the dose of fludarabine is about 30 mg/m²/day. In other particular embodiments, the dose of cyclophosphamide is between about 500-1500 mg/m²/day and the dose of fludarabine is about 30 mg/m²/day.

In particular embodiments, cyclophosphamide is administered to the subject at a dose of about 500 mg/m²/day daily starting five days and ending three days prior to administration of the pharmaceutical composition. In particular embodiments, cyclophosphamide is administered to the subject at a dose of about 500 mg/m²/day daily starting five days and ending two days prior to administration of the pharmaceutical composition. In other particular embodiments, cyclophosphamide is administered to the subject at a dose of about 500-1500 mg/m²/day daily starting four days and ending three days prior to administration of the pharmaceutical composition. In other particular embodiments, cyclophosphamide is administered to the subject at a dose of about 500-1500 mg/m²/day daily starting four days and ending two days prior to administration of the pharmaceutical composition. In certain embodiments, cyclophosphamide is administered to the subject at a dose of about 1000 mg/m²/day daily starting five days and ending three days prior to administration of the pharmaceutical composition.

In particular embodiments, fludarabine is administered to the subject at a dose of about 30 mg/m²/day daily starting five days and ending three days prior to administration of the pharmaceutical composition. In particular embodiments, fludarabine is administered to the subject at a dose of about 30 mg/m²/day daily starting five days and ending two days prior to administration of the pharmaceutical composition. In other particular embodiments, fludarabine is administered to the subject at a dose of about 30 mg/m²/day daily starting seven days and ending three days prior to administration of the pharmaceutical composition. In other particular embodiments, fludarabine is administered to the subject at a dose of about 30 mg/m²/day daily starting seven days and ending two days prior to administration of the pharmaceutical composition. In certain embodiments, fludarabine is administered to the subject at a dose of about 30 mg/m²/day daily starting six days and ending three days prior to administration of the pharmaceutical composition.

In certain embodiments, cyclophosphamide is administered to the subject at a dose of about 500 mg/m²/day daily starting five days and ending three days prior to administration of the pharmaceutical composition, and fludarabine is administered to the subject at a dose of about 30 mg/m²/day daily starting five days and ending three days prior to administration of the pharmaceutical composition. In certain embodiments, cyclophosphamide is administered to the subject at a dose of about 500 mg/m²/day daily starting five days and ending two days prior to administration of the pharmaceutical composition, and fludarabine is administered to the subject at a dose of about 30 mg/m²/day daily starting five days and ending two days prior to administration of the pharmaceutical composition.

In yet further embodiments, cyclophosphamide is administered to the subject at a dose of about 500-1500 mg/m²/day daily starting four days and ending three days prior to administration of the pharmaceutical composition, and fludarabine is administered to the subject at a dose of about 30 mg/m²/day daily starting seven days and ending three days prior to administration of the pharmaceutical composition. In yet further embodiments, cyclophosphamide is administered to the subject at a dose of about 500-1500 mg/m²/day daily starting four days and ending two days prior to administration of the pharmaceutical composition, and fludarabine is administered to the subject at a dose of about 30 mg/m²/day daily starting seven days and ending two days prior to administration of the pharmaceutical composition.

In yet further embodiments, cyclophosphamide is administered to the subject at a dose of about 1000 mg/m²/day daily starting four days and ending three days prior to administration of the pharmaceutical composition, and fludarabine is administered to the subject at a dose of about 30 mg/m²/day daily starting seven days and ending three days prior to administration of the pharmaceutical composition. In yet further embodiments, cyclophosphamide is administered to the subject at a dose of about 1000 mg/m²/day daily starting four days and ending two days prior to administration of the pharmaceutical composition, and fludarabine is administered to the subject at a dose of about 30 mg/m²/day daily starting seven days and ending two days prior to administration of the pharmaceutical composition. In certain embodiments, cyclophosphamide is administered to the subject at a dose of about 1000 mg/m²/day daily starting five days and ending three days prior to administration of the pharmaceutical composition, and fludarabine is administered to the subject at a dose of about 30 mg/m²/day daily starting six days and ending three days prior to administration of the pharmaceutical composition.

### 3. Human T Cells and Populations of CAR T Cells

The invention provides pharmaceutical compositions comprising populations of human T cells, wherein a plurality of the human T cells are CAR T cells; i.e., T cells comprising in their genome a transgene encoding a CAR, wherein the CAR is expressed on the cell surface of the T cell.

T cells for use in the invention can be obtained from a number of sources including, for example, peripheral blood mononuclear cells, bone marrow, lymph node tissue, cord blood, thymus tissue, tissue from a site of infection, ascites, pleural effusion, spleen tissue, and tumors. In some embodiments of the present disclosure, immune cells (e.g., T cells) are obtained from a unit of blood collected from a subject using any number of techniques known to the skilled artisan. In one embodiment, cells from the circulating blood of an individual are obtained by apheresis.

Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

In some embodiments, a CAR useful in the invention comprises an extracellular ligand-binding domain having specificity for a cancer cell antigen (i.e., an antigen expressed on the surface of a cancer cell). The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR can include those associated cancer cells. In some embodiments, a CAR is engineered to target a cancer-specific antigen of interest by way of engineering a desired ligand-binding moiety that specifically binds to an antigen on a cancer cell. In the context of the present disclosure, “cancer antigen” or “cancer-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. As non-limiting examples, in some embodiments the antigen of the target is CD19, CD20, or B cell maturation antigen (BCMA; i.e., CD269).

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

In certain embodiments, the ligand-binding domain of the CAR is an scFv. In some such embodiments, the scFv comprises a heavy chain variable (VH) domain and a light chain variable (VL) domain from a monoclonal antibody having specificity for a cancer cell antigen. In some examples, the scFv comprises a VH domain and a VL domain obtained from a CD19-specific antibody. In certain examples, the VH domain comprises SEQ ID NO: 3 and the VL domain comprises SEQ ID NO: 4. In some such examples, the CAR can comprise an amino acid sequence having at least 80%, at least 85%, at least 90%, at least 95%, or more, sequence identity to SEQ ID NO: 5, wherein the CAR has specificity for CD19. In some embodiments, the CAR comprises SEQ ID NO: 5. In some embodiments, the CAR comprises an amino acid sequence that differs from the sequence of SEQ ID NO: 5 by 1, 2, 3, 4, 5, 6, 7, 8, 9, 10 or more than 10 amino acids.

In some examples, the scFv comprises a VH domain and a VL domain obtained from a CD20-specific antibody. In certain examples, the VH domain comprises SEQ ID NO: 6 and the VL domain comprises SEQ ID NO: 7. In some such examples, the CAR can comprise an amino acid sequence having at least 80%, at least 85%, at least 90%, at least 95%, or more, sequence identity to SEQ ID NO: 8, wherein the CAR has specificity for CD19. In some embodiments, the CAR comprises SEQ ID NO: 8. In some embodiments, the CAR comprises an amino acid sequence that differs from the sequence of SEQ ID NO: 8 by 1, 2, 3, 4, 5, 6, 7, 8, 9, 10 or more than 10 amino acids.

In some examples, the scFv comprises a VH domain and a VL domain obtained from a BCMA-specific antibody.

In some embodiments, a CAR comprises a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex). IL2 receptor p55 (α chain), p75 (β chain) or γ chain, subunit chain of Fc receptors (e.g., Fcy receptor III) or CD proteins such as the CD8 alpha chain. In certain examples, the transmembrane domain is a CD8 alpha domain (SEQ ID NO: 15). Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRIIIa receptor or IgG1. In certain examples, the hinge region can be a CD8 alpha domain (SEQ ID NO: 14).

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain (SEQ ID NO: 16).

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 9-11. Such intracellular co-stimulatory domains can be any of those known in the art and can include, without limitation, those co-stimulatory domains disclosed in WO 2018/067697 including, for example, Novel 6 (“N6”; SEQ ID NO: 12). Further examples of co-stimulatory domains can include 4-1BB (CD137; SEQ ID NO: 13), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

The CARs described herein have specificity for cancer cell antigens. Such cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

CAR T cells of the present invention comprise an inactivated TCR alpha gene and/or an inactivated TCR beta gene. Inactivation of the TCR alpha gene and/or TCR beta gene to generate the CAR T cells of the present invention occurs in at least one or both alleles where the TCR alpha gene and/or TCR beta gene is being expressed. Thus, inactivation may occur by disruption of one of the alleles of the TCR alpha or TCR beta gene. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in CAR T cells that have no detectable cell surface expression of the endogenous alpha/beta TCR. The endogenous alpha/beta TCR incorporates CD3. Therefore, cells with an inactivated TCR alpha gene and/or TCR beta chain can have no detectable cell surface expression of CD3, e.g., as determined by flow cytometry specific for CD3. In particular embodiments, the inactivated gene is a TCR alpha constant region (TRAC) of the TCR alpha gene.

In some examples, the TCR alpha gene, the TRAC region, or the TCR beta gene is inactivated by insertion of a transgene encoding the CAR. In some examples, one or both alleles of the TCR alpha gene is inactivated by insertion of a transgene encoding the CAR. In some embodiments, one or both alleles of the TRAC region is inactivated by insertion of a transgene encoding the CAR. In some examples, one or both alleles of the TCR beta gene is inactivated by insertion of a transgene encoding the CAR. Insertion of the CAR transgene disrupts expression of the endogenous TCR alpha chain or TCR beta chain and, therefore, prevents assembly of an endogenous alpha/beta TCR on the T cell surface. In some examples, the CAR transgene is inserted into the TRAC gene. In a particular example, a CAR transgene is inserted into the TRAC gene at an engineered meganuclease recognition sequence comprising SEQ ID NO: 1. In particular examples, the CAR transgene is inserted into SEQ ID NO: 1 between nucleotide positions 13 and 14.

As used herein, “detectable cell surface expression of an endogenous alpha/beta TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in the examples herein, and, for example, those described in MacLeod et al. (2017).

Similarly, “detectable cell surface expression of CD3” refers to lack of detection of CD3 on the surface of a T cell (e.g., a CAR T cell) described herein, or population of T cells (e.g., CAR T cells) described herein, as detected using standard experimental methods in the art. Methods for detecting cell surface expression of CD3 on an immune cell include those described in MacLeod et al. (2017).

Human T cells modified by the present invention may require activation prior to introduction of a nuclease and/or an exogenous sequence of interest to generate CAR T cells. For example, T cells can be contacted with anti-CD3 and anti-CD28 antibodies that are soluble or conjugated to a support (e.g., beads) for a period of time sufficient to activate the cells.

CAR T cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

In various embodiments of the invention, the pharmaceutical composition comprises a population of human T cells. This population of human T cells includes a plurality of CAR T cells expressing a cell surface CAR. In some examples, the CAR T cells represent between about 50% and 80% of the human T cells in the population. In some examples, the CAR T cells represent between about 40% and 75% of the human T cells in the population. In some examples, the CAR T cells represent between about 50% and 70% of the human T cells in the population. In some examples, the CAR T cells represent between about 55% and 70% of the human T cells in the population. In some examples, the CAR T cells represent between about 58% and 69% of the human T cells in the population.

In some examples of the population of human T cells, no more than about 0.5% of the cells in the population have detectable cell surface expression of CD3. In certain examples, no more than about 0.3% of the cells in the population have detectable cell surface expression of CD3. In certain examples, no more than about 0.2% of the cells in the population have detectable cell surface expression of CD3. In certain examples, no more than about 0.1% of the cells in the population have detectable cell surface expression of CD3. In certain examples, no cells in the population have detectable cell surface expression of CD3.

In some embodiments, the ratio of CD4+ CAR T cells to CD8+ CAR T cells in the population is between about 0.3 and about 3.0. In certain embodiments, the ratio of CD4+ CAR T cells to CD8+ CAR T cells in the population is between about 0.7 and about 2.5.

CAR T cells which are both CD4+/CCR7+, or both CD8+/CCR7+, can represent CAR T cell populations having a naïve/stem cell memory phenotype, which can be characterized as CD62L+/CD45RA+/CCR7+, and/or a central memory phenotype, which can be characterized as CD62L−/CD45RO+/CCR7+. In some embodiments, the percentage of CD4+ CAR T cells in the population that are also CCR7+ is between about 35% to about 75%. In certain embodiments, the percentage of CD4+ CAR T cells in the population that are also CCR7+ is between about 35% to about 70%. In certain embodiments, the percentage of CD4+ CAR T cells in the population that are also CCR7+ is between about 40% to about 68%.

In some embodiments, the percentage of CD8+ CAR T cells in the population that are also CCR7+ is between about 25% and about 50%. In some embodiments, the percentage of CD8+ CAR T cells in the population that are also CCR7+ is between about 25% and about 45%. In certain embodiments, the percentage of CD8+ CAR T cells in the population that are also CCR7+ is between about 31% and about 42%.

### 4. Methods for Genetically-Modifying T Cells

The present invention provides populations of human T cells comprising a plurality of CAR T cells that have been genetically-modified to inactivate a TCR alpha gene and/or a TCR beta gene. In particular examples, the inactivated gene can be a TCR alpha constant region (TRAC) gene. Such gene inactivations can disrupt expression of the endogenous TCR alpha chain and/or the endogenous TCR beta chain, which are each necessary for the assembly of the endogenous alpha/beta TCR. Thus, inactivation of one or more of these genes results in CAR T cells that do not have detectable cell surface express of an endogenous alpha/beta TCR and, consequently, do not have detectable cell surface expression of CD3 which is part of the TCR complex.

In some examples, inactivation of the TCR alpha gene, TCR beta gene, and/or the TRAC region can result from the insertion of a transgene into one or both alleles of any one of these endogenous genes. Insertion of the transgene disrupts expression of the polypeptide encoded by the gene; e.g., the endogenous TCR alpha chain or the endogenous TCR beta chain. In some examples, the transgene encodes the CAR which is expressed by the cell and localized to the cell surface.

In some examples, CAR T cells utilized in the methods can be made, for example, by a manufacturing process comprising the following steps: (a) a first culturing step wherein isolated human T cells are cultured in media for 3 days with anti-CD3 and anti-CD28 antibodies bound to a matrix or particle; (b) electroporating the isolated human T cells to introduce mRNA encoding an engineered nuclease having specificity for a recognition sequence within a TCR alpha gene, a TRAC gene, or a TCR beta gene, wherein the engineered nuclease is expressed in the human T cells and generates a cleavage site at the recognition sequence; (c) transducing the isolated human T cells with a recombinant AAV vector comprising a donor template, wherein the donor template comprises a transgene encoding the CAR, and wherein the donor template is flanked by a 5′ homology arm having homology to sequences 5′ upstream of the cleavage site, and by a 3′ homology arm having homology to sequences 3′ downstream of the cleavage site, wherein the donor template is inserted into the genome of the isolated human T cells at the cleavage site; (d) a second culturing step wherein the isolated human T cells are cultured in media for about 5 days; (e) removing the isolated human T cells that express cell surface CD3 using anti-CD3 antibodies; and (f) a third culturing step wherein the isolated human T cells are cultured in media to expand the number of cells and to generate the population of human T cells comprising a plurality of CAR T cells.

In some embodiments, the method can comprise a further step of concentrating the population of human T cells after the third culturing step. The method can further comprise an additional step of formulating the population of human T cells in cryopreservation media after the concentrating. In some embodiments, the manufacturing is completed in about 10 days or less. In certain embodiments, the anti-CD3 and anti-CD28 antibodies are bound to beads. In some embodiments, the anti-CD3 antibodies are conjugated to magnetic beads. In some embodiments, the recombinant AAV vector has a serotype of AAV6.

Insertion of the donor template comprising the CAR transgene can be achieved by use of an engineered nuclease to generate a cleavage site within a recognition sequence in the genome, such as within the TCR alpha gene, the TRAC gene, or the TCR beta gene.

Any engineered nuclease can be used for targeted insertion of the donor template, including an engineered meganuclease, a zinc finger nuclease, a TALEN, a compact TALEN, a CRISPR system nuclease, or a megaTAL.

For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type Its restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type Hs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system, or other CRISPR system nucleases, are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

The CAR transgene can be inserted at any position within the TCR alpha gene, the TCR beta gene, or the TRAC gene, such that insertion of the transgene results in disrupted expression of the endogenous polypeptide; i.e., the endogenous TCR alpha chain or the endogenous TCR beta chain. In some examples, the CAR transgene can be inserted in the TRAC gene at a meganuclease recognition sequence comprising SEQ ID NO: 1. In particular examples, the transgene is inserted between positions 13 and 14 of SEQ ID NO: 1.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439 and WO 2017/062451. Nuclease-mediated insertion of the donor template can also be accomplished using, for example, an engineered single-chain meganuclease comprising SEQ ID NO: 19.

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element.

The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In some embodiments, mRNA encoding nucleases, are coupled covalently or non-covalently to a nanoparticle or encapsulated within such a nanoparticle using methods known in the art (Sharma, et al. (2014) Biomed Res Int. 2014). A nanoparticle is a nanoscale delivery system whose length scale is <1 mm, preferably <100 nm. Such nanoparticles may be designed using a core composed of metal, lipid, polymer, or biological macromolecule, and multiple copies of the mRNA can be attached to or encapsulated with the nanoparticle core. This increases the copy number of the mRNA that is delivered to each cell and, so, increases the intracellular expression of each engineered nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell surface receptors and the natural ligands (or portions of the natural ligands) for cell surface receptors. In some embodiments, the nanoparticle is a lipid nanoparticle (LNP).

In some embodiments, the mRNA encoding the nucleases are encapsulated within liposomes or complexed using cationic lipids (see, e.g., Lipofectamine™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the target cells.

In some embodiments, mRNA encoding nucleases are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, mRNA encoding recombinant nucleases are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, mRNA encoding nucleases are formulated into an emulsion or a nanoemulsion (e.g., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, mRNA encoding nucleases are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high drug payload capacity. Moreover, display of multiple surface groups can be leveraged to improve stability, reduce nonspecific interactions, and enhance cell-specific targeting and drug release.

In some embodiments, genes encoding a nuclease are delivered using a viral vector. Such vectors are known in the art and include retroviral vectors, lentiviral vectors, adenoviral vectors, and adeno-associated virus (AAV) vectors (reviewed in Vannucci, et al. (2013 New Microbiol. 36:1-22). Recombinant AAV vectors useful in the invention can have any serotype that allows for transduction of the virus into the cell and insertion of the nuclease gene into the cell genome. In particular embodiments, recombinant AAV vectors have a serotype of AAV2 or AAV6. AAV vectors can, in some examples, be single-stranded AAV vectors. AAV vectors can also be self-complementary such that they do not require second-strand DNA synthesis in the host cell (McCarty, et al. (2001) Gene Ther. 8:1248-54).

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, nuclease genes are operably linked to a promoter that drives gene expression preferentially in the target cell (e.g., a T cell).

The invention further provides for the introduction of a donor template (e.g., a template nucleic acid) into a cleavage site in the targeted genes. In some embodiments, the donor template comprises a 5′ homology arm and a 3′ homology arm flanking the transgene (e.g., a CAR transgene) and elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

The transgene encoding the CAR can further comprise additional control sequences. For example, the sequence can include homologous recombination enhancer sequences, Kozak sequences, polyadenylation sequences, transcriptional termination sequences, selectable marker sequences (e.g., antibiotic resistance genes), origins of replication, and the like. Sequences encoding engineered nucleases can also include at least one nuclear localization signal. Examples of nuclear localization signals are known in the art (see, e.g., Lange et al., J. Biol. Chem., 2007, 282:5101-5105).

A donor template comprising the CAR transgene can be introduced into the cell by any of the means previously discussed. In a particular embodiment, the donor template is introduced by way of a viral vector, such as a recombinant AAV vector. Recombinant AAV vectors useful for introducing an exogenous nucleic acid can have any serotype that allows for transduction of the virus into the cell and insertion of the exogenous nucleic acid sequence into the cell genome. In particular embodiments, the recombinant AAV vectors have a serotype of AAV2 or AAV6. AAV vectors can, in some examples, be single-stranded AAV vectors. The recombinant AAV vectors can also be self-complementary such that they do not require second-strand DNA synthesis in the host cell.

In another particular embodiment, the donor template encoding the CAR transgene can be introduced into the cell using a single-stranded DNA template. The single-stranded DNA can comprise the exogenous sequence of interest and, in preferred embodiments, can comprise 5′ and 3′ homology arms to promote insertion of the nucleic acid sequence into the cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, the donor template encoding the CAR transgene can be introduced into the cell by transfection with a linearized DNA template. In some examples, a plasmid DNA can be digested by one or more restriction enzymes such that the circular plasmid DNA is linearized prior to transfection into the cell.

### 5. Pharmaceutical Compositions

The method of the invention provides a pharmaceutical composition comprising a population of human T cells, including a plurality of CAR T cells. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which promote in vivo cell proliferation and engraftment of genetically-modified T cells. Pharmaceutical compositions comprising genetically-modified immune cells of the invention can be administered in the same composition as an additional agent or biological molecule or, alternatively, can be co-administered in separate compositions.

The present disclosure also provides populations of human T cells, comprising a plurality of CAR T cells, described herein for use as a medicament. The present disclosure further provides the use of populations of human T cells, comprising a plurality of CAR T cells, described herein in the manufacture of a medicament for treating a disease in a subject in need thereof. In one such aspect, the medicament is useful for cancer immunotherapy in subjects in need thereof.

### 6. Methods of Administering Populations of Human T Cells

The method of the invention comprises administering to a subject a pharmaceutical composition comprising a population of human cells, wherein the population comprises a plurality of CAR T cells. For example, the pharmaceutical composition administered to the subject can comprise an effective dose of CAR T cells for treatment of a cancer. The administered CAR T cells are able to reduce the proliferation, reduce the number, or kill target cells in the recipient.

Unlike antibody therapies, genetically-modified cells of the present disclosure are able to replicate and expand in vivo, resulting in long-term persistence that can lead to sustained control of a disease. In some embodiments of the invention, the CAR T cells administered as part of the pharmaceutical composition proliferate in vivo for at least one day following administration. In certain examples, the CAR T cells proliferate in vivo between about day 1 and about day 21 following administration of the pharmaceutical composition. Proliferation of CAR T cells can be measured, for example, by techniques known in the art, including determining the number of copies of the CAR transgene per μg of DNA in peripheral blood mononuclear cells, as measured by PCR analysis. In some examples, the number of copies of the CAR transgene per μg of DNA in peripheral blood mononuclear cells is elevated for at least one day, and up to 21 days after administration of the pharmaceutical composition when compared to the number of copies present prior to administration. In further examples, the number of copies of the CAR transgene per μg of DNA in peripheral blood mononuclear cells is elevated to between about 150 copies/μg to about 2100 copies/μg of DNA for at least one day following administration of the pharmaceutical composition.

The subject may be administered the pharmaceutical composition (e.g., comprising human T cells, including CAR T cells), for instance, at a dosage of from about 3×10⁴ to about 1×10⁷ CAR T cells/kg. In some embodiments, the subject is administered a pharmaceutical composition described herein at a dosage of about 1×10⁵ to about 1×10⁷ CAR T cells/kg. In some embodiments, the subject is administered a pharmaceutical composition described herein at a dosage of about 3×10⁵ to about 1×10⁷ CAR T cells/kg. In some embodiments, the subject is administered a pharmaceutical composition described herein at a dosage of about 3×10⁴ to about 6×10⁶ CAR T cells/kg. In some embodiments, the subject is administered a pharmaceutical composition described herein at a dosage of about 3×10⁵ to about 6×10⁶ CAR T cells/kg. In some embodiments, the subject is administered a pharmaceutical composition described herein at a dosage of about 3×10⁵ to about 3×10⁶ CAR T cells/kg. In some embodiments, the subject is administered a pharmaceutical composition described herein at a dosage of about 3×10⁴ CAR T cells/kg. In some embodiments, the subject is administered a pharmaceutical composition described herein at a dosage of about 3×10³ CAR T cells/kg. In some embodiments, the subject is administered a pharmaceutical composition described herein at a dosage of about 5×10³ CAR T cells/kg. In some embodiments, the subject is administered a pharmaceutical composition described herein at a dosage of about 1×10⁶ CAR T cells/kg. In some embodiments, the subject is administered a pharmaceutical composition described herein at a dosage of about 1.5×10⁶ CAR T cells/kg. In some embodiments, the subject is administered a pharmaceutical composition described herein at a dosage of about 2×10⁶ CAR T cells/kg. In some embodiments, the subject is administered a pharmaceutical composition described herein at a dosage of about 2.5×10⁶ CAR T cells/kg. In some embodiments, the subject is administered a pharmaceutical composition described herein at a dosage of about 3×10⁶ CAR T cells/kg. In some embodiments, the subject is administered a pharmaceutical composition described herein at a dosage of about 3.5×10⁶ CAR T cells/kg. In some embodiments, the subject is administered a pharmaceutical composition described herein at a dosage of about 4×10⁶ CAR T cells/kg. In some embodiments, the subject is administered a pharmaceutical composition described herein at a dosage of about 4.5×10⁶ CAR T cells/kg. In some embodiments, the subject is administered a pharmaceutical composition described herein at a dosage of about 5×10⁶ CAR T cells/kg. In some embodiments, the subject is administered a pharmaceutical composition described herein at a dosage of about 5.5×10⁶ CAR T cells/kg. In some embodiments, the subject is administered a pharmaceutical composition described herein at a dosage of about 6×10⁶ CAR T cells/kg.

The subject may be administered a first dose of the pharmaceutical composition and a second dose of the pharmaceutical composition. In some cases, the second dose of the pharmaceutical composition is administered without re-administration of the lymphodepletion regimen; i.e., a first and second dose of the pharmaceutical composition is administered following a single lymphodepletion regimen. In some examples, the second dose of the pharmaceutical composition is administered 1, 2, 3, 4, 5, 6, 7, 8, 9, 10, 11, 12, 13, 14, 15, 16, 17, 18, 19, 20, or 21 days following administration of the first dose of the pharmaceutical composition. In certain examples, the second dose of the pharmaceutical composition is administered 10 days following administration of the first dose of the pharmaceutical composition. In some examples, the second dose of the pharmaceutical composition is administered at the same dose of CAR T cells/kg as administered in the first dose of the pharmaceutical composition. In other examples, the second dose of the pharmaceutical composition is administered at a different dose of CAR T cells/kg than administered in the first dose of the pharmaceutical composition.

In some examples, the subject is administered a first dose of the pharmaceutical composition, a second dose of the pharmaceutical composition, and a third dose of the pharmaceutical composition. In certain examples, the second dose of the pharmaceutical composition and the third dose of the pharmaceutical composition are administered without re-administration of the lymphodepletion regimen; i.e., a first dose, a second dose, and a third dose are administered following a single lymphodepletion regimen. In some examples, the second dose of the pharmaceutical composition is administered 1, 2, 3, 4, 5, 6, 7, 8, 9, 10, 11, 12, 13, 14, 15, 16, 17, 18, 19, 20, or 21 days following administration of the first dose of the pharmaceutical composition, and the third dose of the pharmaceutical composition is administered 1, 2, 3, 4, 5, 6, 7, 8, 9, 10, 11, 12, 13, 14, 15, 16, 17, 18, 19, 20, or 21 days following administration of the second dose of the pharmaceutical composition. In certain examples, the second dose of the pharmaceutical composition is administered 10 days following administration of the first dose of the pharmaceutical composition, and the third dose of the pharmaceutical composition is administered 4 days following administration of the second dose of the pharmaceutical composition. In some examples, the second dose of the pharmaceutical composition and the third dose of the pharmaceutical composition are each administered at the same dose of CAR T cells/kg as administered in the first dose of the pharmaceutical composition. In some examples, the second dose of the pharmaceutical composition is administered at the same dose of CAR T cells/kg as administered in the first dose of the pharmaceutical composition, and the third dose of the pharmaceutical composition is administered at a different dose of CAR T cells/kg than administered in the first dose of the pharmaceutical composition. In some examples, the second dose of the pharmaceutical composition is administered at a different dose of CAR T cells/kg than administered in the first dose of the pharmaceutical composition, and the third dose of the pharmaceutical composition is administered at the same dose of CAR T cells/kg as administered in the first dose of the pharmaceutical composition. In some examples, the second dose of the pharmaceutical composition is administered at a different dose of CAR T cells/kg than administered in the first dose of the pharmaceutical composition, and the third dose of the pharmaceutical composition is administered at the same dose of CAR T cells/kg as administered in the first dose of the pharmaceutical composition. In some examples, the second dose of the pharmaceutical composition and the third dose of the pharmaceutical composition are administered at the same dose of CAR T cells/kg, and are administered at a different dose of CAR T cells/kg than administered in the first dose of the pharmaceutical composition.

In certain examples of the methods, the subject is re-administered both the lymphodepletion regimen and the pharmaceutical composition. In some examples, re-administration of the lymphodepletion regimen and the pharmaceutical composition occurs following a partial response or complete response to the first lymphodepletion regimen and pharmaceutical composition with subsequent progressive disease. In some examples, re-administration of the lymphodepletion regimen and the pharmaceutical composition occurs following no response to the first lymphodepletion regimen and pharmaceutical composition and subsequent progressive disease. In some examples, re-administration of the lymphodepletion regimen and the pharmaceutical composition occurs in subjects having a cancer that remains positive for the cancer cell antigen targeted by the CAR T cells (e.g., are positive for CD19, CD20, or BCMA). In some examples, re-administration of the lymphodepletion regimen and the pharmaceutical composition occurs about 2 weeks, 4 weeks, 6, weeks, 8 weeks, 10 weeks, 12, weeks, 14, weeks, 16 weeks, 18 weeks, 20 weeks, 22 weeks, 24 weeks, or more after the first administration of the lymphodepletion regimen and pharmaceutical composition. In some examples, the lymphodepletion regimen is re-administered at the same doses and/or schedule as the first administration. In some examples, the lymphodepletion regimen is re-administered at different doses and/or a different schedule as the first administration. In some examples, the lymphodepletion regimen is re-administered according to any of the doses and/or dosing schedules described herein for administration of the first lymphodepletion regimen. In some examples, the pharmaceutical composition is re-administered at the same doses and/or schedule as the first administration. In some examples, the pharmaceutical composition is re-administered at different doses and/or a different schedule as the first administration. In certain examples, the pharmaceutical composition is re-administered at a higher dose than the first administration. In some examples, the pharmaceutical composition is re-administered at a dose of about 1×10⁶ CAR T cells/kg. In some examples, the pharmaceutical composition is re-administered at a dose of about 2×10⁶ CAR T cells/kg. In some examples, the pharmaceutical composition is re-administered at a dose of about 3×10⁶ CAR T cells/kg. In some examples, the pharmaceutical composition is re-administered at a dose of about 4×10⁶ CAR T cells/kg. In some examples, the pharmaceutical composition is re-administered at a dose of about 5×10⁶ CAR T cells/kg. In some examples, the pharmaceutical composition is re-administered at a dose of about 6×10⁶ CAR T cells/kg. In some examples, a first dose and a second dose, and optionally a third dose, of the pharmaceutical composition are re-administered according to any of the doses and/or dosing schedules described herein for administration of a first dose and a second dose, or for administration of a first dose, a second dose, and a third dose.

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified immune cells or populations thereof described herein is administered at a dosage of 10⁴ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

Examples of possible routes of administration of compositions comprising genetically-modified cells or lymphodepletion regimens described herein include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some embodiments, a pharmaceutical composition of the present disclosure, which comprises CAR T cells, targets a cancer cell antigen (i.e., an antigen expressed on the surface of a cancer cell) for the purposes of treating cancer. Such cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In some embodiments of the invention, the method of immunotherapy is provided to the subject after prior immunotherapy, including after prior CAR T therapy.

In some embodiments, administration of the pharmaceutical compositions of the present disclosure (comprising human T cells, including CAR T cells), reduce at least one symptom of a cancer. Symptoms of cancers are well known in the art and can be determined by known techniques.

In some of these embodiments wherein cancer is treated, the subject can be further administered an additional therapeutic agent or treatment, including, but not limited to gene therapy, radiation, surgery, or a chemotherapeutic agent(s) (i.e., chemotherapy).

In some embodiments of the invention, the serum concentrations of certain cytokines are elevated following administration of the pharmaceutical compositions disclosed herein. For example, the serum concentration of C-reactive protein, ferritin, IL-6, interferon gamma, or any combination thereof, can be elevated compared to the concentration at day 0 for at least one day following administration of the pharmaceutical composition.

According to the invention, a subject treated by the methods can achieve a partial response, or a complete response, to the method of immunotherapy. In some examples, the partial response or complete response can be maintained through at least 28 days after administration of the pharmaceutical compositions described herein.

### 7. Variants

The present invention encompasses variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein. Such variants may result, for example, from human manipulation. Biologically active variants of polypeptides described herein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (e.g., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Allogeneic CD19 CAR T Therapy in Non-Hodgkin Lymphoma and B-Cell Acute Lymphoblastic Leukemia

### A. Background

PBCAR0191 is an off-the-shelf allogeneic CD19-targeted chimeric antigen receptor (CAR) T cell product derived from qualified donor T cells that have been genetically edited to remove the expression of the endogenous T cell receptor (TCR) and insert the CAR in the same locus. The goal is to achieve anti-tumor effect and reduce the possibility of graft-versus-host disease (GvHD) when it is administered to human leukocyte antigen (HLA)-mismatched patients with CD19 expressing B-cell malignancies.

This is a Phase 1/2a, nonrandomized, open-label, parallel assignment, single-dose, dose-escalation, and dose-expansion study to evaluate the safety and clinical activity of PBCAR0191 in adults with r/r B-ALL (Cohort A) and in adults with r/r B-cell NHL (Cohort N).

This is a multicenter, nonrandomized, open-label, parallel assignment, single-dose, dose-escalation, and dose-expansion study to evaluate the safety and tolerability, find an appropriate dose to optimize safety and efficacy, and evaluate clinical activity of PBCAR0191 in subjects with r/r B-ALL and r/r/NHL. Before initiating PBCAR0191, subjects will be administered lymphodepletion chemotherapy composed of fludarabine and cyclophosphamide. At Day 0 of the Treatment Period, subjects will receive a single intravenous (IV) infusion of PBCAR0191. All subjects are monitored during the treatment period through Day 28. All subjects who receive a dose of PBCAR0191 will be followed in a separate long-term follow-up (LTFU) study for 15 years after exiting this study.

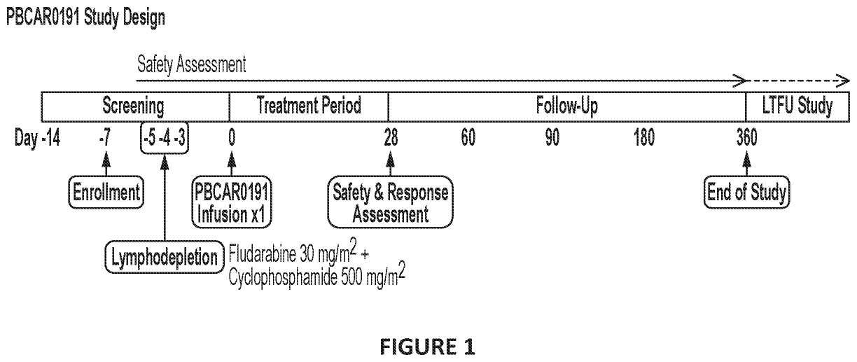

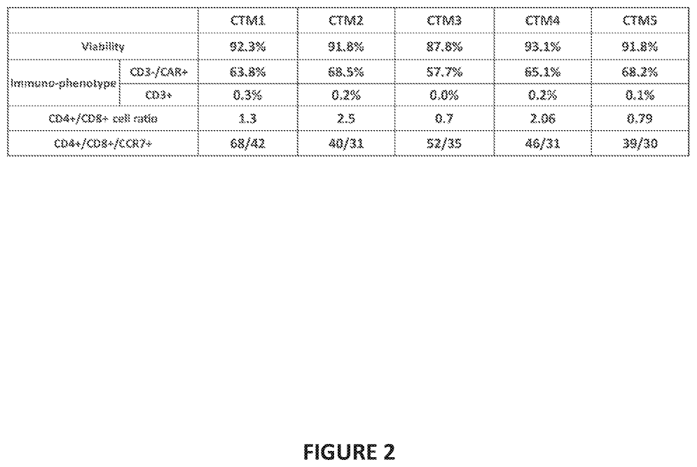

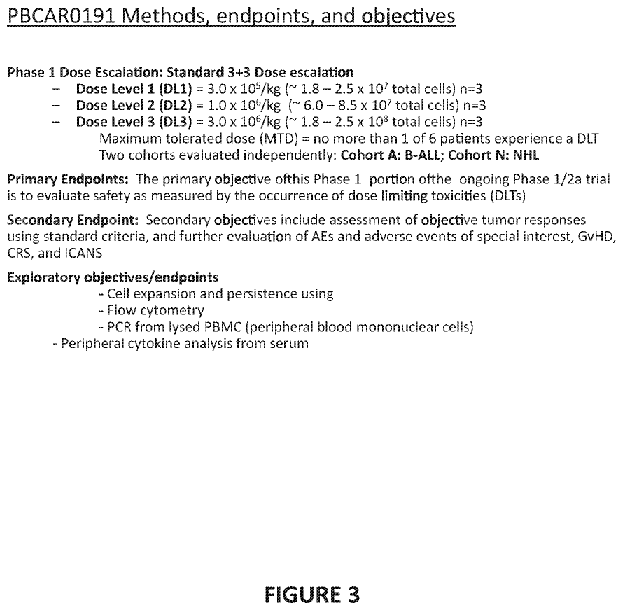

In each cohort (NHL and B-ALL), 3 escalating dose groups will be enrolled and treated sequentially. Within each dose group, up to 6 subjects will be treated with a single dose of PBCAR0191 using a standard 3+3 design. The starting dose of PBCAR0191 will be 3×10⁵ CAR T cells/kg body weight. Subsequent dose groups will be treated with escalating doses to a maximum dose of 3×10⁶ CAR T cells/kg. In the absence of DLTs, the dose will be increased using a fixed dose scheme. Results in this example were obtained following treatment of patients with the clinical trial material described in FIG. 2 as CTM1, CTM2, and CTM3. Phase 1 Dose Escalation:
Dose Level −1 (DL-1)=3.0×10⁴/kg (˜0.18-0.25×10⁷ total cells)
Dose Level 1 (DL1)=3.0×10⁵/kg (˜1.8-2.5×10⁷ total cells)
Dose Level 2 (DL2)=1.0×10⁶/kg (˜6.0-8.5×10⁷ total cells)
Dose Level 3 (DL3)=3.0×10⁶/kg (˜1.8-2.5×10⁸ total cells)
Dose Level 3b (DL3b)=3.0×10⁶/kg (˜1.8-2.5×10⁸ total cells) (administered as 3 administrations of 1.0×10⁶/kg)
Dose Level 4 (DL4)=6.0×10⁶/kg (˜3.6-5.0×10⁸ total cells) (administered as 2 administrations of 3.0×10⁶/kg, i.e., was “split” into two doses of 3.0×10⁶/kg)
Dose Level 5 (DL5)=9.0×10⁶/kg (˜5.4-7.5×10⁸ total cells) (administered as 3 administrations of 3.0×10⁶/kg)
Maximum tolerated dose (MTD)=no more than 1 of 6 patients experience a DLT
   The lymphodepletion regimen includes the administration of cyclophosphamide (500 mg/m²) and fludarabine (30 mg/m²) on days −5 to −3 prior to PBCAR0191 infusion. The PBCAR0191 study design is illustrated in FIG. 1. Exemplary clinical trial material batches used during the PBCAR0191 study are described in FIG. 2.
   Primary outcome measures include:
Maximum Tolerated Dose (MTh) [Time Frame: Day 1-Day 28]
To determine the maximum tolerated dose (MTD), which is defined as the dose level at which fewer than 33% of patients experience a dose limiting toxicity (DLT) using a 3+3 strategy.
Number of Participants with Dose Limiting Toxicity(ies) [Time Frame: 1 year]
To assess adverse events as dose limiting toxicities as defined by the protocol and CTCAE v5.0.
   Secondary outcome measures include:
Objective Response Rate of Patients [Time Frame: 1 year]
To assess clinical activity as response in B-ALL by the NCCN Guidelines on ALL (NCCN, 2017) and in NHL by the revised Lugano Classification (Cheson et al., 2016), both reported as objective response rate.
Area Under the Curve [AUC] [Time Frame: Up to 1 year]
To evaluate Area Under the Curve [AUC] of PBCAR0191 in patients tested.

Primary Endpoints: The primary objective of this Phase 1 portion of the ongoing Phase 1/2a trial is to evaluate safety as measured by the occurrence of dose limiting toxicities (DLTs).

Secondary Endpoint: Secondary objectives include assessment of objective tumor responses using standard criteria, and further evaluation of AEs and adverse events of special interest, GvHD, CRS, and ICANS.

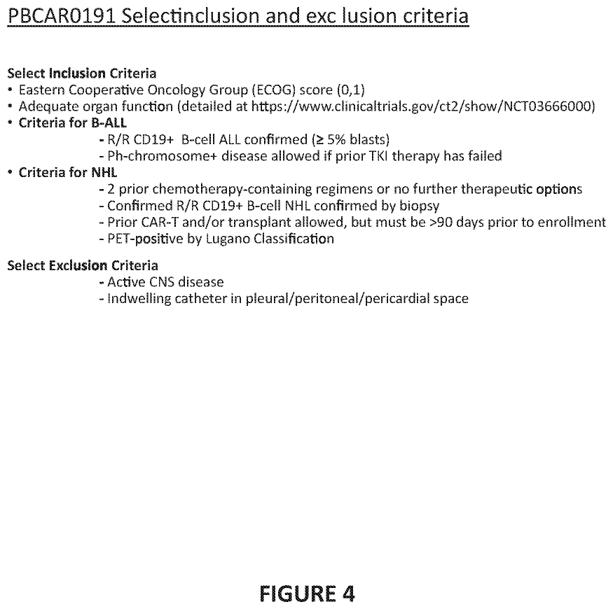

Exploratory objectives/endpoints:
Cell expansion and persistence using
   Flow cytometry
   PCR from lysed PBMC (peripheral blood mononuclear cells)
Peripheral cytokine analysis from serum
   Ages eligible for study: 18 Years and older (Adult, Older Adult)
   Sexes eligible for study: All
   Accepts healthy volunteers: No
   Key inclusion criteria (see also, FIG. 4)

### Criteria for B-ALL:

Relapsed or refractory CD19+ B-cell acute lymphoblastic leukemia (B-ALL).
Philadelphia chromosome positive (Ph+) disease can be eligible if they are intolerant to tyrosine kinase inhibitor (TKI) therapy or if they have relapsed/refractory disease.

### Criteria for NHL:

r/r CD19+ B-cell NHL that is histologically confirmed by archived tumor biopsy tissue from last relapse and corresponding pathology report. The following types of lymphoma are included:
   Diffuse large B-cell lymphoma (DLBCL) including Richter's transformation
   Primary mediastinal B-cell lymphoma (PMBL)
   FL including Grade 3B or transformed FL
   High-grade B-cell lymphoma
   Small lymphocytic lymphoma (SLL)
   Mantle cell lymphoma (MCL)
Received at least 2 prior chemotherapy-containing regimens. Subjects with SLL must have previously failed at least 2 lines of chemotherapy/immunotherapy that included ibrutinib and idelalisib plus rituximab.
Measurable or detectable disease according to the Lugano Classification.
Criteria for both B-ALL and NHL:
Eastern Cooperative Oncology Group performance status score of 0 or 1.
An estimated life expectancy of at least 12 weeks according to the investigator's judgment.
Seronegative for human immunodeficiency virus antibody (e.g., intact immune function).
Subject has adequate bone marrow, renal, hepatic, pulmonary, and cardiac function defined as:
   Estimated glomerular filtration rate (eGFR)>50 mL/min/1.73 m².
   Alanine aminotransferase (ALT) and aspartate aminotransferase (AST) levels both ≤3 times of upper limit of normal, unless there is suspected disease in the liver.
   Total bilirubin <2.0 mg/dL, except in subjects with Gilbert's syndrome.
   Platelet count ≥30,000/μL (platelet transfusions acceptable).
   Left ventricular ejection fraction >45% as assessed by echocardiogram (ECHO) or multiple gated acquisition scan performed within 1 month before starting lymphodepleting chemotherapy. ECHO results performed within 6 months before Screening and at least 28 days after the last cancer treatment may be acceptable if the subject has not received any treatment with cardiotoxicity risks.
   No clinically significant evidence of pericardial effusion or pleural effusion.
   Baseline oxygen saturation >92% on room air.

### Key Exclusion Criteria

### Criteria for B-ALL:

Burkitt cell (L3 ALL) or mixed-lineage acute leukemia.
Active CNS leukemia.

### Criteria for NHL:

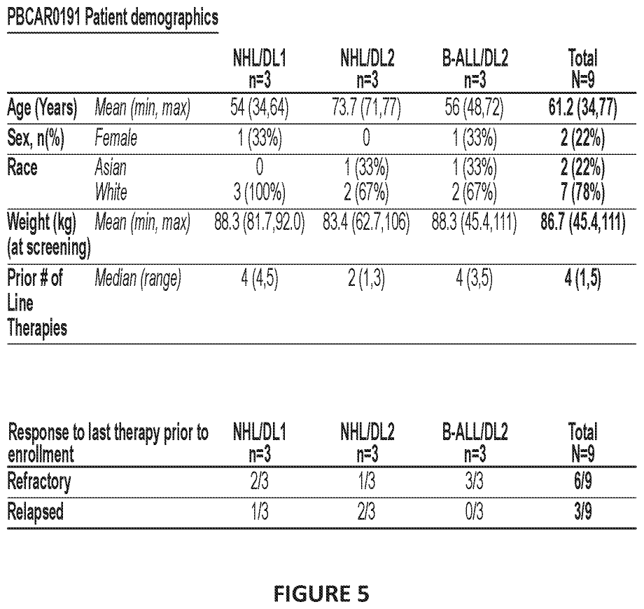

Active hemolytic anemia.
Active CNS lymphoma.
Criteria for B-ALL and NHL:
Previous malignancy, besides the malignancies of inclusion (B-ALL or NHL), that has a high risk of relapse in the next 2 years.
Uncontrolled and serious fungal, bacterial, viral, protozoal, or other infection.
Any form of primary immunodeficiency.
Active hepatitis B or C.
Uncontrolled cardiovascular disease.
Hypertension crisis or hypertensive encephalopathy within 3 months.
Concomitant genetic syndrome or any other known bone marrow failure syndrome.
Active uncontrolled autoimmune disease requiring active immunosuppression (excluding subjects needing steroids for physiologic replacement).
Received stem cell transplant within 90 days.
Active GvHD symptoms.
Received systemic biologic agent within 30 days or 5 half-lives.
Received systemic immunostimulatory agent within 30 days or 5 half-lives.
Radiotherapy within 4 weeks determined on a case-by-case basis.
Presence of pleural/peritoneal/pericardial catheter.
Received live vaccine within 4 weeks before Screening.
Current use of any anticoagulant or antiplatelet therapy.
Additional criteria apply
   Patient demographics for PBCAR0191 are shown in FIG. 5.

### B. Results

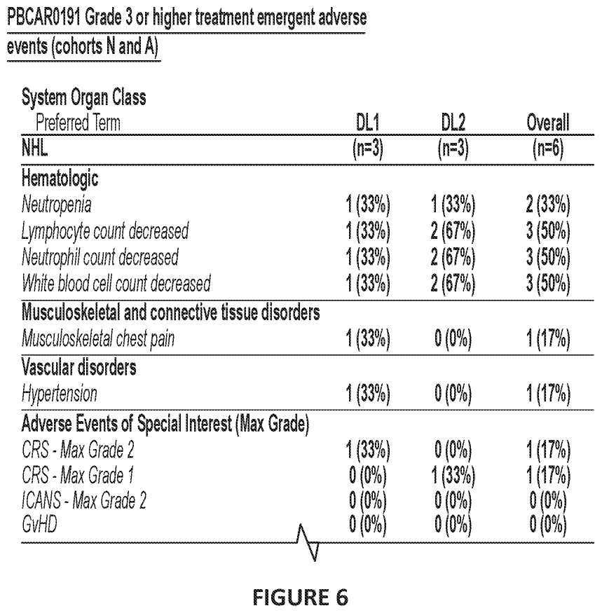

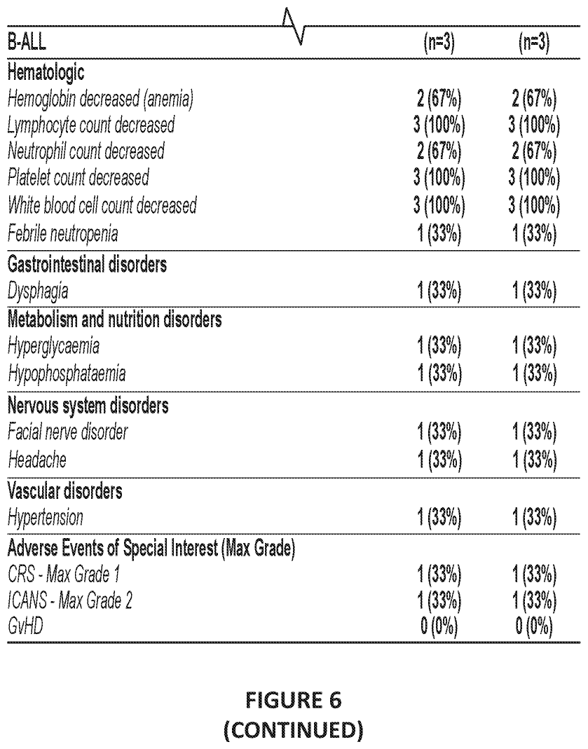

PBCAR0191 Grade 3 or higher treatment adverse events (cohorts N and A) are shows in FIG. 6.

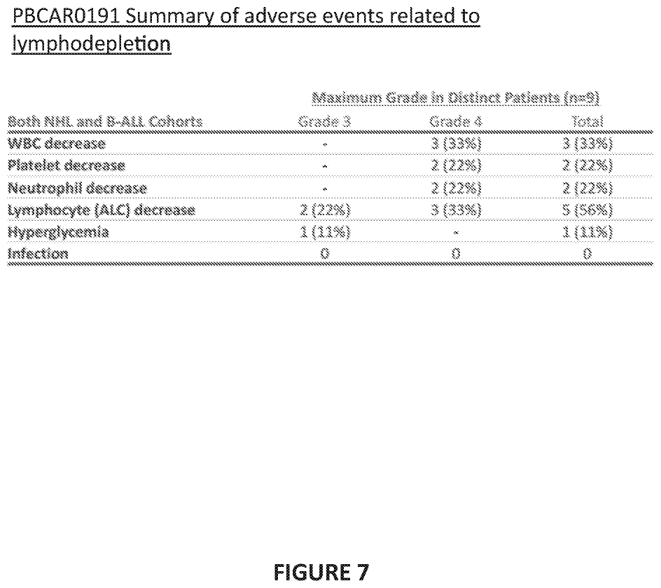

A summary of PBCAR0191 adverse events related to lymphodepletion is shown in FIG. 7.

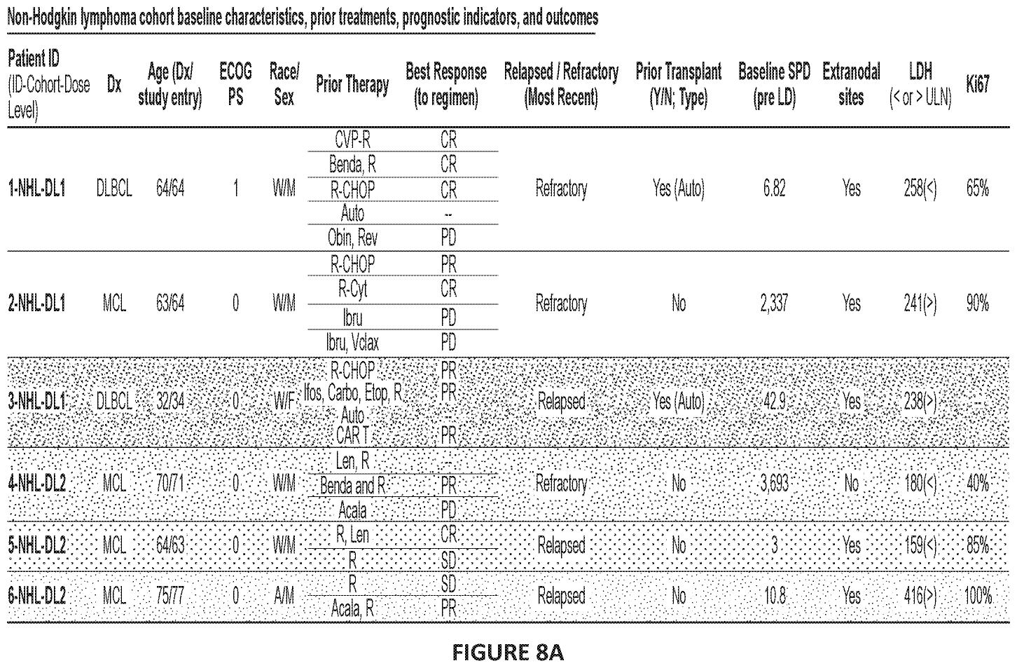

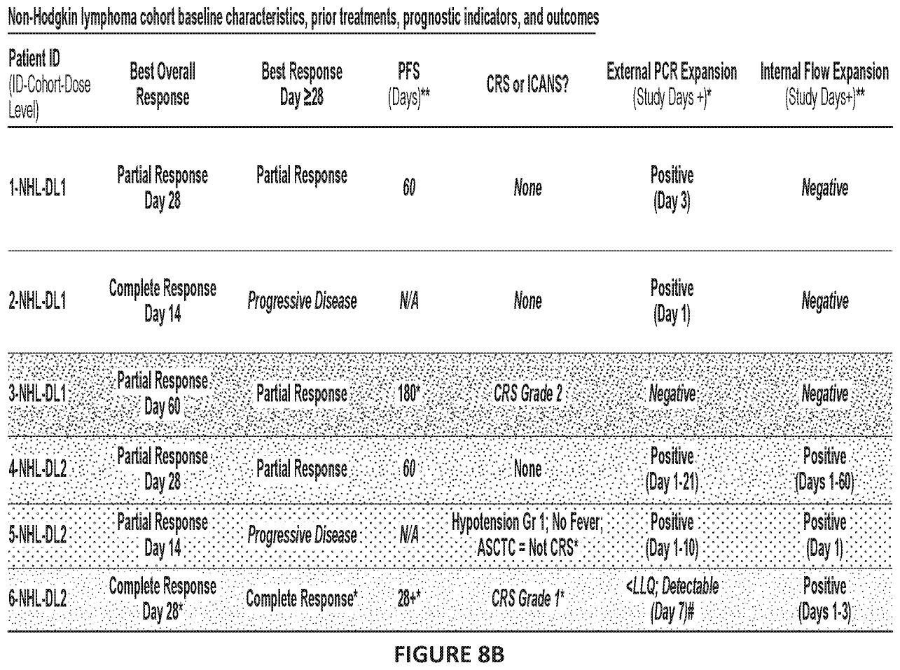

A summary of the non-Hodgkin lymphoma cohort baseline characteristics, prior treatments, prognostic indicators, and outcomes is shown in FIG. 8.

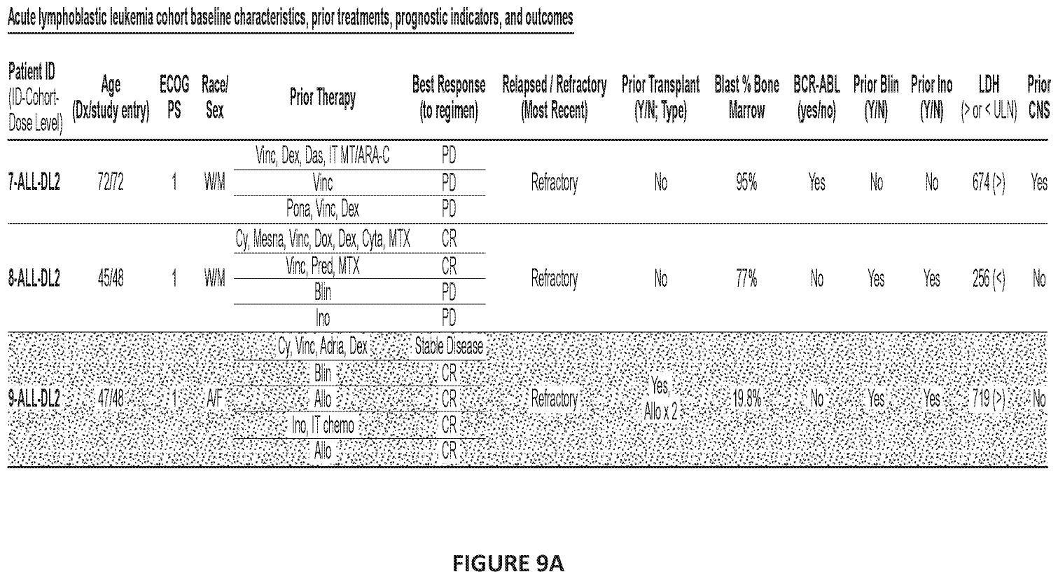

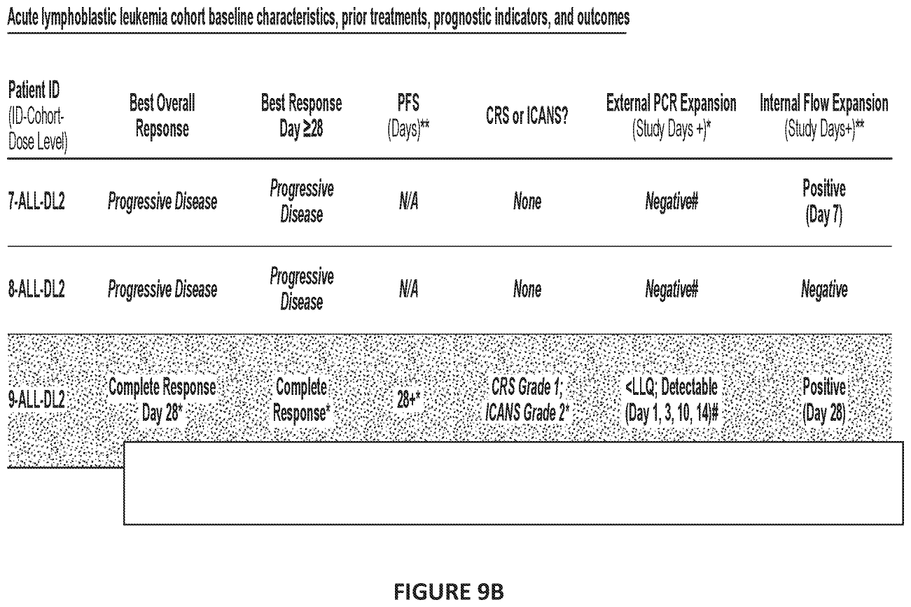

A summary of the acute lymphoblastic leukemia cohort baseline characteristics, prior treatments, prognostic indicators, and outcomes is shown in FIG. 9.

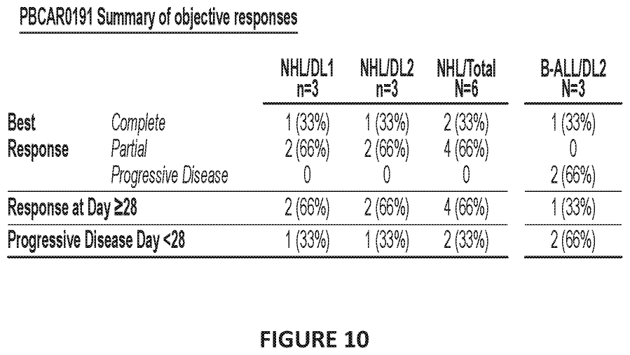

A summary of objective responses is shown in FIG. 10.

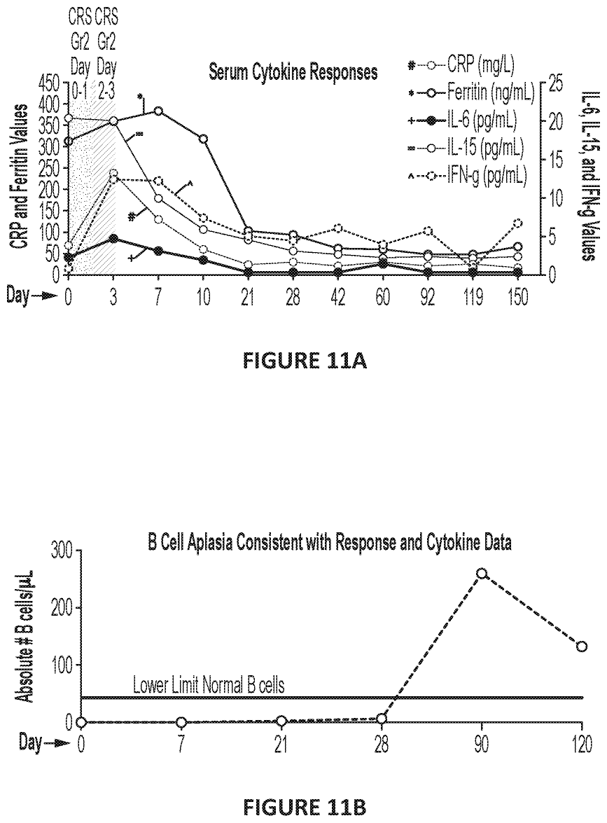

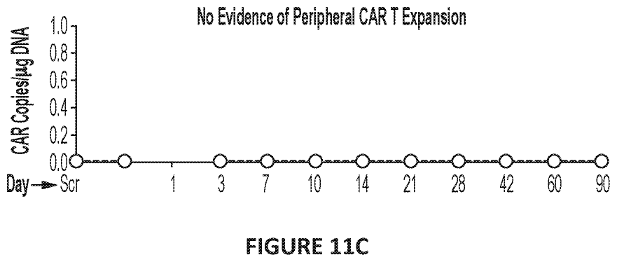

A summary of serum cytokine responses measured in Patient 3-NHL-DL1 is shown in FIG. 11A. Cytokines measured include C-reactive protein (mg/L), ferritin (ng/mL), IL-6 (pg/mL), IL-15 (pg/mL), and interferon gamma (pg/mL). Days following administration of PBCAR0191 are shown on the X-axis. Cytokine concentrations are shown on the Y-axes.

A summary of B cell aplasia measured in Patient 3-NHL-DL1 is shown in FIG. 11B. Days following administration of PBCAR0191 are shown on the X-axis. The absolute number of B cells/μL is shown on the Y-axis.

Peripheral CAR T expansion measured in Patient 3-NHL-DL1 is shown in FIG. 11C. Days following administration of PBCAR0191 are shown on the X-axis. The number of copies of the CAR transgene measured per μg of DNA isolated from peripheral blood mononuclear cells is shown on the Y-axis.

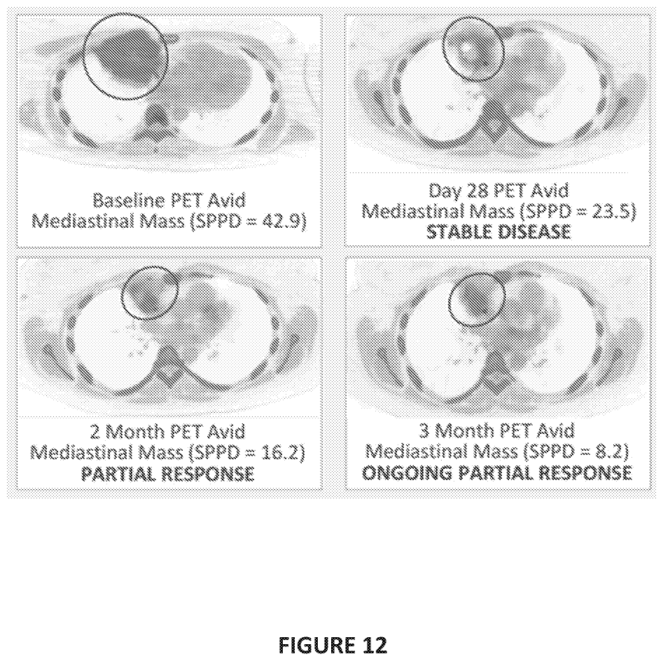

PET scans of Patient 3-NHL-DL1, taken at baseline, day 28, 2 months, and 3 months after administration of PBCAR0191, are shown in FIG. 12.

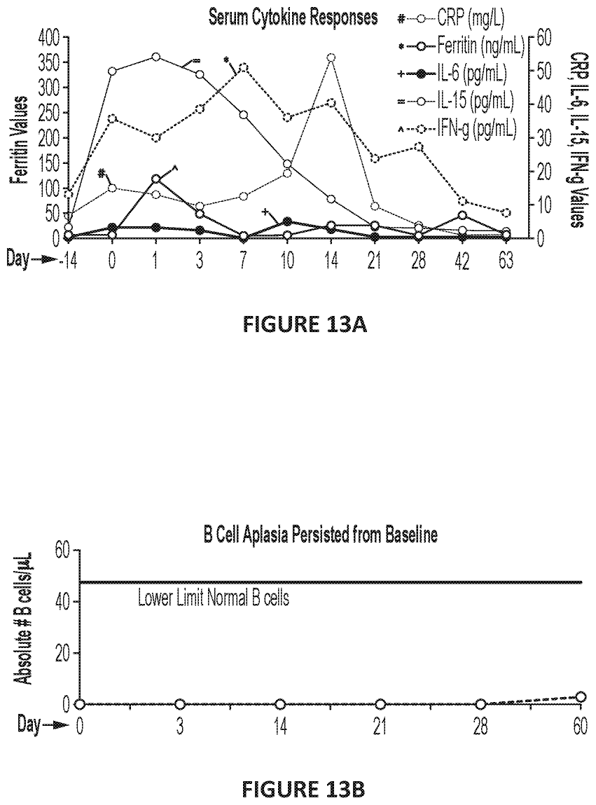

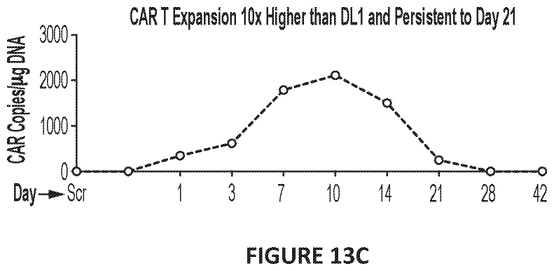

A summary of serum cytokine responses measured in Patient 4-NHL-DL2 is shown in FIG. 13A. Cytokines measured include C-reactive protein (mg/L), ferritin (ng/mL), IL-6 (pg/mL), IL-15 (pg/mL), and interferon gamma (pg/mL). Days following administration of PBCAR0191 are shown on the X-axis. Cytokine concentrations are shown on the Y-axes.

A summary of B cell aplasia measured in Patient 4-NHL-DL2 is shown in FIG. 13B. Days following administration of PBCAR0191 are shown on the X-axis. The absolute number of B cells/μL is shown on the Y-axis.

Peripheral CAR T expansion measured in Patient 4-NHL-DL2 is shown in FIG. 13C. Days following administration of PBCAR0191 are shown on the X-axis. The number of copies of the CAR transgene measured per μg of DNA isolated from peripheral blood mononuclear cells is shown on the Y-axis.

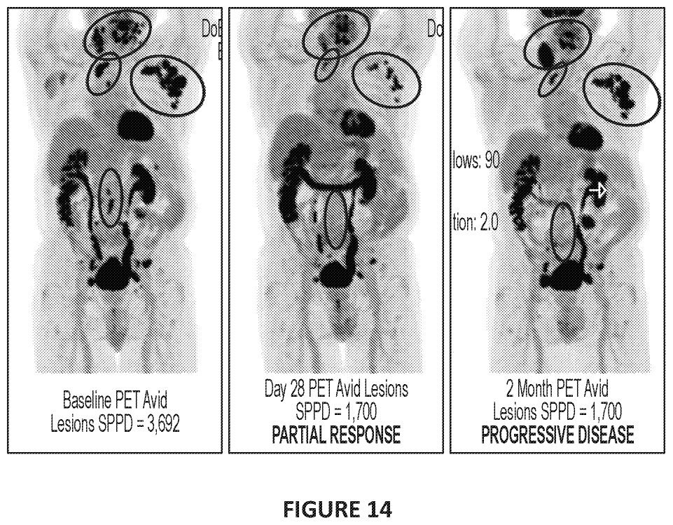

PET scans of Patient 4-NHL-DL2, taken at baseline, day 28, and 2 months after administration of PBCAR0191, are shown in FIG. 14.

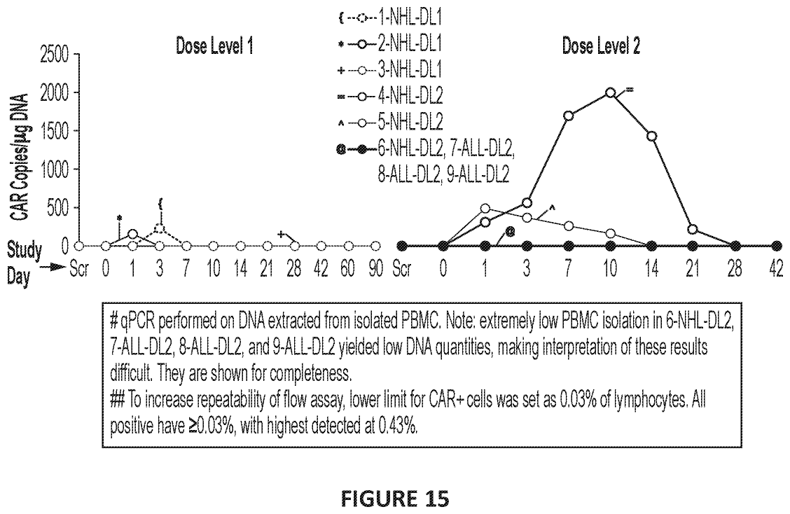

Peripheral CAR T expansion measured in multiple patients in the NHL cohort at dose level 1 and dose level 2 is shown in FIG. 15. Days following administration of PBCAR0191 are shown on the X-axis. The number of copies of the CAR transgene measured per μg of DNA in isolated from peripheral blood mononuclear cells is shown on the Y-axis.

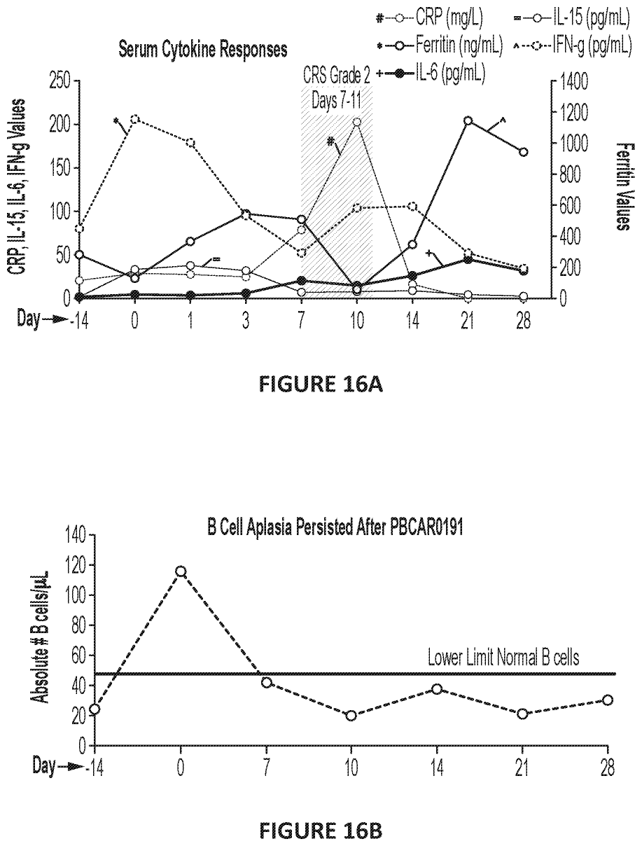

A summary of serum cytokine responses measured in Patient 6-NHL-DL2 is shown in FIG. 16A. Cytokines measured include C-reactive protein (mg/L), ferritin (ng/mL), IL-6 (pg/mL), IL-15 (pg/mL), and interferon gamma (pg/mL). Days following administration of PBCAR0191 are shown on the X-axis. Cytokine concentrations are shown on the Y-axes.

A summary of B cell aplasia measured in Patient 6-NHL-DL2 is shown in FIG. 16B. Days following administration of PBCAR0191 are shown on the X-axis. The absolute number of B cells/μL is shown on the Y-axis.

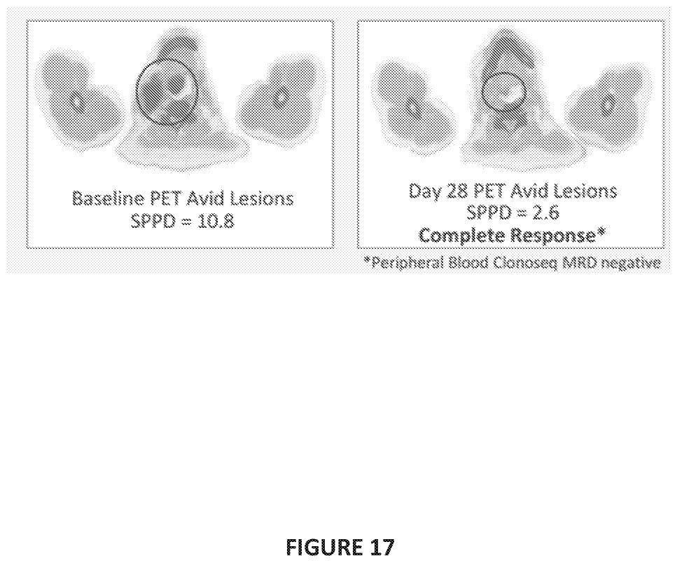

PET scans of Patient 6-NHL-DL2, taken at baseline and day 28 after administration of PBCAR0191, are shown in FIG. 17.

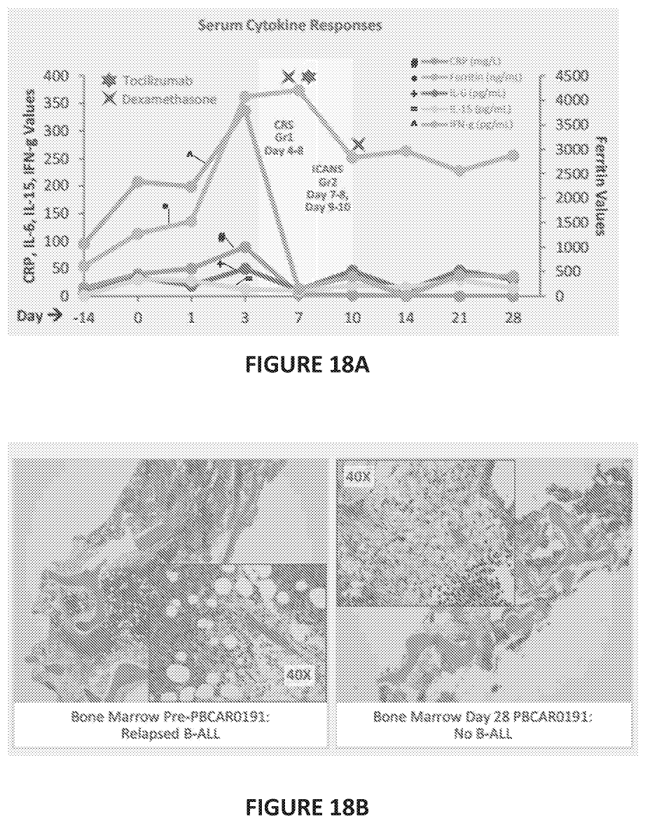

A summary of serum cytokine responses measured in Patient 9-ALL-DL2 is shown in FIG. 18A. Cytokines measured include C-reactive protein (mg/L), ferritin (ng/mL), IL-6 (pg/mL), IL-15 (pg/mL), and interferon gamma (pg/mL). Days following administration of PBCAR0191 are shown on the X-axis. Cytokine concentrations are shown on the Y-axes.

Images of bone marrow demonstrating the presence of B-ALL blasts at baseline and absence of B-ALL blasts at day 28 following administration of PBCAR0191 are shown in FIG. 18B.

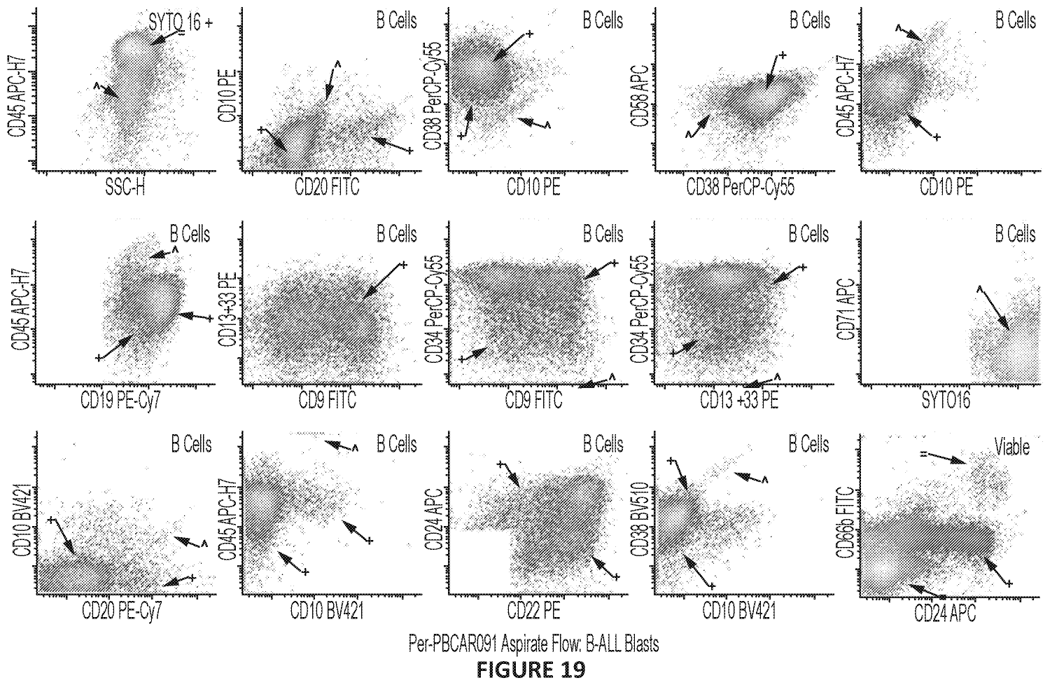

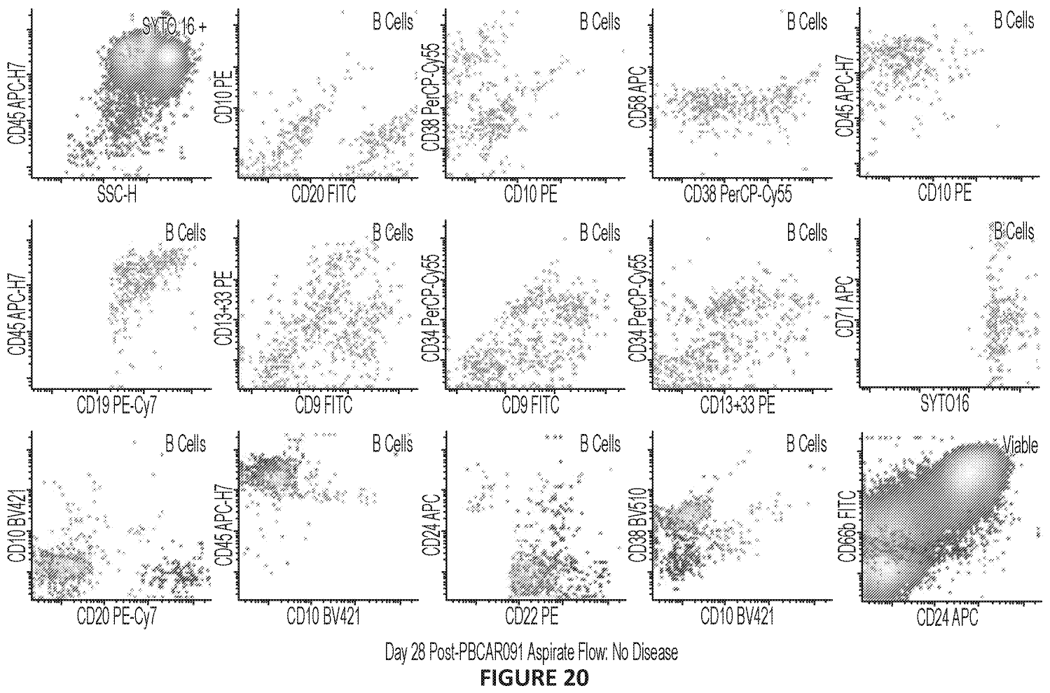

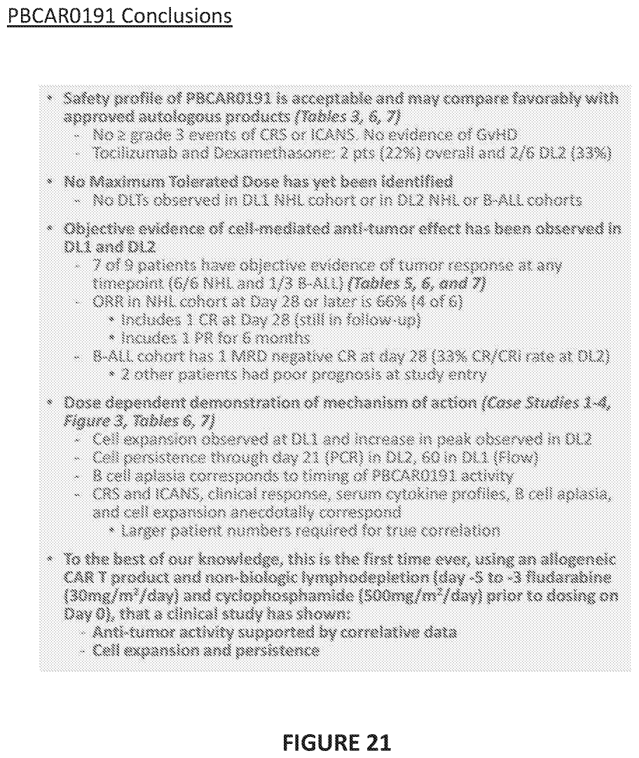

Flow cytometry measuring B-ALL blasts in aspirate at baseline and day 28 following administration of PBCAR0191 are shown in FIGS. 19 and 20, respectively.

Conclusions drawn from PBCAR0191 data disclosed herein are shown in FIG. 21.

### Example 2

### Interim Results From Clinical Trial in Relapsed/Refractory Non-Hodgkin Lymphoma (NHL) and B-Cell Acute Lymphoblastic Leukemia (B-ALL)

### Summary

Interim Results from PBCAR0191 Phase 1/2a trial in Relapsed/Refractory Non-Hodgkin Lymphoma (NHL) and B-cell Acute Lymphoblastic Leukemia (B-ALL) showed acceptable tolerability and safety profile in 27 patients with no graft versus host disease (GvHD), Grade >3 cytokine release syndrome (CRS) or neurotoxicity (ICANS). PCBAR0191 demonstrated durability of response to 11 months. PBCAR0191 with enhanced lymphodepletion improved responses with objective response rate (ORR) of 83% (5/6) in NHL and B-ALL. 75% (3/4) of NHL patients had complete response at day 28. Peak cell expansion increased approximately 95-fold in NHL patients with enhanced lymphodepletion.

### Trial Design:

Interim data from the Phase 1/2a study of PBCAR0191 includes data from 27 patients: 16 patients with R/R NHL and 11 patients with aggressive R/R B-ALL from multiple dose levels. In the NHL cohort, 81% of patients had stage III/IV disease, 63% of patients had extranodal disease, 63% had >4 courses of prior treatment, 100% of reported subjects had aggressive lymphomas (56% had diffuse large B-cell lymphoma, 31% had Mantle Cell Lymphoma), and 25% had prior autologous CAR T cell therapy. In the B-ALL cohort, 55% of patients had >20% blasts burden at baseline, 82% had 4+ courses of prior treatment and 45% had prior allogeneic stem cell transplant. Results in this example were obtained following treatment of patients with the clinical trial material described in FIG. 2 as CTM1, CTM2, CTM3, CTM4, and CTM5.

PBCAR0191 treatment at dose level 1 (DL1) (3×10⁵ CAR T cells/kg), dose level 2 (DL2) (1×10⁶ CAR T cells/kg), dose level 3 (DL3) (3×10⁶ CAR T cells/kg) and split-dose dose level 4 (DL4) (2 doses at 3×10⁶ CAR T cells/kg, wherein the first dose was administered on day 0 and the second dose on day 10) employed a lymphodepletion regimen consisting of fludarabine (30 mg/m²/day for 3 days) plus cyclophosphamide (500 mg/m²/day for 3 days), each administered from day −5 to day −3. PBCAR0191 was also dosed at DL3 (3×10⁶ CAR T cells/kg) with an enhanced lymphodepletion regimen consisting of fludarabine (30 mg/m²/day), administered on days −6 to −3, and cyclophosphamide (1000 mg/m²/day), administered on days −5 to −3.

In some cases, the lymphodepletion regimen was re-administered at the same dose and schedule as the first administration. In such cases, a second dose of PBCAR0191 was administered to patients following the re-administration of the lymphodepletion regimen.

### Response Rates and Cell Expansion:

For this interim analysis, in which patients received either standard or enhanced lymphodepletion (LD), response and cell expansion rates across R/R NHL and R/R B-ALL patient cohorts are as follows. An 83% ORR at day 28 or beyond for patients across NHL (n=4) and B-ALL (n=2) who received PBCAR0191 at DL3 when coupled with enhanced lymphodepletion. At Day 28, 75% (3/4) of NHL patients who received PBCAR0191 at DL3 with enhanced lymphodepletion achieved a CR. Meanwhile, 33% of NHL patients (n=12) across DL1, DL2, and DL3 using standard lymphodepletion achieved a CR. At DL2, PBCAR0191 demonstrated durability of response in a patient with B-ALL for >11 months.

PBCAR0191, which incorporates Precision's N6 co-stimulatory domain, demonstrated a dose dependent increase in peak cell expansion at DL1, DL2 and DL3. Compared to standard lymphodepletion, enhanced lymphodepletion with PBCAR0191 at DL3 resulted in a ˜95-fold increase in peak cell expansion, and a ˜45-fold increase in area under the curve. This was associated with a higher complete response rate in NHL (75%) and a 83% ORR at day 28 or beyond.

### Safety & Tolerability:

In this dose escalation and dose expansion study, PBCAR0191 had an acceptable safety profile with no cases of GvHD, cases of Grade ≥3 CRS, or cases of Grade ≥3 ICANS. One patient experienced two concurrent infections (one Grade 2, the other Grade 3) attributable to lymphodepletion, which resolved prior to receiving cell administration. One NHL patient who was treated with PBCAR0191 and enhanced LD had previously received nine prior lines of therapy before entering the trial. The patient presented with persistent cytopenias at baseline and a history of infections, including bacterial sepsis. The patient had an episode of sepsis at day 27 which resolved. A partial response was achieved at day 34. Unfortunately, the patient became septic again and died at day 42 with grade 5 sepsis.

Patient demographics and interim data are summarized in Tables 1-5 below.

TABLE 1
Patient Demographics Indicate Advanced & Aggressive Disease

| | | NHL | | B-ALL | |
|---|---|---|---|---|---|
| | | (N = 16) | | (N=11) | |
| Age (y) | Median (min-max) | 63.5 | (34-81) | 57 | (26-72) |
| Subtype | Diffuse Large B-cell | 9 | (56%) | — | |
| | Mantle Cell | 5 | (31%) | — | |
| | High Grade B-cell | 2 | (13%) | — | |
| R/R | Refractory | 5 | (31%) | 4 | (37%) |
| | Relapsed | 11 | (69%) | 7 | (64%) |
| Stage | III/IV | 13 | (81%) | — | |
| Blast % | | n/a | | 47.5 | (5-94%) |
| Extranodal disease | | 10 | (63%) | — | |
| Prior lines | 4+ | 10 | (63%) | 9 | (82%) |

| | | | |
|---|---|---|---|
| Prior auto CAR T | 4 | (25%) | — |
| Prior auto-SCT | 3 | (19%) | — |

| | | | |
|---|---|---|---|
| Prior allo-SCT | — | 5 | (45%) |

| | | | | |
|---|---|---|---|---|
| Ki-67 | Median (min-max) | 70 | (10-100) | — |
| IPI | Median (min-max) | 3 | (1-4) | — |
| SPPD (mm²) | Median (min-max) | 2775 | (225-28947) | — |

TABLE 2
No Dose Limiting Toxicities Among 27 Patients Treated

| Phase 1 patients who received a dose of cells and | | Total | Total |
|---|---|---|---|
| have completed 28 day follow up by Nov. 16, 2020 | | NHL | B-ALL |
| (n = 27 patients) | | (n = 16) | (n = 11) |
| CRS | Grade 1 or | 9 (56%) | 5 (45%) |
| (Cytokine | Grade 2 | | |
| Release | Grade 3 or | 0 | 0 |
| Syndrome) | higher | | |
| ICANS | Grade 1 or | 5 (31%) | 2 (18%) |
| (Immune Effector | Grade 2 | | |
| Cell | Grade 3 or | 0 | 0 |
| Neurotoxicity) | higher | | |
| GvHD (Graft versus Host Disease) | | 0 | 0 |
| Infection | Grade 1 or | 1 (6%) | 0 (0%) |
| | Grade 2* | | |
| | Grade 3 or | 2 (13%) | 2 (11%) |
| | higher** | | |

Subsequent event post data cut-off - one NHL patient had a Grade 3 sepsis related to a previously known septic joint on day 27 which resolved on day 34. Sepsis occurred again at day 40 and patient died on day 42 with Grade 5 sepsis.
*One subject had Grade 1 Rhinovirus, unrelated
**1 NHL subject had Grade 3 sepsis - occurred prior to and resolved before cell administration
1 B-ALL subject had Grade 3 pneumonia - occurred prior to and resolved before cell administration (delayed cell infusion by 7 days)
1 B-ALL subject had Grade 3 pneumonia - occurred prior to and resolved before cell administration

TABLE 3
Summary of DL1 to DL3 Responses with Standard LD

| | | Standard Lymphodepletion* | | | | |
|---|---|---|---|---|---|---|
| | | NHL | | | B-ALL | |
| | | DL 1 | DL 2 | DL 3 | DL 2 | DL 3 |
| | | 3 × 10⁵ | 1 × 10⁶ | 3 × 10⁶ | 1 × 10⁶ | 3 × 10⁶ |
| | | cells/kg | cells/kg | cells/kg | cells/kg | cells/kg |
| | | (n = 3) | (n = 3) | (n = 6) | (n = 3) | (n = 4) |
| ORR at Day ≥28 | | 2 (67%) | 2 (67%) | 3 (50%) | 1 (33%) | 1 (25%) |
| Best | Complete | 0 | 1 (33%) | 2 (33%) | 1 (33%) | 1 (25%) |
| Response at | Response | | | | | |
| Day ≥28 | Partial | 2 (67%) | 1 (33%) | 1 (17%) | 0 | 0 |
| | Response | | | | | |
| | Progres- | 1 (33%) | 1 (33%) | 3 (50%) | 2 (67%) | 3 (75%) |
| | sive | | | | | |
| | Disease | | | | | |

*Fludarabine 30 mg/m²/day × 3 days + Cyclophosphamide 500 mg/m²/day × 3 days

TABLE 4
Acceptable Safety Profile Observed with Enhanced LD

| Phase 1 patients who received enhanced LD | | Total | Total |
|---|---|---|---|
| and completed 28-day follow up by Nov. 16, 2020 | | NHL | B-ALL |
| (n = 6 patients) | | (n = 4) | (n = 2) |
| CRS | Grade 1 or | 3 (75%) | 1 (50%) |
| (Cytokine | Grade 2 | | |
| Release | Grade 3 or | 0 | 0 |
| Syndrome ) | higher | | |
| ICANS | Grade 1 or | 1 (25%) | 0 (0%) |
| (Immune Effector | Grade 2 | | |
| Cell | Grade 3 or | 0 | 0 |
| Neurotoxicity) | higher | | |
| GvHD (Graft versus Host Disease) | | 0 | 0 |
| Infection | Grade 1 or | 0 (0%) | 0 (0%) |
| | Grade 2 | | |
| | Grade 3 or | 2 (50%) | 1 (50%) |
| | higher* | | |

Subsequent event post data cut-off - one NHL patient had a Grade 3 sepsis related to a previously known septic joint on day 27 which resolved on day 34. Sepsis occurred again at day 40 and patient died on day 42 with Grade 5 sepsis.
*1 NHL subject had grade 3 sepsis - occurred prior to and resolved before cell administration
1 B-ALL subject had grade 3 pneumonia - occurred prior to and resolved before cell administration (delayed cell infusion by 7 days)

TABLE 5
NHL: Summary of Responses - DL3 with Enhanced LD^{1, 3}

| | | Enhanced LD | | |
|---|---|---|---|---|
| | | NHL | B-ALL | Total |
| | | (n=4) | (n=2) | (n=6) |
| ORR at Day ≥28 | | 4 (100%) | 1 (50%) | 5 (83%) |
| Best Response | Complete | 3 (75%) | 1 (50%) | 4 (67%) |
| at Day ≥28 | Response | | | |
| | Partial | 1 (25%) | 0 | 1 (17%) |
| | Response | | | |
| | Progressive | 0 | 1 (50%)² | 1 (17%) |
| | Disease | | | |

¹Enhanced LD: fludarabine 30 mg/m²/day × 4 days + cyclophosphamide 1000 mg/m²/day × 3 days
²Patient was given CAR T cells 7 days late due to an infection
³1 NHL patient received a second infusion of cells at Day 10 without repeat LD

### Example 3

### Allogeneic CD20 CAR T Therapy in Non-Hodgkin Lymphoma, Chronic Lymphoblastic Leukemia, or Small Lymphocytic Lymphoma

### A. Background

PBCAR20A is an off-the-shelf allogeneic CD19-targeted chimeric antigen receptor (CAR) T cell product derived from qualified donor T cells that have been genetically edited to remove the expression of the endogenous T cell receptor (TCR) and insert the CAR in the same locus.

This is a multicenter, nonrandomized, open-label, parallel assignment, single-dose, dose-escalation, and dose-expansion study to evaluate the safety and tolerability, find an appropriate dose to optimize safety and efficacy, and evaluate clinical activity of PBCAR20A in subjects with relapsed/refractory (r/r) Non-Hodgkin Lymphoma (NEIL) or r/r Chronic Lymphocytic Leukemia (CLL) or Small Lymphocytic Lymphoma (SLL). Before initiating PBCAR20A, subjects will be administered lymphodepletion chemotherapy composed of fludarabine and cyclophosphamide. At Day 0 of the Treatment Period, subjects will receive a single intravenous (IV) infusion of PBCAR20A. All subjects are monitored during the treatment period through Day 28. All subjects who receive a dose of PBCAR20A will be followed in a separate long-term follow-up (LTFU) study for 15 years after exiting this study.

In this study, PBCAR20A, allogeneic anti-CD20 CAR T Cells, is used to treat patients with relapsed or refractory (r/r) Non-Hodgkin Lymphoma (NHL) or r/r Chronic Lymphocytic Leukemia (CLL) or Small Lymphocytic Lymphoma (SLL). Lymphodepletion will be conducted several days prior to PBCAR20A infusion. A combination of fludarabine and cyclophosphamide will be used for lymphodepletion. A single dose of PBCAR20A cells will be infused, and a classic “3+3” dose escalation will be applied.

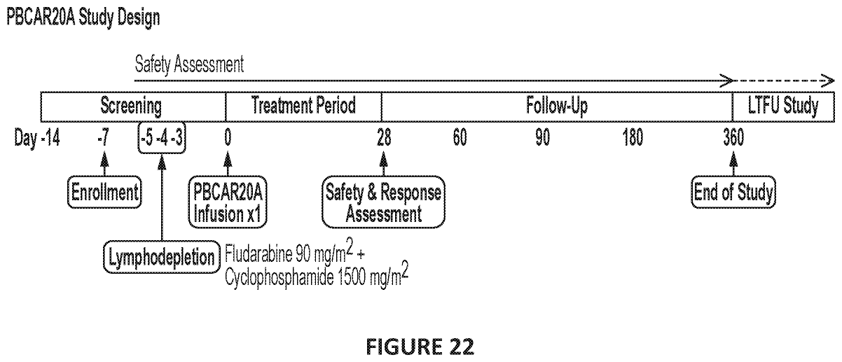

The lymphodepletion regimen includes the administration of cyclophosphamide (500 mg/m²) and fludarabine (30 mg/m²) on days −5 to −3 prior to PBCAR20A infusion. The PBCAR20A study design is illustrated in FIG. 22.

Primary outcome measures include:
Maximum Tolerated Dose (MTh) [Time Frame: Day 1-Day 28]
To determine the maximum tolerated dose (MTD), which is defined as the dose level at which fewer than 33% of patients experience a dose limiting toxicity (DLT) using a 3+3 strategy.
Number of Participants with Dose Limiting Toxicity(ies) [Time Frame: 1 year]
To assess adverse events as dose limiting toxicities as defined by the protocol and CTCAE v5.0.
   Secondary outcome measures include:
Objective Response Rate of Patients [Time Frame: 1 year]
To assess clinical activity as response in B-ALL by the NCCN Guidelines on ALL (NCCN, 2017) and in NHL by the revised Lugano Classification (Cheson et al, 2016), both reported as objective response rate.
   Other outcome measures include:
Area Under the Curve [AUC] [Time Frame: 1 year]
To evaluate Area Under the Curve [AUC] of PBCAR20A in patients tested.
   Ages Eligible for Study: 18 Years and older (Adult, Older Adult)

### Sexes Eligible for Study: All

### Accepts Healthy Volunteers: No

### Key Inclusion Criteria

### Criteria for NHL:

r/r B-cell NHL that is histologically confirmed by archived tumor biopsy tissue from the last relapse and corresponding pathology report.
Measurable or detectable disease according to the Lugano classification.
Primary refractory disease or r/r disease after a response to 2 prior regimens.

### Criteria for CLL/SLL:

Diagnosis of CLL with indication for treatment based on the iwCLL guidelines and clinically measurable disease or SLL with measurable disease that is biopsy-proven SLL.
Previously failed/tolerant to at least 2 prior lines of systemic targeted therapy of known benefit.
   Criteria for both NHL and CLL/SLL:
Study participant has an Eastern Cooperative Oncology Group (ECOG) Performance Status score of 0 or 1.
Study participant has adequate bone marrow, renal, hepatic, pulmonary, and cardiac function.

### Key Exclusion Criteria

### Criteria for NHL:

Requirement for urgent therapy due to mass effects such as bowel obstruction, spinal cord, or blood vessel compression.
Active central nervous system (CNS) disease. A negative computed tomography (CT)/magnetic resonance imaging (MRI) is required at Screening if the study participant has a history of CNS lymphoma.

### Criteria for CLL/SLL:

Active CNS disease. A negative lumbar puncture is required at Screening if the study participant has a history of CNS disease.

### Criteria for NHL and CLL/SLL:

Previous malignancy, besides the malignancies of inclusion (B-cell NHL or CLL/SLL), that in the investigator's opinion, has a high risk of relapse in the next 2 years.
Active uncontrolled fungal, bacterial, viral, protozoal, or other infection.
Any form of primary immunodeficiency.
History of human immunodeficiency virus (HIV) infection.
Active hepatitis B or C.
Uncontrolled cardiovascular disease.
Hypertension crisis or hypertensive encephalopathy within 3 months prior to Screening.
Presence of a CNS disorder that renders ineligible for treatment.
History of a genetic syndrome such as Fanconi anemia, Kostmann syndrome, Shwachman Diamond syndrome, or any other known bone marrow failure syndrome.
Active hemolytic anemia.
Received ASCT within 45 days of Screening if the study participant has met the rest of the count requirements.
Must not have received systemic corticosteroid therapy for at least 1 day prior to initiating lymphodepletion chemotherapy.
Received a systemic biologic agent within 30 days or 5 half-lives.
Received a live vaccine within 4 weeks before Screening.
Received standard cytotoxic chemotherapy within 10 days of Screening.
Radiotherapy within 4 weeks determined on a case-by-case basis.
Presence of a pleural/peritoneal/pericardial catheter.
Current use of any anticoagulant or antiplatelet therapy.
Additional criteria apply

### Example 4

### Allogeneic CD269 (BCMA) CAR T Therapy in Multiple Myeloma

### A. Background

PBCAR269A is an off-the-shelf allogeneic CD269 (BCMA)-targeted chimeric antigen receptor (CAR) T cell product derived from qualified donor T cells that have been genetically edited to remove the expression of the endogenous T cell receptor (TCR) and insert the CAR in the same locus.

This is a multicenter, nonrandomized, open-label, parallel assignment, single-dose, dose-escalation, and dose-expansion study to evaluate the safety and clinical activity of PBCAR269A in adults with r/r multiple myeloma. Before initiating the study treatment PBCAR269A, all study participants will be administered lymphodepletion chemotherapy. The initial lymphodepletion chemotherapy regimen will be composed of fludarabine and cyclophosphamide during the Screening Period. On Day 0 of the Treatment Period, study participants will receive a single intravenous (N) infusion of PBCAR269A. All subjects are monitored during the treatment period through Day 28. All subjects who receive a dose of PBCAR269A will be followed in a separate long-term follow-up (LTFU) study for 15 years after exiting this study.

In Phase I, 3 escalating dose groups will be enrolled and treated sequentially, with the possibility of a single de-escalation. Within each dose group, at least 3 and at most 6 study participants will be treated with a single dose of PBCAR269A using a standard 3+3 design. The starting dose of PBCAR269A will be 6×10⁵ CAR T cells/kg body weight. Subsequent dose groups will be treated with escalating doses to a maximum dose of 6×10⁶ CAR T cells/kg. In the absence of DLTs, the dose will be increased using a fixed-dose scheme.

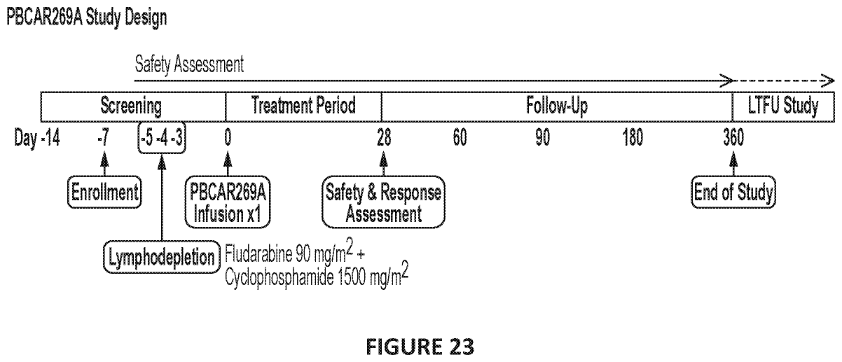

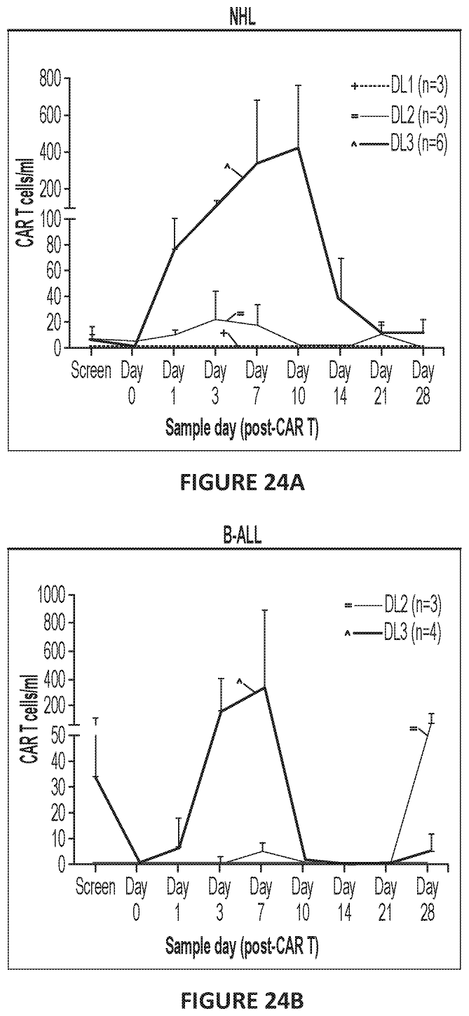

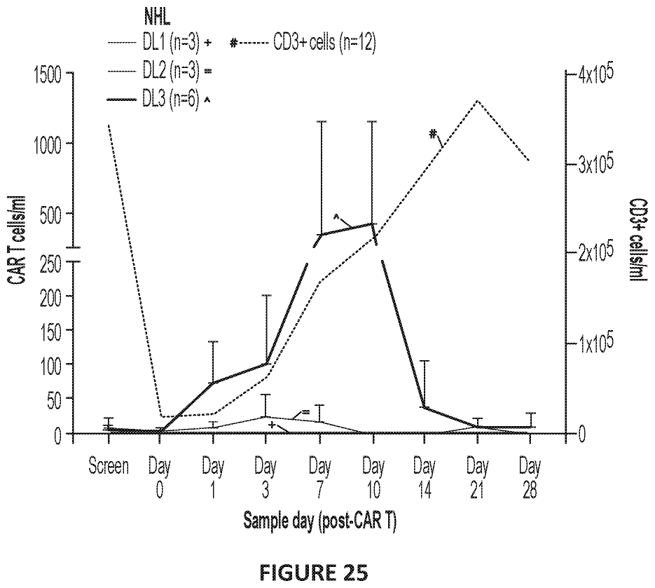

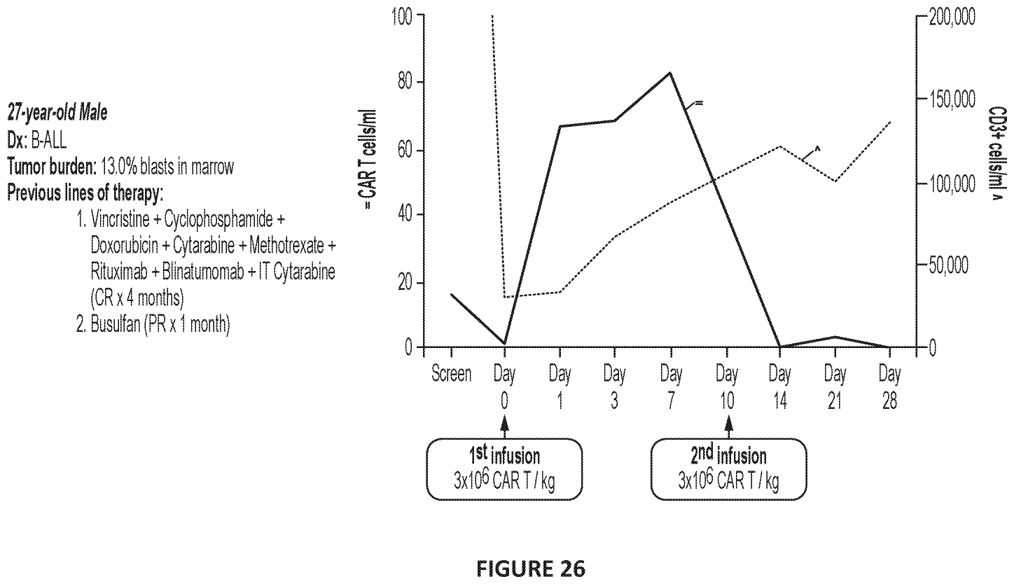

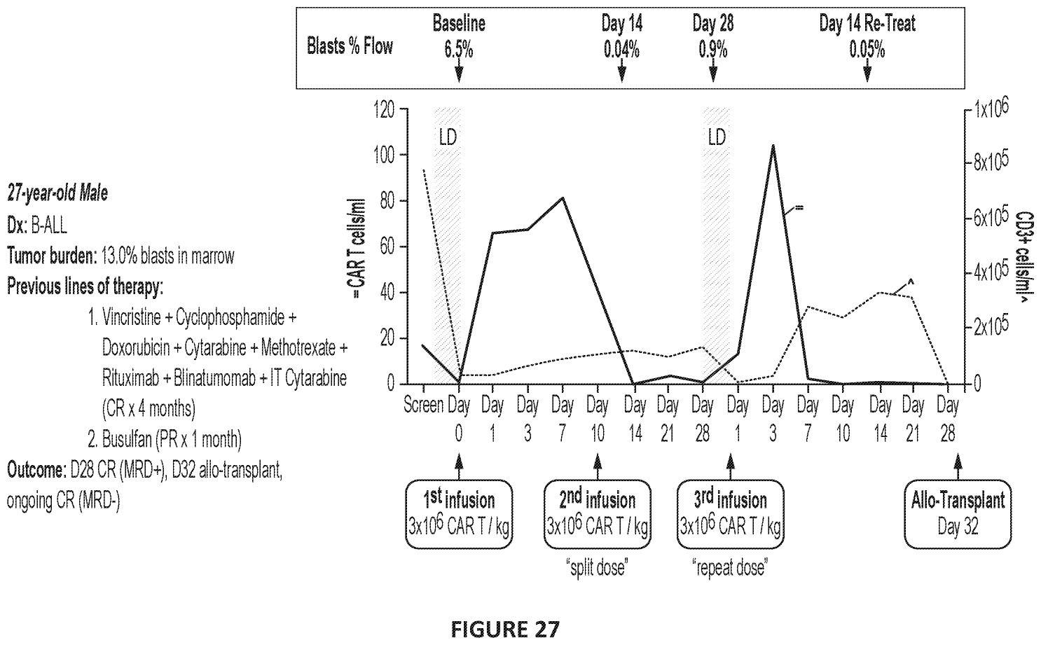

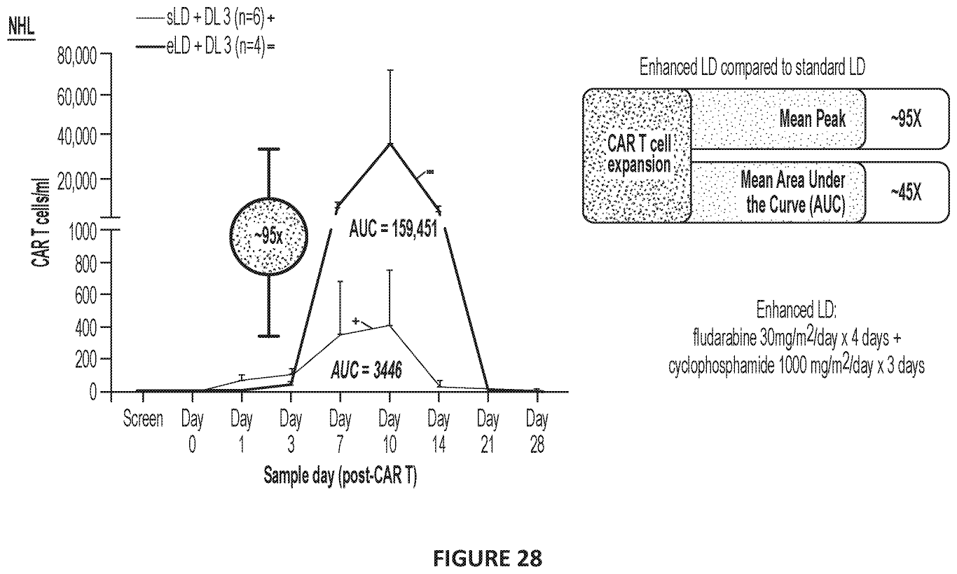

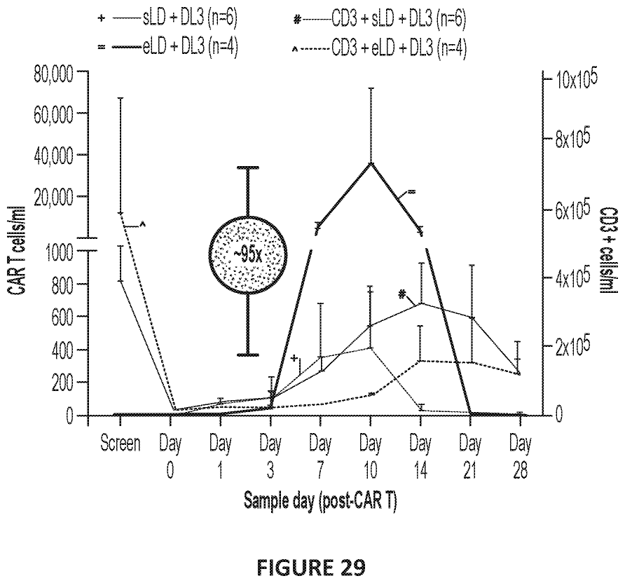

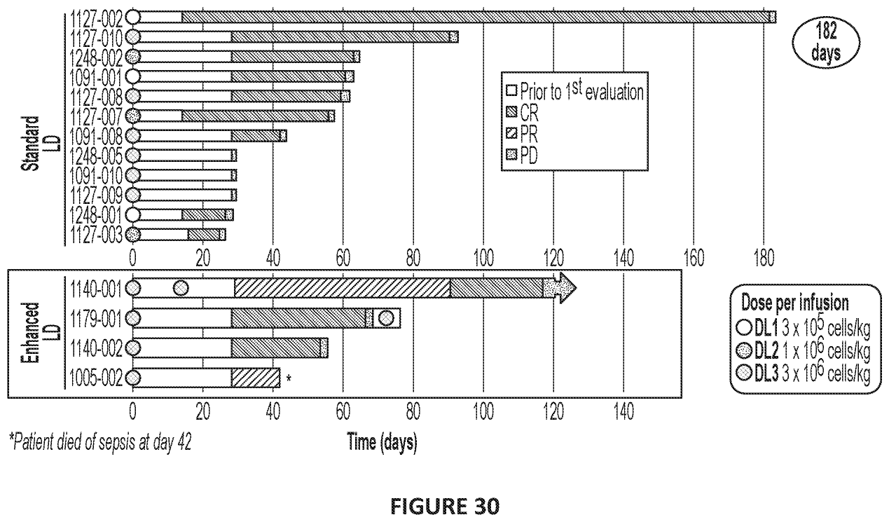

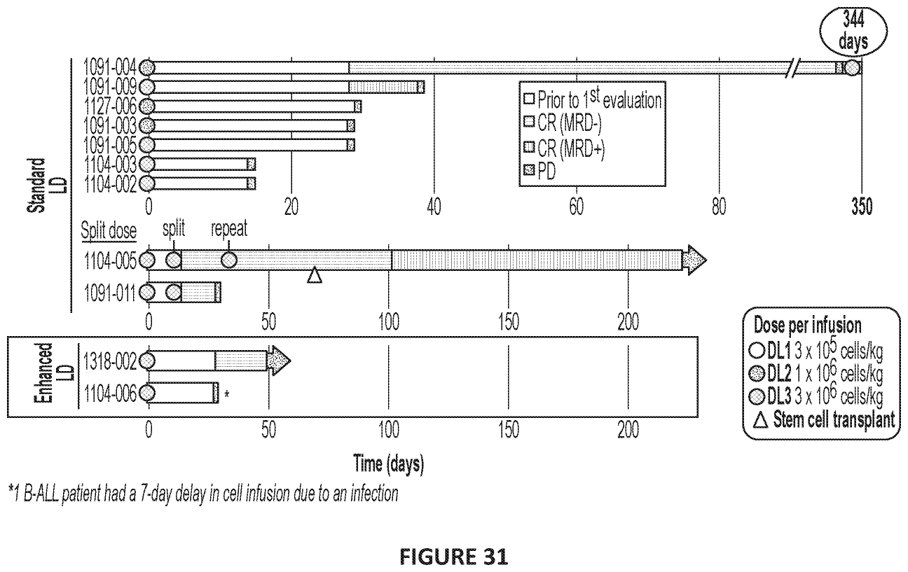

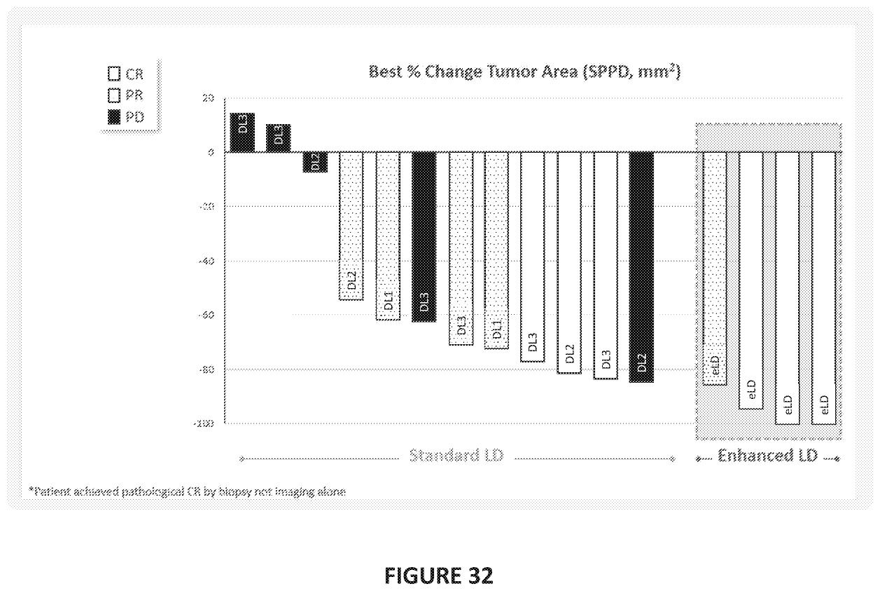

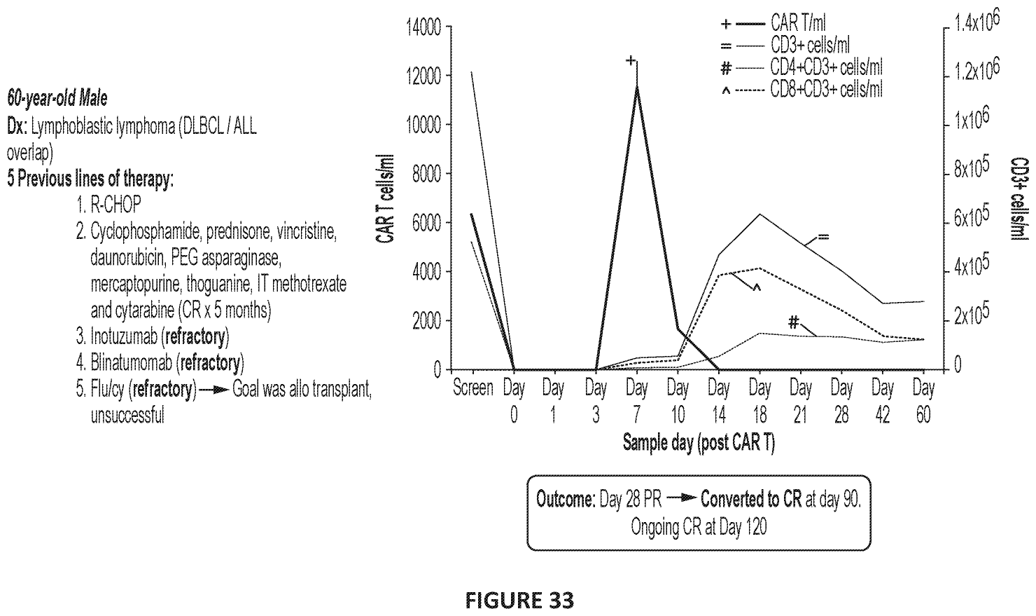

The lymphodepletion regimen includes the administration of cyclophosphamide (500 mg/m²) and fludarabine (30 mg/m²) on days −5 to −3 prior to PBCAR269A infusion. The PBCAR269A study design is illustrated in FIG. 23.

Primary outcome measures include:
Maximum Tolerated Dose (MTD) of PBCAR269A [Time Frame: Day 1-Day 28]
To determine the maximum tolerated dose (MTD), which is defined as the dose level at which fewer than 33% of patients experience a dose limiting toxicity (DLT) using a 3+3 strategy.
   Secondary outcome measures include:
Number of Participants with Dose Limiting Toxicity(ies) [Time Frame: 1 year]
To assess adverse events as dose limiting toxicities as defined by the protocol and CTCAE v5.0.
Objective Response Rate of Patients [Time Frame: 1 year]
To assess ORR to treatment with PBCAR269A through Day 360 will be noted using the IMWG response criteria.
The ORR is defined as the proportion of study participants meeting the definition of response within the study population to the response evaluable population. ORR will be summarized by number and percentage of study participants meeting the definition of ORR along with the corresponding exact 95% CIs. DoR, defined as the duration (days) from initial response to disease relapse, progression, or death will be descriptively analyzed using Kaplan-Meier methods. The number of study participants achieving DoR at 3, 6, 9, and 12 months will also be calculated. Exploratory efficacy analyses include changes from Baseline in CBC counts, CAR T cells, cytokines, and CRP levels.
   Ages eligible for study: 18 Years and older (Adult, Older Adult)
   Sexes eligible for study: All
   Accepts healthy volunteers: No

### Inclusion Criteria:

Diagnosis of MM with relapsed and/or refractory disease according to the IMWG criteria.
Measurable disease at Screening including at least 1 of the criteria below. Note: Study participants with immunoglobulin (Ig) A myeloma in whom serum protein electrophoresis is deemed unreliable due to emigration of normal serum proteins with the paraprotein in the beta region may be considered eligible provided total serum IgA level is >400 mg/dL.
Serum myeloma (M)-protein ≥0.5 g/dL or, urine M-protein >200 mg/24 hour.
Serum free light chain >10 mg/dL with abnormal kappa:lambda ratio.
Imaging consistent plasmacytoma and the presence of any clonal plasma cells in peripheral blood or bone marrow.
Study participants must be refractory to 2 prior MM treatment regimens including an immunomodulatory imide drug and a protease inhibitor prior to entering the study. Study participants must have recovered or stabilized to Grade ≤2 from any AEs experienced during prior treatment with the exception of neuropathy. Prior therapy requirements are as follows:
Undergone ≥1 complete cycle of treatment for each regimen, unless progressive disease was the best response to the regimen.
Must have received an immunomodulatory agent, a proteasome inhibitor, and an anti-CD38 antibody.
Study participants who are not candidates for ≥1 of the above treatments may still be considered eligible.
Has an Eastern Cooperative Oncology Group (ECOG) Performance Status score of 0 or 1.
Has adequate bone marrow, renal, hepatic, pulmonary, and cardiac function defined as:
Estimated glomerular filtration rate >60 mL/min/1.73 m². A 24-hour urine collection for creatinine clearance may be used at the investigator's discretion.
Alanine aminotransferase (ALT) and aspartate aminotransferase (AST) levels both
≤3 times of upper limit of normal, unless there is suspected disease in the liver, in which case, no limit is set provided serum bilirubin is within eligibility criterion.
Total bilirubin <2.0 mg/dL, except in study participants with Gilbert's syndrome.
Platelet count >50,000/μL (platelet transfusions acceptable); neutrophils >750/μL.
Left ventricular ejection fraction (LVEF)>45% as assessed by echocardiogram (ECHO) or multiple gated acquisition (MUGA) scan performed within 1 month before starting lymphodepleting chemotherapy. ECHO results performed within 6 months before Screening and at least 28 days after the last cancer treatment may be acceptable if the study participant has not received any treatment with cardiotoxicity risks.
No clinically significant evidence of pericardial effusion or pleural effusion based on investigator's opinion.
Baseline oxygen saturation >92% on room air.
Pulmonary function tests including forced expiratory volume at 1 sec, forced vital capacity, total lung capacity, diffusion capacity of lung for carbon monoxide ≥50% of predicted values. Study participant characteristics
All study participants must be willing to practice birth control and refrain from donating sperm or oocytes from the time of enrollment in this study through 3 months after receiving the study treatment.
Women of childbearing potential (WOCBP) must test negative for pregnancy at Screening because of the potentially harmful effects of the preparative chemotherapy to the fetus. WOCBP are defined as any women who are not postmenopausal or who have not had a hysterectomy. Postmenopausal is defined as women over the age of 55 who have not had a menstrual period for ≥1 year.
Capable of giving signed informed consent.

### Exclusion Criteria:

Study participant has clinically significant organ involvement by amyloid protein.
Study participant has plasma cell leukemia, Waldenstrom's macroglobulinemia, or POEMS syndrome.
History of class III or IV congestive heart failure or severe non-ischemic cardiomyopathy, unstable or poorly controlled angina, myocardial infarction, or ventricular arrhythmia within the previous 6 months of starting study treatment.
History or presence of clinically relevant central nervous system (CNS) pathology.
Active uncontrolled fungal, bacterial, viral, protozoal, or other infection.
Any form of primary immunodeficiency (e.g., severe combined immunodeficiency disease).
History of human immunodeficiency virus (HIV) infection.
Active hepatitis B or hepatitis C confirmed by polymerase chain reaction (PCR). Study participant positive for inactive hepatitis B will be allowed to enroll if on prophylactic treatment.
History of hypertension crisis or hypertensive encephalopathy within 3 months prior to Screening.
History of severe immediate hypersensitivity reaction to any of the agents used in this study.
Abnormal findings during the Screening Period or any other medical condition(s) or laboratory findings that, in the opinion of the investigator, might jeopardize the study participant's safety.
History of genetic syndrome such as Fanconi anemia, Kostmann syndrome, Shwachman-Diamond syndrome, or any other known bone marrow failure syndrome.
Study participants with active hemolytic anemia.
Study participant has received autologous stem cell transplant within 12 weeks of Screening or an allogeneic stem cell transplant within 6 months of starting study treatment. Study participants who have received an allogeneic transplant must be off all immunosuppressive medications for 6 weeks without signs of GvHD.
Study participants with second malignancies in addition to MM if the second malignancy has required treatment within the last 3 years or is not in complete remission, with the exception of non-metastatic basal cell or squamous cell skin carcinoma.
Study participant has received systemic biologic agent within 28 days. Participation in non-interventional registries or epidemiological studies is not excluded.
Study participant has received live vaccine within 4 weeks before Screening. Non-live virus vaccines are not excluded.
Before initiation of lymphodepletion, study participants must have recovered or stabilized to Grade ≤2 from any AEs experienced during prior treatment with the exception of neuropathy.
Radiotherapy within 4 weeks before Screening should be discussed with the monitor.
Presence of pleural/peritoneal/pericardial catheter.
Current use of any anticoagulant or antiplatelet therapy.

Sequence Listing

SEQ ID NO: 1
TGGCCTGGAGCAACAAATCTGA
SEQ ID NO: 2
ACCGGACCTCGTTGTTTAGACT
SEQ ID NO: 3
EVKLQESGPGLVAPSQSLSVTCTVSGVSLPDYGVSWIRQPPRKGLEWLGVIWGSETTYY
NSALKSRLTIIKDNSKSQVILKMNSLQTDDTAIYYCAKHYYYGGSYAMDYWGQGTSVT
VSS
SEQ ID NO: 4
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEIT
SEQ ID NO: 5
MALPVTALLLPLALLLHAARPDIQMTQTTSSLSASLGDRVTISCRASQDISKYLNWYQQ
KPDGTVKLLIYHTSRLHSGVPSRFSGSGSGTDYSLTISNLEQEDIATYFCQQGNTLPYTFG
GGTKLEITGGGGSGGGGSGGGGSEVKLQESGPGLVAPSQSLSVTCTVSGVSLPDYGVSW
IRQPPRKGLEWLGVIWGSETTYYNSALKSRLTIIKDNSKSQVFLKMNSLQTDDTAIYYCA
KHYYYGGSYAMDYWGQGTSVTVSSTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPR
SEQ ID NO: 6
EVQLVQSGAEVKKPGESLKISCKGSGYSFTSYWIGWVRQMPGKGLEWMGIIYPGDSDT
RYSPSFQGQVTISADKSITTAYLQWSSLKASDTAMYYCARHPSYGSGSPNFDYWGQGTL
VTVSS
SEQ ID NO: 7
DIVMTQTPLSSPVTLGQPASISCRSSQSLVYSDGNTYLSWLQQRPGQPPRLLIYKISNRFS
GVPDRFSGSGAGTDFTLKISRVEAEDVGVYYCVQATQFPLTFGGGTKVEIK
SEQ ID NO: 8
MALPVTALLLPLALLLHAARPDIVMTQTPLSSPVTLGQPASISCRSSQSLVYSDGNTYLS
WLQQRPGQPPRLLIYKISNRFSGVPDRFSGSGAGTDFTLKISRVEAEDVGVYYCVQATQF
PLTFGGGTKVEIKGGGGSGGGGSGGGGSEVQLVQSGAEVKKPGESLKISCKGSGYSFTS
YWIGWVRQMPGKGLEWMGIIYPGDSDTRYSPSFQGQVTISADKSITTAYLQWSSLKASD
TAMYYCARHPSYGSGSPNFDYWGQGTLVTVSSTTTPAPRPPTPAPTIASQPLSLRPEACR
PAAGGAVHTRGLDFACDIYIWAPLAGTCGVLLLSLVITLYCKASRKKAAAAAKSPFASP
ASSAQEEDASSCRAPSEEEGSCELRVKFSRSADAPAYQQGQNQLYNELNLGRREEYDVL
DKRRGRDPEMGGKPRRKNPQEGLYNELQKDKMAEAYSEIGMKGERRRGKGHDGLYQ
GLSTATKDTYDALHMQALPPR
SEQ ID NO: 9
QEED
SEQ ID NO: 10
EEEE
SEQ ID NO: 11
SEEE
SEQ ID NO: 12
KASRKKAAAAAKSPFASPASSAQEEDASSCRAPSEEEGSCEL
SEQ ID NO: 13
KRGRKKLLYIFKQPFMRPVQTTQEEDGCSCRFPEEEEGGCEL
SEQ ID NO: 14
TTTPAPRPPTPAPTIASQPLSLRP
SEQ ID NO: 15
EACRPAAGGAVHTRGLDFACDIYIWAPLAGTCGVLLLSLVITLYC
SEQ ID NO: 16
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 17
MALPVTALLLPLALLLHAARP
SEO ID NO: 18
GGGCGGAGTTAGGGCGGAGCCAATCAGCGTGCGCCGTTCCGAAAGTTGCCTTTTATG
GCTGGGCGGAGAATGGGCGGTGAACGCCGATGATTATATAAGGACGCGCCGGGTGT
GGCACAGCTAGTTCCGTCGCAGCCGGGATTTGGGTCGCGGTTCTTGTTTGT
SEQ ID NO: 19
MNTKYNKEFLLYLAGFVDGDGSIYAVIYPHQRAKFKHFLKLLFTVSQSTKRRWFLDKL
VDEIGVGYVYDLPRTSEYRLSEIKPLHNFLTQLQPFLKLKQKQANLVLKIIEQLPSAKESP
DKFLEVCTWVDQIAALNDSRTRKTTSETVRAVLDSLPGSVGGLSPSQASSAASSASSSPG
SGISEALRAGAGSGTGYNKEFLLYLAGFVDGDGSIYACIRPRQGSKFKHRLTLGFAVGQ
KTQRRWFLDKLVDEIGVGYVYDRGSVSEYVLSEIKPLHNFLTQLQPFLKLKQKQANLVL
KIIEQLPSAKESPDKFLEVCTWVDQIAALNDSKTRKTTSETVRAVLDSLSEKKKSSP
SEP ID NO: 20
MNTKYNKEFLLYLAGFVDGDGSIIAQIKPNQSYKFKHQLSLAFQVTQKTQRRWFLDKLV
DEIGVGYVRDRGSVSDYILSEIKPLHNFLTQLQPFLKLKQKQANLVLKIIWRLPSAKESPD
KFLEVCTWVDQIAALNDSKTRKTTSETVRAVLDSLSEKKKSSP
SEQ ID NO: 21
LAGLIDADG
SEQ ID NO: 22
ATATCCAGAACCCTGACCCTGCCGTGTACCAGCTGAGAGACTCTAAATCCAGTGACA
AGTCTGTCTGCCTATTCACCGATTTTGATTCTCAAACAAATGTGTCACAAAGTAAGG
ATTCTGATGTGTATATCACAGACAAAACTGTGCTAGACATGAGGTCTATGGACTTCA
AGAGCAACAGTGCTGTGGCCTGGAGCAACAAATCTGACTTTGCATGTGCAAACGCC
TTCAACAACAGCATTATTCCAGAAGACACCTTCTTCCCCAGCCCAGGTAAGGGCAGC
TTTGGTGCCTTCGCAGGCTGTTTCCTTGCTTCAGGAATGGCCAGGTTCTGCCCAGAGC
TCTGGTCAATGATGTCTAAAACTCCTCTGATTGGTGGTCTCGGCCTTATCCATTGCCA
CCAAAACCCTCTTTTTACTAAGAAACAGTGAGCCTTGTTCTGGCAGTCCAGAGAATG
ACACGGGAAAAAAGCAGATGAAGAGAAGGTGGCAGGAGAGGGCACGTGGCCCAGC
CTCAGTCTCTCCAACTGAGTTCCTGCCTGCCTGCCTTTGCTCAGACTGTTTGCCCCTT
ACTGCTCTTCTAGGCCTCATTCTAAGCCCCTTCTCCAAGTTGCCTCTCCTTATTTCTCC
CTGTCTGCCAAAAAATCTTTCCCAGCTCACTAAGTCAGTCTCACGCAGTCACTCATT
AACCCACCAATCACTGATTGTGCCGGCACATGAATGCACCAGGTGTTGAAGTGGAG
GAATTAAAAAGTCAGATGAGGGGTGTGCCCAGAGGAAGCACCATTCTAGTTGGGGG
AGCCCATCTGTCAGCTGGGAAAAGTCCAAATAACTTCAGATTGGAATGTGTTTTAAC
TCAGGGTTGAGAAAACAGCTACCTTCAGGACAAAAGTCAGGGAAGGGCTCTCTGAA
GAAATGCTACTTGAAGATACCAGCCCTACCAAGGGCAGGGAGAGGACCCTATAGAG
GCCTGGGACAGGAGCTCAATGAGAAAGGAGAAGAGCAGCAGGCATGAGTTGAATG
AAGGAGGCAGGGCCGGGTCACAGGGCCTTCTAGGCCATGAGAGGGTAGACAGTATT
CTAAGGACGCCAGAAAGCTGTTGATCGGCTTCAAGCAGGGGAGGGACACCTAATTT
GCTTTTCTTTTTTTTTTTTTTTTTTTTTTTTTTTTTTGAGATGGAGTTTTGCTCTTGTTGC
CCAGGCTGGAGTGCAATGGTGCATCTTGGCTCACTGCAACCTCCGCCTCCCAGGTTC
AAGTGATTCTCCTGCCTCAGCCTCCCGAGTAGCTGAGATTACAGGCACCCGCCACCA
TGCCTGGCTAATTTTTTGTATTTTTAGTAGAGACAGGGTTTCACTATGTTGGCCAGGC
TGGTCTCGAACTCCTGACCTCAGGTGATCCACCCGCTTCAGCCTCCCAAAGTGCTGG
GATTACAGGCGTGAGCCACCACACCCGGCCTGCTTTTCTTAAAGATCAATCTGAGTG
CTGTACGGAGAGTGGGITGTAAGCCAAGAGTAGAAGCAGAAAGGGAGCAGTTGCA
GCAGAGAGATGATGGAGGCCTGGGCAGGGTGGTGGCAGGGAGGTAACCAACACCA
TTCAGGTTTCAAAGGTAGAACCATGCAGGGATGAGAAAGCAAAGAGGGGATCAAG
GAAGGCAGCTGGATTTTGGCCTGAGCAGCTGAGTCAATGATAGTGCCGTTTACTAAG
AAGAAACCAAGGAAAAAATTTGGGGTGCAGGGATCAAAACTTTTTGGAACATATGA
AAGTACGTGTTTATACTCTTTATGGCCCTTGTCACTATGTATGCCTCGCTGCCTCCAT
TGGACTCTAGAATGAAGCCAGGCAAGAGCAGGGTCTATGTGTGATGGCACATGTGG
CCAGGGTCATGCAACATGTACTTTGTACAAACAGTGTATATTGAGTAAATAGAAATG
GTGTCCAGGAGCCGAGGTATCGGTCCTGCCAGGGCCAGGGGCTCTCCCTAGCAGGT
GCTCATATGCTGTAAGTTCCCTCCAGATCTCTCCACAAGGAGGCATGGAAAGGCTGT
AGTTGTTCACCTGCCCAAGAACTAGGAGGTCTGGGGTGGGAGAGTCAGCCTGCTCTG
GATGCTGAAAGAATGTCTGTTTTTCCTTTTAGAAAGTTCCTGTGATGTCAAGCTGGTC
GAGAAAAGCTTTGAAACAGGTAAGACAGGGGTCTAGCCTGGGTTTGCACAGGATTG
CGGAAGTGATGAACCCGCAATAACCCTGCCTGGATGAGGGAGTGGGAAGAAATTAG
TAGATGTGGGAATGAATGATGAGGAATGGAAACAGCGGTTCAAGACCTGCCCAGAG
CTGGGTGGGGTCTCTCCTGAATCCCTCTCACCATCTCTGACTTTCCATTCTAAGCACT
TTGAGGATGAGTTTCTAGCTTCAATAGACCAAGGACTCTCTCCTAGGCCTCTGTATTC
CTTTCAACAGCTCCACTGTCAAGAGAGCCAGAGAGAGCTTCTGGGTGGCCCAGCTGT
GAAATTTCTGAGTCCCTTAGGGATAGCCCTAAACGAACCAGATCATCCTGAGGACA
GCCAAGAGGTTTTGCCTTCTTTCAAGACAAGCAACAGTACTCACATAGGCTGTGGGC
AATGGTCCTGTCTCTCAAGAATCCCCTGCCACTCCTCACACCCACCCTGGGCCCATA
TTCATTTCCATTTGAGTTGTTCTTATTGAGTCATCCTTCCTGTGGTAGCGGAACTCAC
TAAGGGGCCCATCTGGACCCGAGGTATTGTGATGATAAATTCTGAGCACCTACCCCA
TCCCCAGAAGGGCTCAGAAATAAAATAAGAGCCAAGTCTAGTCGGTGTTTCCTGTCT
TGAAACACAATACTGTTGGCCCTGGAAGAATGCACAGAATCTGTTTGTAAGGGGAT
ATGCACAGAAGCTGCAAGGGACAGGAGGTGCAGGAGCTGCAGGCCTCCCCCACCCA
GCCTGCTCTGCCTTGGGGAAAACCGTGGGTGTGTCCTGCAGGCCATGCAGGCCTGGG
ACATGCAAGCCCATAACCGCTGTGGCCTCTTGGTTTTACAGATACGAACCTAAACTT
TCAAAACCTGTCAGTGATTGGGTTCCGAATCCTCCTCCTGAAAGTGGCCGGGTTTAA
TCTGCTCATGACGCTGCGGCTGTGGTCCAGCTGAGGTGAGGGGCCTTGAAGCTGGGA
GTGGGGTTTAGGGACGCGGGTCTCTGGGTGCATCCTAAGCTCTGAGAGCAAACCTCC
CTGCAGGGTCTTGCTTTTAAGTCCAAAGCCTGAGCCCACCAAACTCTCCTACTTCTTC
CTGTTACAAATTCCTCTTGTGCAATAATAATGGCCTGAAACGCTGTAAAATATCCTC
ATTTCAGCCGCCTCAGTTGCACTTCTCCCCTATGAGGTAGGAAGAACAGTTGTTTAG
AAACGAAGAAACTGAGGCCCCACAGCTAATGAGTGGAGGAAGAGAGACACTTGTGT
ACACCACATGCCTTGTGTTGTACTTCTCTCACCGTGTAACCTCCTCATGTCCTCTCTC
CCCAGTACGGCTCTCTTAGCTCAGTAGAAAGAAGACATTACACTCATATTACACCCC
AATCCTGGCTAGAGTCTCCGCACCCTCCTCCCCCAGGGTCCCCAGTCGTCTTGCTGA
CAACTGCATCCTGTTCCATCACCATCAAAAAAAAACTCCAGGCTGGGTGCGGGGGC
TCACACCTGTAATCCCAGCACTTTGGGAGGCAGAGGCAGGAGGAGCACAGGAGCTG
GAGACCAGCCTGGGCAACACAGGGAGACCCCGCCTCTACAAAAAGTGAAAAAATTA
ACCAGGTGTGGTGCTGCACACCTGTAGTCCCAGCTACTTAAGAGGCTGAGATGGGA
GGATCGCTTGAGCCCTGGAATGTTGAGGCTACAATGAGCTGTGATTGCGTCACTGCA
CTCCAGCCTGGAAGACAAAGCAAGATCCTGTCTCAAATAATAAAAAAAATAAGAAC
TCCAGGGTACATTTGCTCCTAGAACTCTACCACATAGCCCCAAACAGAGCCATCACC
ATCACATCCCTAACAGTCCTGGGTCTTCCTCAGTGTCCAGCCTGACTTCTGTTCTTCC
TCATTCCAGATCTGCAAGATTGTAAGACAGCCTGTGCTCCCTCGCTCCTTCCTCTGCA
TTGCCCCTCTTCTCCCTCTCCAAACAGAGGGAACTCTCCTACCCCCAAGGAGGTGAA
AGCTGCTACCACCTCTGTGCCCCCCCGGCAATGCCACCAACTGGATCCTACCCGAAT
TTATGATTAAGATTGCTGAAGAGCTGCCAAACACTGCTGCCACCCCCTCTGTTCCCTT
ATTGCTGCTTGTCACTGCCTGACATTCACGGCAGAGGCAAGGCTGCTGCAGCCTCCC
CTGGCTGTGCACATTCCCTCCTGCTCCCCAGAGACTGCCTCCGCCATCCCACAGATG
ATGGATCTTCAGTGGGTTCTCTTGGGCTCTAGGTCCTGCAGAATGTTGTGAGGGGTT
TATTTTTTTTTAATAGTGTTCATAAAGAAATACATAGTATTCTTCTTCTCAAGACGTG
GGGGGAAATTATCTCATTATCGAGGCCCTGCTATGCTGTGTATCTGGGCGTGTTGTA
TGTCCTGCTGCCGATGCCTTC
SEQ ID NO: 23
PNIQNPDPAVYQLRDSKSSDKSVCLFTDFDSQTNVSQSKDSDVYITDKTVLDMRSMDFK
SNSAVAWSNKSDFACANAFNNSIIPEDTFFPSPESSCDVKLVEKSFETDTNLNFQNLSVIG
FRILLLKVAGFNLLMTLRLWSS

1. A method of immunotherapy for treating cancer in a subject, said method comprising:
(a) administering to said subject a lymphodepletion regimen that includes no greater than a minimal effective dose of any biological lymphodepletion agent, wherein said lymphodepletion regimen comprises one or more chemotherapeutic lymphodepletion agents; and
(b) administering to said subject an effective dose of a pharmaceutical composition comprising a population of human T cells, wherein a plurality of said human T cells are chimeric antigen receptor (CAR) T cells expressing a cell surface CAR, wherein a T cell receptor (TCR) alpha gene or a TCR beta gene is inactivated in said CAR T cells, and wherein said pharmaceutical composition is administered at a dose between about 3×10⁴ to about 1×10⁷ CAR T cells/kg;
wherein said lymphodepletion regimen is administered prior to administration of said pharmaceutical composition,
wherein said CAR comprises an extracellular ligand-binding domain having specificity for a cancer cell antigen, a hinge domain, a transmembrane domain, a co-stimulatory domain, and an intracellular signaling domain,
and wherein said population of human T cells comprises one or more of the following characteristics:
(i) said CAR T cells represent between about 40% and 75% of cells in said population of human T cells;
(ii) the ratio of CD4+ CAR T cells to CD8+ CAR T cells in said population is between about 0.5 and about 3.0;
(iii) the percentage of CD4+ CAR T cells in said population that are also CCR7+ is between about 35% to about 75%; and
(iv) the percentage of CD8+ CAR T cells in said population that are also CCR7+ is between about 25% and about 50%.

2. The method of claim 1, wherein said method further comprises administering a second dose of said pharmaceutical composition to said subject.

3. The method of claim 1 or claim 2, wherein said human T cells are not derived from said subject.

4. The method of any one of claims 1-3, wherein said cancer is a cancer of B cell origin or multiple myeloma.

5. The method of any one of claims 1-4, wherein said cancer of B cell origin is acute lymphoblastic leukemia (ALL), chronic lymphocytic leukemia (CLL), small lymphocytic lymphoma (SLL), or non-Hodgkin lymphoma (NHL).

6. The method of any one of claims 1-5, wherein said cancer is mantle cell lymphoma (MCL) or diffuse large B cell lymphoma (DLBCL).

7. The method of any one of claims 1-6, wherein said subject is refractory to prior CAR T immunotherapy.

8. The method of any one of claims 1-7, wherein said lymphodepletion regimen includes administration of a biological lymphodepletion agent in an amount no greater than 1.0 mg/kg during the 7 day period preceding administration of said pharmaceutical composition.

9. The method of any one of claims 1-8, wherein said biological lymphodepletion agent is a monoclonal antibody, or a fragment thereof.

10. The method of any one of claims 1-9, wherein said monoclonal antibody, or fragment thereof, has specificity for a T cell antigen.

11. The method of any one of claims 1-10, wherein said monoclonal antibody, or fragment thereof, is an anti-CD52 monoclonal antibody, or fragment thereof, or an anti-CD3 antibody, or fragment thereof.

12. The method of any one of claims 1-11, wherein said monoclonal antibody is alemtuzumab or ALLO-647.

13. The method of any one of claims 1-12, wherein said one or more chemotherapeutic agents comprises cyclophosphamide and/or fludarabine.

14. The method of any one of claims 1-13, wherein said lymphodepletion regimen comprises administering cyclophosphamide at a dose of about 400 to about 1500 mg/m²/day.

15. The method of any one of claims 1-14, wherein said lymphodepletion regimen comprises administering cyclophosphamide at a dose of about 500 to about 1000 mg/m²/day.

16. The method of any one of claims 1-15, wherein said lymphodepletion regimen comprises administering cyclophosphamide at a dose of about 500 mg/m²/day.

17. The method of any one of claims 1-16, wherein said lymphodepletion regimen comprises administering fludarabine at a dose of about 10 to about 40 mg/m²/day.

18. The method of any one of claims 1-17, wherein said lymphodepletion regimen comprises administering fludarabine at a dose of about 30 mg/m²/day.

19. The method of any one of claims 1-18, wherein said lymphodepletion regimen comprises administering cyclophosphamide at a dose of about 500 mg/m²/day and fludarabine at a dose of about 30 mg/m²/day.

20. The method of any one of claims 1-19, wherein said lymphodepletion regimen is administered to said subject once daily starting 5 days and ending 3 days or 2 days prior to administration of said pharmaceutical composition.

21. The method of any one of claims 1-15 and 17-18, wherein said lymphodepletion regimen comprises administering cyclophosphamide at a dose of between about 500 and 1500 mg/m²/day and fludarabine at a dose of about 30 mg/m²/day.

22. The method of any one of claims 1-15 and 17-18, wherein said lymphodepletion regimen comprises administering cyclophosphamide at a dose of about 1000 mg/m²/day.

23. The method of claim 22, wherein said lymphodepletion regimen comprises administering cyclophosphamide at a dose of about 1000 mg/m²/day and fludarabine at a dose of about 30 mg/m²/day.

24. The method of any one of claims 21-23, wherein said lymphodepletion regimen is administered to said subject once daily for at least one day, or for multiple days, within 7 days prior to administration of said pharmaceutical composition.

25. The method of any one of claims 21-24, wherein said lymphodepletion regimen comprises administering cyclophosphamide once daily starting 5 days and ending 3 days prior to administration of said pharmaceutical composition, and administering fludarabine once daily starting 6 days and ending 3 days prior to administration of said pharmaceutical composition.

26. The method of any one of claims 1-25, wherein said pharmaceutical composition is administered at a dose of between about 1×10⁵ and about 6×10⁶ CAR T cells/kg.

27. The method of any one of claims 1-26, wherein said pharmaceutical composition is administered at a dose of between about 3×10⁵ and about 6×10⁶ CAR T cells/kg.

28. The method of any one of claims 1-27, wherein said pharmaceutical composition is administered at a dose of between about 3×10⁵ and about 3×10⁶ CAR T cells/kg.

29. The method of any one of claims 1-28, wherein a transgene encoding said CAR is inserted into the genome of said CAR T cells within said TCR alpha gene or said TCR beta gene, wherein said transgene disrupts expression of said TCR alpha gene or said TCR beta gene.

30. The method of any one of claims 1-29, wherein said transgene encoding said CAR is inserted into a TCR alpha constant region gene.

31. The method of claim 30, wherein said transgene encoding said CAR is inserted into an engineered meganuclease recognition sequence comprising SEQ ID NO: 1.

32. The method of any one of claims 1-31, wherein said CAR T cells do not have detectable cell surface expression of an endogenous alpha/beta TCR.

33. The method of any one of claims 1-32, wherein said CAR T cells do not have detectable cell surface expression of CD3.

34. The method of any one of claims 1-33, wherein said extracellular ligand-binding domain is a single-chain variable fragment (scFv).

35. The method of any one of claims 1-34, wherein said extracellular ligand-binding domain has specificity for CD19, CD20, orb cell maturation antigen (BCMA).

36. The method of any one of claims 1-35, wherein said extracellular ligand-binding domain is a single-chain variable fragment (scFv) comprising:
(a) a heavy chain variable domain (VH) of SEQ ID NO: 3 and a light chain variable domain (VL) of SEQ ID NO: 4; or
(h) a heavy chain variable domain (VH) of SEQ ID NO: 6 and a light chain variable domain (VL) of SEQ ID NO: 7.

37. The method of any one of claims 1-36, wherein said hinge domain is a CD8 alpha hinge domain.

38. The method of any one of claims 1-37, wherein said transmembrane domain is a CD8 alpha transmembrane domain.

39. The method of any one of claims 1-38, wherein said co-stimulatory domain comprises one or more TRAF-binding domains.

40. The method of any one of claims 1-39, wherein said co-stimulatory domain comprises a first domain comprising SEQ ID NO: 9 and a second domain comprising SEQ ID NO: 10 or 11.

41. The method of any one of claims 1-40, wherein said co-stimulatory domain is a novel 6 (N6) co-stimulatory domain or a 4-1 BB co-stimulatory domain.

42. The method of any one of claims 1-41, wherein said intracellular signaling domain is a CD3 zeta domain.

43. The method of any one of claims 1-42, wherein said CAR comprises an amino acid sequence having at least 80% sequence identity to SEQ ID NO: 5 and has specificity for CD19, or an amino acid sequence having at least 80% sequence identity to SEQ ID NO: 8 and has specificity for CD20.

44. The method of any one of claims 1-43, wherein said CAR comprises an amino acid sequence of SEQ ID NO: 5 or 8.

45. The method of any one of claims 1-44, wherein said CAR T cells represent between about 50% and about 70% of said human T cells in said population.

46. The method of any one of claims 1-45, wherein said CAR T cells represent between about 55% and about 70% of said human T cells in said population.

47. The method of any one of claims 1-46, wherein said CAR T cells represent between about 58% and about 69% of said human T cells in said population.

48. The method of any one of claims 1-47, wherein no more than about 0.5% of said human T cells in said population have detectable cell surface expression of CD3.

49. The method of any one of claims 1-48, wherein no more than about 0.3% of said human T cells in said population have detectable cell surface expression of CD3.

50. The method of any one of claims 1-49, wherein no more than about 0.2% of said human T cells in said population have detectable cell surface expression of CD3.

51. The method of any one of claims 1-50, wherein no human T cells in said population have detectable cell surface expression of CD3.

52. The method of any one of claims 1-51, wherein the ratio of CD4+ CAR T cells to CD8+ CAR T cells in said population is between about 0.7 and about 2.5.

53. The method of any one of claims 1-52, wherein the percentage of CD4+ CAR T cells in said population that are also CCR7+ is between about 35% to about 70%.

54. The method of any one of claims 1-53, wherein the percentage of CD4+ CAR T cells in said population that are also CCR7+ is between about 40% to about 68%.

55. The method of any one of claims 1-54, and wherein the percentage of CD8+ CAR T cells in said population that are also CCR7+ is between about 25% and about 45%.

56. The method of any one of claims 1-55, and wherein the percentage of CD8+ CAR T cells in said population that are also CCR7+ is between about 31% and about 42%.

57. The method of any one of claims 1-56, wherein said CAR T cells proliferate in vivo for at least one day following administration of said pharmaceutical composition.

58. The method of any one of claims 1-57, wherein said CAR T cells proliferate in vivo between about day 1 and about day 21 following administration of said pharmaceutical composition.

59. The method of any one of claims 1-58, wherein the number of copies of said CAR transgene per μg of DNA in peripheral blood mononuclear cells is elevated for up to 21 days after administration of said pharmaceutical composition when compared to the number of copies present prior to administration.

60. The method of any one of claims 1-59, wherein the number of copies of said CAR transgene per μg of DNA in peripheral blood mononuclear cells is elevated to between about 150 copies/μg to about 2100 copies/μg of DNA for at least 1 day following administration of said pharmaceutical composition.

61. The method of any one of claims 1-60, wherein the serum concentration of C-reactive protein, ferritin, IL-6, interferon gamma, or any combination thereof, is elevated compared to the concentration at day 0 for at least 1 day following administration of said pharmaceutical composition.

62. The method of any one of claims 1-61, wherein said subject achieves a partial response or a complete response to said method of immunotherapy.

63. The method of any one of claims 1-62, wherein said partial response or said complete response is maintained through at least 28 days after administration of said pharmaceutical composition.

64. The method of any one of claims 1-63, wherein said cancer is ALL, MCL, or DLBCL,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 55% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

65. The method of any one of claims 1-63, wherein said cancer is ALL, MCL, or DLBCL,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting 5 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain,
wherein said CAR T cells represent between about 58% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%,
and wherein the percentage of CD8+ CAR T cells in said population that are also CCR7+ is between about 31% and about 42%.

66. The method of any one of claims 1-63, wherein said cancer is ALL, MCL, or DLBCL,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv having specificity for CD19, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 55% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

67. The method of any one of claims 1-63, wherein said cancer is ALL, MCL, or DLBCL,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv comprising a VH domain of SEQ ID NO: 3 and a VL domain of SEQ ID NO: 4, a CD8 alpha hinge domain, a CD8 alpha transmembrane domain, an N6 co-stimulatory domain, and a CD3 zeta intracellular signaling domain,
wherein said CAR T cells represent between about 58% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%,
and wherein the percentage of CD8+ CAR T cells in said population that are also CCR7+ is between about 31% and about 42%.

68. The method of any one of claims 1-63, wherein said cancer is NHL, CLL, or SLL,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting 5 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 60% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

69. The method of any one of claims 1-63, wherein said cancer is NHL, CLL, or SLL,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain,
wherein said CAR T cells represent between about 64% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%,
and wherein the percentage of CD8+ CAR T cells in said population that are also CCR7+ is between about 31% and about 42%.

70. The method of any one of claims 1-63, wherein said cancer is NHL, CLL, or SLL,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv having specificity for CD20, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 60% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

71. The method of any one of claims 1-63, wherein said cancer is NHL, CLL, or SLL,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 3×10⁴ and 3×10⁶ CAR T cells/kg,
wherein said transgene encoding said CAR is inserted into a TCR alpha constant region gene,
wherein said CAR comprises an scFv comprising a VH domain of SEQ ID NO: 6 and a VL domain of SEQ ID NO: 7, a CD8 alpha hinge domain, a CD8 alpha transmembrane domain, an N6 co-stimulatory domain, and a CD3 zeta intracellular signaling domain,
wherein said CAR T cells represent between about 64% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%,
and wherein the percentage of CD8+ CAR T cells in said population that are also CCR7+ is between about 31% and about 42%.

72. The method of any one of claims 1-63, wherein said cancer is multiple myeloma,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting 5 days and ending 3 days prior to administration of said pharmaceutical composition,
wherein said pharmaceutical composition is administered at a dose of between about 6×10⁵ and 6×10⁶ CAR T cells/kg,
wherein said transgene encoding said CAR is inserted into a TCR alpha constant region gene,
wherein said CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 60% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

73. The method of any one of claims 1-63, wherein said cancer is multiple myeloma,
wherein said biological lymphodepletion agent is an anti-CD52 monoclonal antibody,
wherein said lymphodepletion regimen comprises administering cyclophosphamide at a dose of about 500 mg/m²/day and fludarabine at a dose of about 30 mg/m²/day,
wherein said lymphodepletion regimen is administered to said subject once daily starting 5 days and ending 3 days prior to administration of said pharmaceutical composition,
wherein said pharmaceutical composition is administered at a dose of between about 6×10⁵ and 6×10⁶ CAR T cells/kg,
wherein said transgene encoding said CAR is inserted into a TCR alpha constant region gene,
wherein said CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain,
wherein said CAR T cells represent between about 64% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%.
and wherein the percentage of CD8+ CAR T cells in said population that are also CCR7+ is between about 31% and about 42%.

74. The method of any one of claims 1-63, wherein said cancer is multiple myeloma,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 6×10⁵ and 6×10⁶ CAR T cells/kg,
wherein said transgene encoding said CAR is inserted into a TCR alpha constant region gene,
wherein said CAR comprises an scFv having specificity for BCMA, a CD8 alpha hinge domain, a CD8 alpha transmembrane domain, a co-stimulatory domain comprising one or more TRAF-binding domains, and a CD3 zeta intracellular signaling domain,
wherein said CAR T cells represent between about 60% and about 70% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 35% to about 70%,
and wherein the percentage of CD8+ CAR T cells in said population that are also CCR7+ is between about 25% and about 45%.

75. The method of any one of claims 1-63, wherein said cancer is multiple myeloma,
wherein said biological lymphodepletion agent is an anti-CD52 monoclonal antibody,
wherein said lymphodepletion regimen comprises administering cyclophosphamide at a dose of about 1000 mg/m²/day and fludarabine at a dose of about 30 mg/m²/day,
wherein said cyclophosphamide is administered to said subject once daily starting 5 days and ending 3 days prior to administration of said pharmaceutical composition, and said fludarabine is administered to said subject once daily starting 6 days and ending 3 days prior to administration of said pharmaceutical composition,
wherein said pharmaceutical composition is administered at a dose of between about 6×10⁵ and 6×10⁶ CAR T cells/kg,
wherein said transgene encoding said CAR is inserted into a TCR alpha constant region gene,
wherein said CAR comprises an scFv comprising a VH domain and a VL domain of a BCMA-specific monoclonal antibody, a CD8 alpha hinge domain, a CD8 alpha transmembrane domain, an N6 co-stimulatory domain, and a CD3 zeta intracellular signaling domain,
wherein said CAR T cells represent between about 64% and about 69% of said human T cells in said population,
wherein no more than about 0.3% of said human T cells in said population have detectable cell surface expression of CD3,
wherein the ratio of CD4+ CAR T cells to CD8+ CAR T cells in said population is between about 0.7 and about 2.5,
wherein the percentage of CD4+ CAR T cells in said population that are also CCR7+ is between about 40% to about 68%,
and wherein the percentage of CD8+ CAR T cells in said population that are also CCR7+ is between about 31% and about 42%.

76. The method of any one of claims 1-75, wherein said method further comprises manufacturing said population of human T cells, wherein said manufacturing comprises:
(a) a first culturing step wherein isolated human T cells are cultured in media for 3 days with anti-CD3 and anti-CD28 antibodies bound to a matrix or particle;
(b) electroporating said isolated human T cells to introduce mRNA encoding an engineered nuclease having specificity for a recognition sequence within said TCR alpha gene, wherein said engineered nuclease is expressed in said human T cells and generates a cleavage site at said recognition sequence:
(c) transducing said isolated human T cells with a recombinant AAV vector comprising a donor template, wherein said donor template comprises a transgene encoding said CAR, and wherein said donor template is flanked by a 5′ homology arm having homology to sequences 5′ upstream of said cleavage site, and by a 3′ homology arm having homology to sequences 3′ downstream of said cleavage site, wherein said donor template is inserted into the genome of said isolated human T cells at said cleavage site;
(d) a second culturing step wherein said isolated human T cells are cultured in media for about 5 days;
(e) removing said isolated human T cells that express cell surface CD3 using anti-CD3 antibodies; and
(f) a third culturing step wherein said isolated human T cells are cultured in media to generate said population of human T cells.

77. The method of claim 76, wherein said manufacturing is completed in about 10 days or less.

78. The method of claim 76 or claim 77, wherein said engineered nuclease is an engineered meganuclease, a zinc finger nuclease, a TALEN, a compact TALEN, a CRISPR system nuclease, or a megaTAL.

79. The method of any one of claims 76-78, wherein said engineered nuclease is an engineered meganuclease.

80. The method of claim 79, wherein said engineered meganuclease has specificity for a recognition sequence comprising SEQ ID NO: 1.

81. The method of any one of claims 78-80, wherein said engineered meganuclease comprises an amino acid sequence of SEQ ID NO: 19.


SEQUENCE LISTING (23 sequences)

SEQ ID NO: 1 - DNA, 22 bp, Homo sapiens
tggcctggagcaacaaatctga

SEQ ID NO: 2 - DNA, 22 bp, Homo sapiens
accggacctcgttgtttagact

SEQ ID NO: 3 - protein, 120 aa, Artificial Sequence
  Synthesized
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 107 aa, Artificial Sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 486 aa, Artificial Sequence
  Synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 122 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 7 - protein, 112 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 8 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 4 aa, Artificial Sequence
  Synthesized
QEED

SEQ ID NO: 10 - protein, 4 aa, Artificial Sequence
  Synthesized
EEEE

SEQ ID NO: 11 - protein, 4 aa, Artificial Sequence
  Synthesized
SEEE

SEQ ID NO: 12 - protein, 42 aa, Artificial Sequence
  Synthesized
KASRKKAAAAAKSPFASPASSAQEEDASSCRAPSEEEGSCEL

SEQ ID NO: 13 - protein, 42 aa, Artificial Sequence
  Synthesized
KRGRKKLLYIFKQPFMRPVQTTQEEDGCSCRFPEEEEGGCEL

SEQ ID NO: 14 - protein, 24 aa, Artificial Sequence
  Synthesized
TTTPAPRPPTPAPTIASQPLSLRP

SEQ ID NO: 15 - protein, 45 aa, Artificial Sequence
  Synthesized
EACRPAAGGAVHTRGLDFACDIYIWAPLAGTCGVLLLSLVITLYC

SEQ ID NO: 16 - protein, 112 aa, Artificial Sequence
  Synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 21 aa, Artificial Sequence
  Synthesized
MALPVTALLLPLALLLHAARP

SEQ ID NO: 18 - DNA, 164 bp, Artificial Sequence
  Synthesized
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 19 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 20 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 21 - protein, 9 aa, Chlamydomonas reinhardtii
LAGLIDADG

SEQ ID NO: 22 - DNA, 4627 bp, Homo sapiens
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 23 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS